(19) United States
(12) Patent Application Publication
(10) Pub. No.: US 2025/0143272 A1
(43) Pub. Date: May 8, 2025

(54) GENETICALLY MODIFIED NON-HUMAN ANIMAL WITH HUMAN OR CHIMERIC NKP46

(71) Applicant: Biocytogen Pharmaceuticals (Beijing) Co., Ltd., Beijing (CN)

(72) Inventors: Yongshun Li, Beijing (CN); Xiaofei Zhou, Beijing (CN); Jiawei Yao, Beijing (CN)

(21) Appl. No.: 18/727,810

(22) PCT Filed: Jan. 19, 2023
(86) PCT No.: PCT/CN2023/073036

§ 371 Date: Jul. 10, 2024

(30) Foreign Application Priority Data
Jan. 25, 2022 (CN) 202210084867.4

(51) Int. Cl. A01K 67/0278; C07K 14/705; C12N 15/11; C12N 15/85; G01N 33/50; G01N 33/72

(52) U.S. Cl. CPC A01K 67/0278; C07K 14/70596; C12N 15/11; C12N 15/8509; G01N 33/5011; G01N 33/5014; G01N 33/721; A01K 2207/12; A01K 2207/15; A01K 2227/105; A01K 2267/03; C12N 2015/8527; C12N 2510/00

(57) ABSTRACT

Genetically modified non-human animals that express a human or chimeric (e.g., humanized) NKP46, and methods of use thereof.

### CLAIM OF PRIORITY

This application claims the benefit of Chinese Patent Application No. 202210084867.4, filed on Jan. 25, 2022. The entire contents of the foregoing application are incorporated herein by reference.

### TECHNICAL FIELD

This disclosure relates to genetically modified animal expressing human or chimeric (e.g., humanized) NKP46, and methods of use thereof.

### BACKGROUND

The traditional drug research and development typically use in vitro screening approaches. However, these screening approaches cannot provide the body environment (such as tumor microenvironment, stromal cells, extracellular matrix components and immune cell interaction, etc.), resulting in a higher rate of failure in drug development. In addition, in view of the differences between humans and animals, the test results obtained from the use of conventional experimental animals for in vivo pharmacological test may not reflect the real disease state and the interaction at the targeting sites, resulting in that the results in many clinical trials are significantly different from the animal experimental results.

Therefore, the development of humanized animal models that are suitable for human antibody screening and evaluation will significantly improve the efficiency of new drug development and reduce the cost for drug research and development.

### SUMMARY

This disclosure is related to an animal model with human NKP46 or chimeric NKP46. The animal model can express human NKP46 or chimeric NKP46 (e.g., humanized NKP46) protein in its body. It can be used in the studies on the function of NKP46 gene, and can be used in the screening and evaluation of anti-human NKP46 antibodies or drugs targeting NKP46. In addition, the animal models prepared by the methods described herein can be used in drug screening, pharmacodynamics studies, treatments for immune-related diseases, and cancer therapy for human NKP46 target sites; they can also be used to facilitate the development and design of new drugs, and save time and cost. In summary, this disclosure provides a powerful tool for studying the function of NKP46 protein and a platform for screening cancer drugs.

In one aspect, the disclosure is related to a genetically-modified, non-human animal whose genome comprises at least one chromosome comprising a sequence encoding a human or chimeric natural cytotoxicity triggering receptor 1 (NKP46). In some embodiments, the sequence encoding the human or chimeric NKP46 is operably linked to an endogenous regulatory element at the endogenous NKP46 gene locus in the at least one chromosome. In some embodiments, the sequence encoding a human or chimeric NKP46 comprises a sequence encoding an amino acid sequence that is at least 70%, 75%, 80%, 85%, 90%, 95%, 99%, or 100% identical to human NKP46 (NP_004820.2 (SEQ ID NO: 2)). In some embodiments, the sequence encoding a human or chimeric NKP46 comprises a sequence encoding an amino acid sequence that is at least 70%, 75%, 80%, 85%, 90%, 95%, 99%, or 100% identical to SEQ ID NO: 9. In some embodiments, the sequence encoding a human or chimeric NKP46 comprises a sequence encoding an amino acid sequence that is at least 70%, 75%, 80%, 85%, 90%, 95%, 99%, or 100% identical to amino acids 1-253 or 2-253 of SEQ ID NO: 2. In some embodiments, the animal is a mammal, e.g., a monkey, a rodent, a mouse, or a rat. In some embodiments, the animal is a mouse. In some embodiments, the animal does not express endogenous NKP46 or expresses a decreased level of endogenous NKP46 as compared to NKP46 expression level in a wild-type animal. In some embodiments, the animal has one or more cells expressing human or chimeric NKP46. In some embodiments, the animal has one or more NK cells expressing human or chimeric NKP46, and the expressed human or chimeric NKP46 can induce the anti-tumor activity of the NK cells and/or bind to viral hemagglutinin.

In one aspect, the disclosure is related to a genetically-modified, non-human animal, in some embodiments, the genome of the animal comprises a replacement of a sequence encoding a region of endogenous NKP46 with a sequence encoding a corresponding region of human NKP46 at an endogenous NKP46 gene locus. In some embodiments, the sequence encoding the corresponding region of human NKP46 is operably linked to an endogenous regulatory element at the endogenous NKP46 locus, and one or more cells of the animal expresses a human or chimeric NKP46. In some embodiments, the animal does not express endogenous NKP46 or expresses a decreased level of endogenous NKP46 as compared to NKP46 expression level in a wild-type animal. In some embodiments, the replaced sequence encodes all or a portion of the extracellular region of NKP46, optionally including the signal peptide. In some embodiments, the animal has one or more cells expressing a chimeric NKP46 having a signal peptide, an extracellular region, a transmembrane region, and a cytoplasmic region, in some embodiments, the extracellular region comprises a sequence that is at least 50%, 60%, 70%, 80%, 90%, 95%, or 99% identical to the extracellular region of human NKP46 (NP 004820.2 (SEQ ID NO: 2)). In some embodiments, the extracellular region of the chimeric NKP46 has a sequence that has at least 10, 20, 30, 40, 50, 60, 70, 80, 90, 100, 110, 120, 130, 140, 150, 160, 170, 180, 190, 200, 210, 220, 230, 231, 232, 233, 234, 235, 236, or 237 contiguous amino acids that are identical to a contiguous sequence present in the extracellular region of human NKP46 (e.g., amino acids 22-253 or 22-258 of SEQ ID NO: 2). In some embodiments, the signal peptide of the chimeric NKP46 has a sequence that is at least 1, 2, 3, 4, 5, 6, 7, 8, 9, 10, 11, 12, 13, 14, 15, 16, 17, 18, 19, 20, or 21 contiguous amino acids that are identical to a contiguous sequence present in the signal peptide of human NKP46 (e.g., amino acids 1-21 or 2-21 of SEQ ID NO: 2). In some embodiments, the sequence encoding a region of endogenous NKP46 comprises exon 1, exon 2, exon 3, exon 4, exon 5, exon 6, and/or exon 7, or a part thereof, of the endogenous NKP46 gene. In some embodiments, the animal is a mouse. In some embodiments, the animal is heterozygous with respect to the replacement at the endogenous NKP46 gene locus. In some embodiments, the animal is homozygous with respect to the replacement at the endogenous NKP46 gene locus.

In one aspect, the disclosure is related to a method for making a genetically-modified, non-human animal, comprising: replacing in at least one cell of the animal, at an endogenous NKP46 gene locus, a sequence encoding a region of endogenous NKP46 with a sequence encoding a corresponding region of human NKP46. In some embodiments, the sequence encoding the corresponding region of human NKP46 comprises exon 1, exon 2, exon 3, exon 4, exon 5, exon 6, and/or exon 7, or a part thereof, of a human NKP46 gene. In some embodiments, the sequence encoding the corresponding region of human NKP46 comprises a portion of exon 1, exon 2, exon 3, exon 4, exon 5, exon 6, and a portion of exon 7, of a human NKP46 gene. In some embodiments, the sequence encoding the corresponding region of human NKP46 encodes amino acids 1-253 or 2-253 of SEQ ID NO: 2. In some embodiments, the region comprises all or a portion of the extracellular region, optionally the signal peptide, of NKP46. In some embodiments, the sequence encoding a region of endogenous NKP46 comprises exon 1, exon 2, exon 3, exon 4, exon 5, exon 6, and/or exon 7, or a part thereof, of the endogenous NKP46 gene. In some embodiments, the animal is a mouse, and the sequence encoding a region of endogenous NKP46 comprises a portion of exon 1, exon 2, exon 3, exon 4, exon 5, exon 6, and a portion of exon 7 of the endogenous NKP46 gene.

In one aspect, the disclosure is related to a non-human animal comprising at least one cell comprising a nucleotide sequence encoding a humanized NKP46 polypeptide, in some embodiments, the humanized NKP46 polypeptide comprises at least 50 contiguous amino acid residues that are identical to the corresponding contiguous amino acid sequence of a human NKP46, in some embodiments, the animal expresses the humanized NKP46 polypeptide. In some embodiments, the humanized NKP46 polypeptide has at least 50, 60, 70, 80, 90, 100, 110, 120, 130, 140, 150, 160, 170, 180, 190, 200, 210, 220, 230, 231, 232, 233, 234, 235, 236, or 237 contiguous amino acid residues that are identical to the corresponding contiguous amino acid sequence of human NKP46 extracellular region (e.g., amino acids 22-253 or 22-258 of SEQ ID NO: 2). In some embodiments, the humanized NKP46 polypeptide has at least 1, 2, 3, 4, 5, 6, 7, 8, 9, 10, 11, 12, 13, 14, 15, 16, 17, 18, 19, 20, or 21 contiguous amino acid residues that are identical to the corresponding contiguous amino acid sequence of human NKP46 signal peptide (e.g., amino acids 1-21 or 2-21 of SEQ ID NO: 2). In some embodiments, the humanized NKP46 polypeptide comprises a sequence that is at least 90%, 95%, or 99% identical to amino acids 1-253 or 2-253 of SEQ ID NO: 2. In some embodiments, the nucleotide sequence is operably linked to an endogenous NKP46 regulatory element of the animal. In some embodiments, the chimeric NKP46 polypeptide comprises an endogenous NKP46 transmembrane region and/or an endogenous NKP46 cytoplasmic region. In some embodiments, the nucleotide sequence is integrated to an endogenous NKP46 gene locus of the animal. In some embodiments, the humanized NKP46 polypeptide has at least one mouse NKP46 activity and/or at least one human NKP46 activity.

In one aspect, the disclosure is related to a method of making a genetically-modified animal cell that expresses a chimeric NKP46, the method comprising: replacing at an endogenous NKP46 gene locus, a nucleotide sequence encoding a region of endogenous NKP46 with a nucleotide sequence encoding a corresponding region of human NKP46, thereby generating a genetically-modified animal cell that includes a nucleotide sequence that encodes the chimeric NKP46, in some embodiments, the animal cell expresses the chimeric NKP46. In some embodiments, the animal is a mouse. In some embodiments, the chimeric NKP46 comprises a human or humanized NKP46 extracellular region; and a transmembrane and/or a cytoplasmic region of mouse NKP46. In some embodiments, the chimeric NKP46 further comprises a human or humanized NKP46 signal peptide. In some embodiments, the nucleotide sequence encoding the chimeric NKP46 is operably linked to an endogenous NKP46 regulatory region, e.g., promoter.

In some embodiments, the animal further comprises a sequence encoding an additional human or chimeric protein. In some embodiments, the additional human or chimeric protein is natural killer group 2D (NKG2D), epidermal growth factor receptor (EGFR), receptor tyrosine-protein kinase erbB-2 (HER2), cluster of differentiation 276 (B7H3), B-cell maturation antigen (BCMA), fibroblast activation protein alpha (FAP), C—X—C chemokine receptor type 4 (CXCR4), colony stimulating factor 2 (CSF2), tumor necrosis factor receptor 2 (TNFR2), interleukin 2 (IL-2), interleukin 15 (IL-15), interleukin 15 receptor, alpha subunit (IL-15RA), interleukin 10 (IL-10), programmed cell death protein 1 (PD-1), programmed death-ligand 1 (PD-L1), T cell immunoreceptor with Ig and ITIM domains (TIGIT), CD16A, CD2 and/or CD38.

In one aspect, the disclosure is related to a method of determining effectiveness of a therapeutic agent for the treatment of cancer, comprising: a) administering the therapeutic agent to the animal as described herein, in some embodiments, the animal has a tumor; and b) determining inhibitory effects of the therapeutic agent to the tumor. In some embodiments, the therapeutic agent is an anti-NKP46 antibody (e.g., an anti-human NKP46 antibody). In some embodiments, the tumor comprises one or more cancer cells that are injected into the animal. In some embodiments, determining inhibitory effects of the anti-NKP46 antibody to the tumor involves measuring the tumor volume in the animal. In some embodiments, the cancer is lung cancer, leukemia, colon cancer, head and neck cancer, kidney cancer, pancreatic cancer, or gastric cancer.

In one aspect, the disclosure is related to a method of determining effectiveness of an anti-NKP46 antibody and an additional therapeutic agent for the treatment of cancer, comprising a) administering the anti-NKP46 antibody and the additional therapeutic agent to the animal as described herein, in some embodiments, the animal has a tumor; and b) determining inhibitory effects on the tumor. In some embodiments, the animal further comprises a sequence encoding a human or chimeric PD-1, a human or chimeric PD-L1, and/or a human or chimeric CTLA4. In some embodiments, the additional therapeutic agent is an anti-PD-1 antibody, an anti-PD-L1 antibody, or an anti-CTLA4 antibody. In some embodiments, the tumor comprises one or more tumor cells that express PD-L1. In some embodiments, the tumor comprises one or more cancer cells that are injected into the animal. In some embodiments, determining inhibitory effects of the treatment involves measuring the tumor volume in the animal. In some embodiments, the animal has lung cancer, leukemia, colon cancer, head and neck cancer, kidney cancer, pancreatic cancer, or gastric cancer.

In one aspect, the disclosure is related to a method of determining effectiveness of a therapeutic agent for treatment an immune disorder (e.g., an autoimmune disease), comprising: a) administering the therapeutic agent to the animal as described herein, in some embodiments, the animal has the immune disorder; and b) determining effects of the therapeutic agent to the immune disorder.

In one aspect, the disclosure is related to a method of determining effectiveness of a therapeutic agent for reducing an inflammation, comprising: a) administering the therapeutic agent to the animal as described herein, in some embodiments, the animal has the inflammation; and b) determining effects of the therapeutic agent to the inflammation. In some embodiments, the inflammation is caused by a virus (e.g., influenza).

In one aspect, the disclosure is related to a method of determining toxicity of a therapeutic agent comprising: a) administering the therapeutic agent to the animal as described herein; and b) determining effects of the therapeutic agent to the animal. In some embodiments, the therapeutic agent is an anti-NKP46 antibody. In some embodiments, determining effects of the therapeutic agent to the animal involves measuring the body weight, red blood cell count, hematocrit, and/or hemoglobin of the animal.

In one aspect, the disclosure is related to a protein comprising an amino acid sequence, in some embodiments, the amino acid sequence is one of the following: (a) an amino acid sequence set forth in SEQ ID NO: 1, 2, or 9; (b) an amino acid sequence that is at least 90% identical to SEQ ID NO: 1, 2, or 9; (c) an amino acid sequence that is at least 91%, 92%, 93%, 94%, 95%, 96%, 97%, 98%, or 99% identical to SEQ ID NO: 1, 2, or 9; (d) an amino acid sequence that is different from the amino acid sequence set forth in SEQ ID NO: 1, 2, or 9 by no more than 10, 9, 8, 7, 6, 5, 4, 3, 2 or 1 amino acid; and (e) an amino acid sequence that comprises a substitution, a deletion and/or insertion of one, two, three, four, five or more amino acids to the amino acid sequence set forth in SEQ ID NO: 1, 2, or 9.

In one aspect, the disclosure is related to a nucleic acid comprising a nucleotide sequence, in some embodiments, the nucleotide sequence is one of the following: (a) a sequence that encodes the protein as described herein; (b) SEQ ID NO: 3, 4, 5, 6, 7, 8, 10, or 11; (c) a sequence that is at least 90% identical to SEQ ID NO: 3, 4, 5, 6, 7, 8, 10, or 11; and (d) a sequence that is at least 91%, 92%, 93%, 94%, 95%, 96%, 97%, 98%, or 99% identical to SEQ ID NO: 3, 4, 5, 6, 7, 8, 10, or 11.

In one aspect, the disclosure is related to a cell comprising the protein and/or the nucleic acid as described herein. In one aspect, the disclosure is related to an animal comprising the protein and/or the nucleic acid as described herein.

The disclosure further relates to a NKP46 genomic DNA sequence of a humanized mouse, a DNA sequence obtained by a reverse transcription of the mRNA obtained by transcription thereof is consistent with or complementary to the DNA sequence; a construct expressing the amino acid sequence thereof; a cell comprising the construct thereof; a tissue comprising the cell thereof.

The disclosure further relates to the use of the non-human mammal or an offspring thereof, or the tumor bearing non-human mammal, the animal model generated through the method as described herein in the development of a product related to an immunization processes of human cells, the manufacture of a human antibody, or the model system for a research in pharmacology, immunology, microbiology and medicine.

The disclosure also relates to the use of the non-human mammal or an offspring thereof, or the tumor bearing non-human mammal, the animal model generated through the method as described herein in the production and utilization of an animal experimental disease model of an immunization processes involving human cells, the study on a pathogen, or the development of a new diagnostic strategy and/or a therapeutic strategy.

The disclosure further relates to the use of the non-human mammal or an offspring thereof, or the tumor bearing non-human mammal, the animal model generated through the methods as described herein, in the screening, verifying, evaluating or studying the NKP46 gene function, human NKP46 antibodies, the drugs or efficacies for human NKP46 targeting sites, and the drugs for immune-related diseases and antitumor drugs.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a schematic diagram showing mouse and human NKP46 gene loci.

FIG. 2 is a schematic diagram showing humanized NKP46 gene locus.

FIG. 3 is a schematic diagram showing a NKP46 gene targeting strategy.

FIG. 4 is a schematic diagram showing the FRT recombination process in NKP46 gene humanized mice.

FIG. 5 is a schematic diagram showing a NKP46 gene targeting strategy.

FIGS. 6A-6B show mouse tail PCR identification results of F1 generation mice by primers L-GT-F and L-GT-R. M is a marker. WT is a wild-type control. H₂O is a water control.

FIGS. 7A-7B show Southern Blot results of cells after recombination using the A Probe and 3′ Probe. WT is a wild-type control.

FIGS. 8A-8B show the percentages of leukocyte subtypes (A) and T cell subtypes (B), respectively, in the spleen of C57BL/6 wild-type mice (+/+) and NKP46 gene humanized homozygous mice (H/H).

FIGS. 9A-9B show the percentages of leukocyte subtypes (A) and T cell subtypes (B), respectively, in the peripheral blood of C57BL/6 wild-type mice (+/+) and NKP46 gene humanized homozygous mice (H/H).

FIGS. 10A-10B show the percentages of leukocyte subtypes (A) and T cell subtypes (B), respectively, in the lymph nodes of C57BL/6 wild-type mice (+/+) and NKP46 gene humanized homozygous mice (H/H).

FIG. 11 shows the average body weight of NKP46 gene humanized homozygous mice that were xenografted with MC38 cells, and then treated with anti-human NKP46 antibody Ab1 (G2), anti-human NKP46 antibody Ab2 (G3), Ab2 with LALA mutations (G4). PBS was administered as a control (G1).

FIG. 12 shows the tumor volume of NKP46 gene humanized homozygous mice that were xenografted with MC38 cells, and then treated with anti-human NKP46 antibody Ab1 (G2), anti-human NKP46 antibody Ab2 (G3), Ab2 with LALA mutations (G4). PBS was administered as a control (G1).

FIG. 13 shows the body weight change of NKP46 gene humanized homozygous mice that were xenografted with MC38 cells, and then treated with anti-human NKP46 antibody Ab1 (G2), anti-human NKP46 antibody Ab2 (G3), Ab2 with LALA mutations (G4). PBS was administered as a control (G1).

FIG. 14 shows the alignment between human NKP46 amino acid sequence (NP_004820.2; SEQ ID NO: 2) and mouse NKP46 amino acid sequence (NP_034876.2; SEQ ID NO: 1).

FIG. 15 shows the alignment between human NKP46 amino acid sequence (NP_004820.2; SEQ ID NO: 2) and rat NKP46 amino acid sequence (NP_476547.1; SEQ ID NO: 29).

### DETAILED DESCRIPTION

This disclosure relates to transgenic non-human animal with human or chimeric (e.g., humanized) NKP46, and methods of use thereof.

Natural Killer (NK) cells constitute a population of large, granular lymphocytes that are located in the blood, lymphoid organs, such as the thymus and spleen, and non-lymphoid organs, such as the liver and uterus, as well as tissues, such as skin. NK cells were originally identified based on their ability to lyse certain tumor and virally infected cells. In contrast, normal healthy cells that express sufficient levels of MHC class I molecules are spared from NK cell attack. Subsequently, the process whereby the effector functions of developing NK cells are adapted to the levels of MHC class I expressed by a host, termed NK cell “education”, was described. It was discovered that the functional activity of NK cells was shown to be exquisitely controlled by inhibitory receptors specific for MHC class I molecules. Fully educated NK cells efficiently lyse target cells lacking MHC class I molecules, implying the existence of a set of activating NK cell receptors for non-MHC class I ligands expressed on target cells that are either not present or expressed at much lower levels on healthy cells.

In contrast to T and B lymphocytes, NK cells lack the expression of rearranging cell-surface antigen receptors and so it remained unclear how NK cells might be aroused by surface ligands expressed by tumor or virus-infected cells. Even though NK cells lack expression of the T cell receptor (TCR), they still retain expression of the & chain from the CD3 signaling complex. These data suggested that NK cells express cell-surface receptors that might share similar downstream signaling mechanisms to the TCR but are regulated by different ligands. For example, whereas T cells recognize short antigenic peptides in the context of MHC class I, NK cells can be inhibited by the expression of MHC class I molecules. Thus, NK cells express activating receptors that may recognize ligands expressed by tumor and virus-infected cells in a non-MHC-restricted fashion.

The natural cytotoxicity receptors (NCRs) were originally identified in a series of elegant redirected lysis experiments using human NK cells. The NCR family are comprised of three type I transmembrane (TM) receptors, termed NKP46, NKP44, and NKP30, which are encoded by the genes, NCR1, NCR2, and NCR3, respectively. Even though the NCRs were discovered based on their ability to induce NK cell cytotoxicity of monoclonal antibody (mAb)-coated tumor cell targets, the blocking of individual NCR activity using soluble mAbs had only a mild effect on NK cell cytotoxicity and different tumor cells varied in their susceptibility. Combinations of soluble mAbs to the NCRs were found to have a much stronger blocking effect for selected tumor cell-lines, indicating that the NCRs can cooperate with each other to mediate NK cell cytotoxicity of certain tumor cell-types. These results suggest that the coordinated expression of NCR ligands by different tumor cell-types as well as the level of NCR expression by different NK cell clones governs NK cell cytotoxicity, which is counterbalanced by the level of MHC class I expressed for a given tumor cell type.

Experimental animal models are an indispensable research tool for studying the effects of NCR-specific antibodies (e.g., anti-NKP46 (NCR1) antibodies). Common experimental animals include mice, rats, guinea pigs, hamsters, rabbits, dogs, monkeys, pigs, fish and so on. However, there are many differences between human and animal genes and protein sequences, and many human proteins cannot bind to the animal's homologous proteins to produce biological activity, leading to that the results of many clinical trials do not match the results obtained from animal experiments. A large number of clinical studies are in urgent need of better animal models. With the continuous development and maturation of genetic engineering technologies, the use of human cells or genes to replace or substitute an animal's endogenous similar cells or genes to establish a biological system or disease model closer to human, and establish the humanized experimental animal models (humanized animal model) has provided an important tool for new clinical approaches or means. In this context, the genetically engineered animal model, that is, the use of genetic manipulation techniques, the use of human normal or mutant genes to replace animal homologous genes, can be used to establish the genetically modified animal models that are closer to human gene systems. The humanized animal models have various important applications. For example, due to the presence of human or humanized genes, the animals can express or express in part of the proteins with human functions, so as to greatly reduce the differences in clinical trials between humans and animals, and provide the possibility of drug screening at animal levels.

### NKP46 (NCR1)

Natural cytotoxicity triggering receptor 1 (NCR1), also known as NKP46, is a 46 kDa type I transmembrane protein belonging to the immunoglobulin (Ig) superfamily characterized by two extracellular C2-type Ig-like domains followed by a stalk region. The two Ig-like domains of NKP46 are arranged in a V-shaped conformation positioned at an angle of 85° to each other similarly to the D1D2 domains of KIRs and Leukocyte Ig-like receptors (LILR, also known as ILTs) that share a distant ancestral evolutionary relationship with NKP46. The cytoplasmic domain of NKP46 lacks an Immunoreceptor Tyrosine-based Activation Motif (ITAM), instead the TM domain contains a positively charged arginine residue that mediates association with the negatively charged aspartate residue in the TM domain of the ITAM signaling adaptors, CD35 or the Fc receptor common γ (FcRγ).

The expression of NKP46 on NK cells is conserved across all mammalian species. Cross-linking with anti-NKP46 monoclonal antibodies (mAbs) results in calcium release and the secretion of IFN-γ and TNF-α by NK cells and blocking NKP46 signaling with specific mAbs can result in reduced NK cell cytotoxicity of certain tumor cell-lines, although the most potent blocking activity is observed in combination with mAbs to other NCRs. Studies have shown that NKP46 is also expressed by innate lymphoid cells (ILCs) of group 1 (ILC1) and a subset of group 3 ILCs (NCR⁺ ILC3), γδ T cells, a population of oligoclonally expanded intraepithelial (IEL) cytotoxic T lymphocytes (CTL) and a population of IL-15-dependent innate-like IEL lacking surface TCR expression in celiac disease patients, and umbilical cord blood (UCB) T cells cultured in IL-15. NKP46 is also expressed by malignant NK, NKT, and T cell lymphomas.

A detailed description of NKP46 and its function can be found, e.g., in Barrow, A. D., et al. “The natural cytotoxicity receptors in health and disease.” Frontiers in Immunology 10 (2019): 909; Liu, S., et al. “NK cell-based cancer immunotherapy: From basic biology to clinical development.” Journal of Hematology & Oncology 14.1 (2021): 1-17; Zamai, L., et al. “Understanding the synergy of NKp46 and co-activating signals in various NK cell subpopulations: paving the way for more successful NK-cell-based immunotherapy.” Cells 9.3 (2020): 753; and Hadad, U., et al. “NKp46 clusters at the immune synapse and regulates NK cell polarization.” Frontiers in Immunology 6 (2015): 495; each of which is incorporated by reference in its entirety.

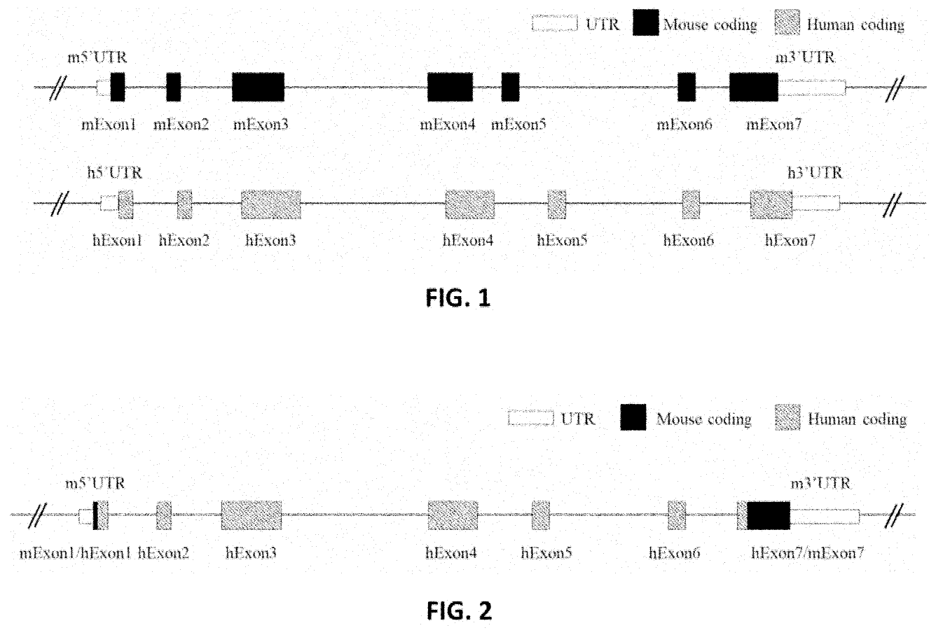

In human genomes, NKP46 gene (Gene ID: 9437) locus has seven exons, exon 1, exon 2, exon 3, exon 4, exon 5, exon 6, and exon 7 (FIG. 1). The human NKP46 protein also has, from N-terminus to C-terminus, a signal peptide, an extracellular region, a transmembrane region, and a cytoplasmic region. The nucleotide sequence for human NKP46 mRNA is NM_004829.7, and the amino acid sequence for human NKP46 is NP_004820.2 (SEQ ID NO: 2). The location for each exon and each region in human NKP46 nucleotide sequence and amino acid sequence is listed below:

TABLE 1

| | | NP_004820.2 |
|---|---|---|
| Human NKP46 (NCR1) | NM_004829.7 | 304 aa |
| (approximate location) | 1157 bp | SEQ ID NO: 2 |
| Exon 1 | 1-74 | 1-11 |
| Exon 2 | 75-110 | 12-23 |
| Exon 3 | 111-395 | 24-118 |
| Exon 4 | 396-674 | 119-211 |
| Exon 5 | 675-722 | 212-227 |
| Exon 6 | 723-773 | 228-244 |
| Exon 7 | 774-1157 | 245-304 |
| Signal peptide | 41-103 | 1-21 |
| Extracellular | 104-814 | 22-258 |
| Transmembrane | 815-877 | 259-279 |
| Cytoplasmic | 878-952 | 280-304 |
| Donor region in Example | 44-799 | 2-253 |

The human NKP46 gene (Gene ID: 9437) is located in Chromosome 19 of the human genome, which is located from 54906063 to 54938211 (GRCh38.p13 (GCF_000001405.39)). The 5′-UTR is from 54906148 to 54906187, exon 1 is from 54906148 to 54906221, intron 1 is from 54906222 to 54906298, exon 2 is from 54906299 to 54906334, intron 2 is from 54906335 to 54906522, exon 3 is from 54906523 to 54906807, intron 3 is from 54906808 to 54909244, exon 4 is from 54909245 to 54909523, intron 4 is from 54909524 to 54910017, exon 5 is from 54910018 to 54910065, intron 5 is from 54910066 to 54912167, exon 6 is from 54912168 to 54912218, intron 6 is from 54912219 to 54912689, exon 7 is from 54912690 to 54913073, and the 3′-UTR is from 54912872 to 54913073, based on transcript NM_004829.7. All relevant information for human NKP46 locus can be found in the NCBI website with Gene ID: 9437, which is incorporated by reference herein in its entirety.

According to UniProt ID: 076036, the extracellular region of human NKP46 includes two Ig-like domains: Ig-like 1 domain and Ig-like 2 domain. The Ig-like 1 domain corresponds to amino acids 34-118 of SEQ ID NO: 2. The Ig-like 2 domain corresponds to amino acids 129-211 of SEQ ID NO: 2.

In mice, NKP46 gene locus has seven exons, exon 1, exon 2, exon 3, exon 4, exon 5, exon 6, and exon 7 (FIG. 1). The mouse NKP46 protein also has, from N-terminus to C-terminus, a signal peptide, an extracellular region, a transmembrane region, and a cytoplasmic region. The nucleotide sequence for mouse NKP46 mRNA is NM_010746.3, the amino acid sequence for mouse NKP46 is NP_034876.2 (SEQ ID NO: 1). The location for each exon and each region in the mouse NKP46 nucleotide sequence and amino acid sequence is listed below:

TABLE 2

| | | NP_034876.2 |
|---|---|---|
| Mouse NKP46 (NCR1) | NM_010746.3 | 325 aa |
| (approximate location) | 1463 bp | SEQ ID NO: 1 |
| Exon 1 | 1-59 | 1-11 |
| Exon 2 | 60-95 | 12-23 |
| Exon 3 | 96-380 | 24-118 |
| Exon 4 | 381-659 | 119-211 |
| Exon 5 | 660-710 | 212-228 |
| Exon 6 | 711-758 | 229-244 |
| Exon 7 | 759-1463 | 245-325 |
| Signal peptide | 26-73 | 1-16 |
| Extracellular | 74-790 | 17-255 |
| Transmembrane | 791-844 | 256-273 |
| Cytoplasmic | 845-1000 | 274-325 |
| Replaced region in Example | 29-784 | 2-253 |

The mouse NKP46 gene (Gene ID: 17086) is located in Chromosome 7 of the mouse genome, which is located from 4340714 to 4348183 (GRCm39 (GCF_000001635.27)). The 5′-UTR is from 4340723 to 4340747, exon 1 is from 4340723 to 4340781, intron 1 is from 4340782 to 4340858, exon 2 is from 4340859 to 4340894, intron 2 is from 4340895 to 4341080, exon 3 is from 4341081 to 4341365, intron 3 is from 4341366 to 4343758, exon 4 is from 4343759 to 4344037, intron 4 is from 4344038 to 4344247, exon 5 is from 4344248 to 4344298, intron 5 is from 4344299 to 4347136, exon 6 is from 4347137 to 4347184, intron 6 is from 4347185 to 4347458, exon 7 is from 4347459 to 4348163, and the 3′-UTR is from 4347704 to 4348163, based on transcript NM_010746.3. All relevant information for mouse Ncr1 locus can be found in the NCBI website with Gene ID: 17086, which is incorporated by reference herein in its entirety.

According to UniProt ID: Q8C567, the extracellular region of mouse NKP46 includes two Ig-like domains: Ig-like 1 domain and Ig-like 2 domain. The Ig-like 1 domain corresponds to amino acids 34-118 of SEQ ID NO: 1. The Ig-like 2 domain corresponds to amino acids 129-211 of SEQ ID NO: 1.

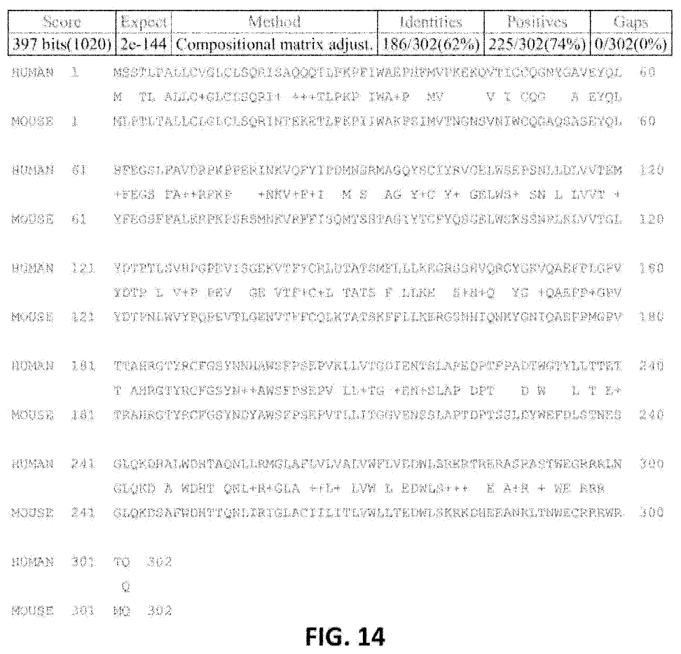

FIG. 14 shows the alignment between human NKP46 amino acid sequence (NP_004820.2; SEQ ID NO: 2) and mouse NKP46 amino acid sequence (NP_034876.2; SEQ ID NO: 1). Thus, the corresponding amino acid residue or region between human and mouse NKP46 can be found in FIG. 14.

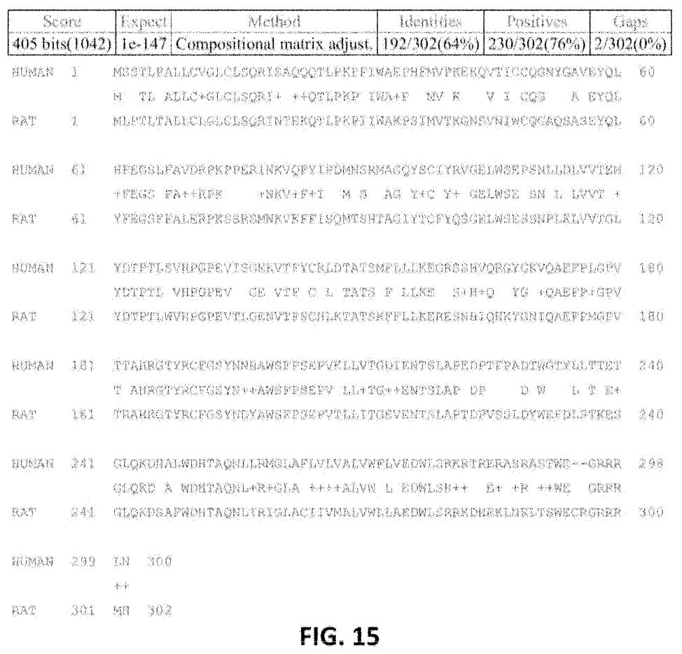

NKP46 genes, proteins, and locus of the other species are also known in the art. For example, the gene ID for NKP46 (NCR1) in Rattus norvegicus (rat) is 117547, the gene ID for NKP46 in Macaca mulatta (Rhesus monkey) is 574242, the gene ID for NKP46 in Canis lupus familiaris (dog) is 611390, and the gene ID for NKP46 in Sus scrofa (pig) is 100141419. The relevant information for these genes (e.g., intron sequences, exon sequences, amino acid residues of these proteins) can be found, e.g., in NCBI database, which is incorporated by reference herein in its entirety. FIG. 15 shows the alignment between human NKP46 amino acid sequence (NP_004820.2; SEQ ID NO: 2) and rat NKP46 amino acid sequence (NP_476547.1; SEQ ID NO: 29. Thus, the corresponding amino acid residue or region between human and rodent NKP46 can be found in FIG. 15.

The present disclosure provides human or chimeric (e.g., humanized) NKP46 nucleotide sequence and/or amino acid sequences. In some embodiments, the entire sequence of mouse exon 1, exon 2, exon 3, exon 4, exon 5, exon 6, exon 7, signal peptide, extracellular region, transmembrane region, and/or cytoplasmic region are replaced by the corresponding human sequence. In some embodiments, a “region” or “portion” of mouse exon 1, exon 2, exon 3, exon 4, exon 5, exon 6, exon 7, signal peptide, extracellular region, transmembrane region, and/or cytoplasmic region are replaced by the corresponding human sequence. The term “region” or “portion” can refer to at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 250, 300, 350, 400, 450, 500, 550, 600, 650, 700, 750, 755, or 756 nucleotides, or at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 245, 250, 251, or 252 amino acid residues. In some embodiments, the “region” or “portion” can be at least 50%, 55%, 60%, 65%, 70%, 75%, 80%, 85%, 90%, 95%, or 99% identical to exon 1, exon 2, exon 3, exon 4, exon 5, exon 6, exon 7, signal peptide, extracellular region, transmembrane region, or cytoplasmic region. In some embodiments, a region, a portion, or the entire sequence of mouse exon 1, exon 2, exon 3, exon 4, exon 5, exon 6, and/or exon 7 (e.g., a portion of exon 1, exons 2-6, and a portion of exon 7) are replaced by a region, a portion, or the entire sequence of the human exon 1, exon 2, exon 3, exon 4, exon 5, exon 6, and/or exon 7 (e.g., a portion of exon 1, exons 2-6, and a portion of exon 7).

In some embodiments, a “region” or “portion” of the signal peptide, extracellular region, transmembrane region, cytoplasmic region, exon 1, exon 2, exon 3, exon 4, exon 5, exon 6, and/or exon 7 is deleted.

In some embodiments, the present disclosure is related to a genetically-modified, non-human animal whose genome comprises a chimeric (e.g., humanized) NKP46 nucleotide sequence. In some embodiments, the chimeric (e.g., humanized) NKP46 nucleotide sequence encodes a NKP46 protein comprising a signal peptide, an extracellular region, a transmembrane region, and a cytoplasmic region. In some embodiments, the signal peptide comprises a sequence that is at least 80%, 85%, 90%, 95%, or 100% identical to amino acids 1-21 or 2-21 of SEQ ID NO: 2. In some embodiments, the signal peptide comprises all or part of human NKP46 signal peptide. In some embodiments, the extracellular region comprises a sequence that is at least 80%, 85%, 90%, 95%, or 100% identical to amino acids 22-253 or 22-258 of SEQ ID NO: 2. In some embodiments, the extracellular region comprises all or part of human NKP46 extracellular region. In some embodiments, the extracellular region comprises at least 1, 2, 3, 4, or 5 amino acids at the C-terminus of endogenous NKP46 extracellular region. In some embodiments, the transmembrane region comprises a sequence that is at least 80%, 85%, 90%, 95%, or 100% identical to amino acids 256-273 of SEQ ID NO: 1. In some embodiments, the transmembrane region comprises all or part of endogenous NKP46 transmembrane region. In some embodiments, the cytoplasmic region comprises a sequence that is at least 80%, 85%, 90%, 95%, or 100% identical to amino acids 274-325 of SEQ ID NO: 1. In some embodiments, the cytoplasmic region comprises all or part of endogenous NKP46 cytoplasmic region. In some embodiments, the genome of the animal comprises a sequence that is at least 80%, 85%, 90%, 95%, or 100% identical to SEQ ID NO: 3, 4, 5, 6, 7, 8, 10, or 11.

In some embodiments, the genetically-modified non-human animal described herein comprises a sequence encoding a human or humanized NKP46 protein. In some embodiments, the NKP46 protein comprises, from N-terminus to C-terminus, a signal peptide, an extracellular region, a transmembrane region, and a cytoplasmic region. In some embodiments, the humanized NKP46 protein comprises a human or humanized signal peptide. In some embodiments, the humanized NKP46 protein comprises an endogenous signal peptide. In some embodiments, the humanized NKP46 protein comprises a human or humanized extracellular region. In some embodiments, the humanized NKP46 protein comprises an endogenous extracellular region. In some embodiments, the humanized NKP46 protein comprises a human or humanized transmembrane region. In some embodiments, the humanized NKP46 protein comprises an endogenous transmembrane region. In some embodiments, the humanized NKP46 protein comprises a human or humanized cytoplasmic region. In some embodiments, the humanized NKP46 protein comprises an endogenous cytoplasmic region. In some embodiments, the humanized NKP46 protein comprises a human or humanized signal peptide, a human or humanized extracellular region, an endogenous transmembrane region, and an endogenous cytoplasmic region. In some embodiments, the humanized NKP46 protein comprises an endogenous sequence that corresponds to amino acids 254-325 or 256-325 of SEQ ID NO: 1.

In some embodiments, the extracellular region of the NKP46 protein comprises a Ig-like 1 domain and a Ig-like 2 domain. In some embodiments, the Ig-like 1 domain is humanized. In some embodiments, the Ig-like 2 domain is humanized. In some embodiments, both the Ig-like 1 domain and the Ig-like 2 domain are humanized.

In some embodiments, the genetically-modified non-human animal described herein comprises a human or humanized NKP46 gene. In some embodiments, the humanized NKP46 gene comprises 7 exons. In some embodiments, the humanized NKP46 gene comprises humanized exon 1, human exon 2, human exon 3, human exon 4, human exon 5, human exon 6, and/or humanized exon 7. In some embodiments, the humanized NKP46 gene comprises 6 introns. In some embodiments, the humanized NKP46 gene comprises human intron 1, human intron 2, human intron 3, human intron 4, human intron 5, and/or human intron 6. In some embodiments, the humanized NKP46 gene comprises human or humanized 5′ UTR. In some embodiments, the humanized NKP46 gene comprises human or humanized 3′ UTR. In some embodiments, the humanized NKP46 gene comprises endogenous 5′ UTR. In some embodiments, the humanized NKP46 gene comprises endogenous 3′ UTR.

Thus, in some embodiments, the present disclosure also provides a chimeric (e.g., humanized) NKP46 nucleotide sequence and/or amino acid sequences, wherein in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from mouse NKP46 mRNA sequence (e.g., NM_010746.3), mouse NKP46 amino acid sequence (e.g., SEQ ID NO: 1), or a portion thereof (e.g., 5′ UTR, a portion of exon 1, a portion of exon 7, and 3′ UTR); and in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from human NKP46 mRNA sequence (e.g., NM_004829.7), human NKP46 amino acid sequence (e.g., SEQ ID NO: 2), or a portion thereof (e.g., a portion of exon 1, exons 2-6, and a portion of exon 7).

In some embodiments, the sequence encoding amino acids 2-253 of mouse NKP46 (SEQ ID NO: 1) is replaced. In some embodiments, the sequence is replaced by a sequence encoding a corresponding region of human NKP46 (e.g., amino acids 2-253 of human NKP46 (SEQ ID NO: 2)).

In some embodiments, the sequence encoding amino acids 1-253 of mouse NKP46 (SEQ ID NO: 1) is replaced. In some embodiments, the sequence is replaced by a sequence encoding a corresponding region of human NKP46 (e.g., amino acids 1-253 of human NKP46 (SEQ ID NO: 2)).

In some embodiments, the sequence encoding amino acids 22-253 of mouse NKP46 (SEQ ID NO: 1) is replaced. In some embodiments, the sequence is replaced by a sequence encoding a corresponding region of human NKP46 (e.g., amino acids 22-253 of human NKP46 (SEQ ID NO: 2)).

In some embodiments, the sequence encoding amino acids 2-16, 17-253, or 2-253 of mouse NKP46 (SEQ ID NO: 1) is replaced. In some embodiments, the sequence is replaced by a sequence encoding a corresponding region of human NKP46 (e.g., amino acids 2-21, 22-253, or 2-253 of human NKP46 (SEQ ID NO: 2)).

In some embodiments, the nucleic acids as described herein are operably linked to a promotor or regulatory element, e.g., an endogenous mouse NKP46 promotor, an inducible promoter, an enhancer, and/or mouse or human regulatory elements.

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 150, 200, 250, 300, 350, 400, 450, 500, 550, 600, 650, 700, 750, 751, 752, 753, 754, 755, or 756 nucleotides, e.g., contiguous or non-contiguous nucleotides) that are different from part of or the entire mouse NKP46 nucleotide sequence (e.g., a portion of exon 1, exons 2-6, and a portion of exon 7 of NM_010746.3).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 150, 200, 250, 300, 350, 400, 450, 500, 550, 600, 650, 700, 705, 706, or 707 nucleotides, e.g., contiguous or non-contiguous nucleotides) that is the same as part of or the entire mouse NKP46 nucleotide sequence (e.g., 5′ UTR, a portion of exon 1, a portion of exon 7, and 3′ UTR of NM_010746.3).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 150, 200, 250, 300, 350, 380, 390, or 400 nucleotides, e.g., contiguous or non-contiguous nucleotides) that is different from part of or the entire human NKP46 nucleotide sequence (e.g., 5′ UTR, a portion of exon 1, a portion of exon 7, and 3′ UTR of NM_004829.7).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 150, 200, 250, 300, 350, 400, 450, 500, 550, 600, 650, 700, 750, 751, 752, 753, 754, 755, or 756 nucleotides, e.g., contiguous or non-contiguous nucleotides) that is the same as part of or the entire human NKP46 nucleotide sequence (e.g., a portion (at least 20 bp) of exon 1, exons 2-6, and a portion (at least 10 bp) of exon 7 of NM_004829.7).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 110, 120, 130, 140, 150, 160, 170, 180, 190, 200, 210, 220, 230, 240, 250, 251, 252, or 253 amino acid residues, e.g., contiguous or non-contiguous amino acid residues) that is different from part of or the entire mouse NKP46 amino acid sequence (e.g., amino acids 1-253 or 2-253 of NP_034876.2 (SEQ ID NO: 1)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 71, 72, or 73 amino acid residues, e.g., contiguous or non-contiguous amino acid residues) that is the same as part of or the entire mouse NKP46 amino acid sequence (e.g., amino acids 254-325 of NP_034876.2 (SEQ ID NO: 1)).

In some embodiments, the amino acid sequence has at least a portion (e.g., at least 1, 2, 3, 4, 5, 6, 7, 8, 9, 10, 11, 12, 13, 14, 15, 20, 30, 40, 50, 51, or 52 amino acid residues, e.g., contiguous or non-contiguous amino acid residues) that is different from part of or the entire human NKP46 amino acid sequence (e.g., amino acids 254-304 of NP_004820.2 (SEQ ID NO: 2)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 110, 120, 130, 140, 150, 160, 170, 180, 190, 200, 210, 220, 230, 240, 250, 251, 252, or 253 amino acid residues, e.g., contiguous or non-contiguous amino acid residues) that is the same as part of or the entire human NKP46 amino acid sequence (e.g., amino acids 1-253 or 2-253 of NP_004820.2 (SEQ ID NO: 2)).

The present disclosure also provides a humanized NKP46 mouse amino acid sequence, wherein the amino acid sequence is selected from the group consisting of:
a) an amino acid sequence shown in SEQ ID NO: 1, 2, or 9;
b) an amino acid sequence having a homology of at least 90% with or at least 90% identical to the amino acid sequence shown in SEQ ID NO: 1, 2, or 9;
c) an amino acid sequence encoded by a nucleic acid sequence, wherein the nucleic acid sequence is able to hybridize to a nucleotide sequence encoding the amino acid shown in SEQ ID NO: 1, 2, or 9 under a low stringency condition or a strict stringency condition;
d) an amino acid sequence having a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the amino acid sequence shown in SEQ ID NO: 1, 2, or 9;
e) an amino acid sequence that is different from the amino acid sequence shown in SEQ ID NO: 1, 2, or 9 by no more than 10, 9, 8, 7, 6, 5, 4, 3, 2 or no more than 1 amino acid; or
f) an amino acid sequence that comprises a substitution, a deletion and/or insertion of one or more amino acids to the amino acid sequence shown in SEQ ID NO: 1, 2, or 9.

The present disclosure also provides a humanized NKP46 amino acid sequence, wherein the amino acid sequence is selected from the group consisting of:
a) all or part of amino acids 2-253 or 22-253 of SEQ ID NO: 2;
b) an amino acid sequence have a homology of at least 90%, 91%, 92%, 93%, 94%, 95%, 96%, 97%, 98%, or 99% to amino acids 2-253 or 22-253 of SEQ ID NO: 2;
c) an amino acid sequence that is different from amino acids 2-253 or 22-253 of SEQ ID NO: 2 by no more than 10, 9, 8, 7, 6, 5, 4, 3, 2 or no more than 1 amino acid; and
d) an amino acid sequence that comprises a substitution, a deletion and/or insertion of one or more amino acids to amino acids 2-253 or 22-253 of SEQ ID NO: 2.

The present disclosure also provides a humanized NKP46 amino acid sequence, wherein the amino acid sequence is selected from the group consisting of:
a) all or part of amino acids 254-325 of SEQ ID NO: 1;
b) an amino acid sequence have a homology of at least 90%, 91%, 92%, 93%, 94%, 95%, 96%, 97%, 98%, or 99% to amino acids 254-325 of SEQ ID NO: 1;
c) an amino acid sequence that is different from amino acids 254-325 of SEQ ID NO: 1 by no more than 10, 9, 8, 7, 6, 5, 4, 3, 2 or no more than 1 amino acid; and
d) an amino acid sequence that comprises a substitution, a deletion and/or insertion of one or more amino acids to amino acids 254-325 of SEQ ID NO: 1.

The present disclosure also relates to a NKP46 nucleic acid (e.g., DNA or RNA) sequence, wherein the nucleic acid sequence can be selected from the group consisting of:
a) a nucleic acid sequence as shown in SEQ ID NO: 3, 4, 5, 6, 7, 8, 10, or 11, or a nucleic acid sequence encoding a homologous NKP46 amino acid sequence of a humanized mouse NKP46;
b) a nucleic acid sequence that is able to hybridize to the nucleotide sequence as shown in SEQ ID NO: 3, 4, 5, 6, 7, 8, 10, or 11 under a low stringency condition or a strict stringency condition;
c) a nucleic acid sequence that has a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the nucleotide sequence as shown in SEQ ID NO: 3, 4, 5, 6, 7, 8, 10, or 11;
d) a nucleic acid sequence that encodes an amino acid sequence, wherein the amino acid sequence has a homology of at least 90% with or at least 90% identical to the amino acid sequence shown in SEQ ID NO: 1, 2, or 9;
e) a nucleic acid sequence that encodes an amino acid sequence, wherein the amino acid sequence has a homology of at least 90%, 91%, 92%, 93%, 94%, 95%, 96%, 97%, 98%, or 99% with, or at least 90%, 91%, 92%, 93%, 94%, 95%, 96%, 97%, 98%, or 99% identical to the amino acid sequence shown in SEQ ID NO: 1, 2, or 9;
f) a nucleic acid sequence that encodes an amino acid sequence, wherein the amino acid sequence is different from the amino acid sequence shown in SEQ ID NO: 1, 2, or 9 by no more than 10, 9, 8, 7, 6, 5, 4, 3, 2 or no more than 1 amino acid; and/or
g) a nucleic acid sequence that encodes an amino acid sequence, wherein the amino acid sequence comprises a substitution, a deletion and/or insertion of one or more amino acids to the amino acid sequence shown in SEQ ID NO: 1, 2, or 9.

The present disclosure further relates to a NKP46 genomic DNA sequence of a humanized mouse. The DNA sequence is obtained by reverse transcription of the mRNA obtained by transcription thereof is consistent with or complementary to the DNA sequence homologous to the sequence shown in SEQ ID NO: 7 or 8.

The disclosure also provides an amino acid sequence that has a homology of at least 90% with, or at least 90% identical to the sequence shown in SEQ ID NO: 1, 2, or 9, and has protein activity. In some embodiments, the homology with the sequence shown in SEQ ID NO: 1, 2, or 9 is at least about 90%, 91%, 92%, 93%, 94%, 95%, 96%, 97%, 98%, or at least 99%. In some embodiments, the foregoing homology is at least about 60%, 61%, 62%, 63%, 64%, 65%, 66%, 67%, 68%, 69%, 70%, 71%, 72%, 73%, 74%, 75%, 80%, or 85%.

In some embodiments, the percentage identity with the sequence shown in SEQ ID NO: 1, 2, or 9 is at least about 90%, 91%, 92%, 93%, 94%, 95%, 96%, 97%, 98%, or at least 99%. In some embodiments, the foregoing percentage identity is at least about 60%, 61%, 62%, 63%, 64%, 65%, 66%, 67%, 68%, 69%, 70%, 71%, 72%, 73%, 74%, 75%, 80%, or 85%.

The disclosure also provides a nucleotide sequence that has a homology of at least 90%, or at least 90% identical to the sequence shown in SEQ ID NO: 7 or 8, and encodes a polypeptide that has protein activity. In some embodiments, the homology with the sequence shown in SEQ ID NO: 7 or 8 is at least about 90%, 91%, 92%, 93%, 94%, 95%, 96%, 97%, 98%, or at least 99%. In some embodiments, the foregoing homology is at least about 50%, 55%, 60%, 65%, 66%, 67%, 68%, 69%, 70%, 71%, 72%, 73%, 74%, 75%, 80%, or 85%.

In some embodiments, the percentage identity with the sequence shown in SEQ ID NO: 3, 4, 5, 6, 7, 8, 10, or 11 is at least about 90%, 91%, 92%, 93%, 94%, 95%, 96%, 97%, 98%, or at least 99%. In some embodiments, the foregoing percentage identity is at least about 50%, 55%, 60%, 65%, 66%, 67%, 68%, 69%, 70%, 71%, 72%, 73%, 74%, 75%, 80%, or 85%.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein. In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 450, 500, 550, 600, 650, 700, 750, 800, 850, 900, 950, 1000, 1100, 1200, 1300, or 1400 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, 200, 210, 220, 230, 240, 245, 250, 251, 252, 260, 270, 280, 290, 300, 304, 310, 320, or 325 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

The percentage of residues conserved with similar physicochemical properties (percent homology), e.g. leucine and isoleucine, can also be used to measure sequence similarity. Families of amino acid residues having similar physicochemical properties have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The homology percentage, in many cases, is higher than the identity percentage.

Cells, tissues, and animals (e.g., mouse) are also provided that comprise the nucleotide sequences as described herein, as well as cells, tissues, and animals (e.g., mouse) that express human or chimeric (e.g., humanized) NKP46 from an endogenous non-human NKP46 locus.

### Genetically Modified Animals

As used herein, the term “genetically-modified non-human animal” refers to a non-human animal having exogenous DNA in at least one chromosome of the animal's genome. In some embodiments, at least one or more cells, e.g., at least 1%, 2%, 3%, 4%, 5%, 10%, 20%, 30%, 40%, 50% of cells of the genetically-modified non-human animal have the exogenous DNA in its genome. The cell having exogenous DNA can be various kinds of cells, e.g., an endogenous cell, a somatic cell, an immune cell, a T cell, a B cell, an antigen presenting cell, a macrophage, a dendritic cell, a germ cell, a blastocyst, or an endogenous tumor cell. In some embodiments, genetically-modified non-human animals are provided that comprise a modified endogenous NKP46 locus that comprises an exogenous sequence (e.g., a human sequence), e.g., a replacement of one or more non-human sequences with one or more human sequences. The animals are generally able to pass the modification to progeny, i.e., through germline transmission.

As used herein, the term “chimeric gene” or “chimeric nucleic acid” refers to a gene or a nucleic acid, wherein two or more portions of the gene or the nucleic acid are from different species, or at least one of the sequences of the gene or the nucleic acid does not correspond to the wild-type nucleic acid in the animal. In some embodiments, the chimeric gene or chimeric nucleic acid has at least one portion of the sequence that is derived from two or more different sources, e.g., sequences encoding different proteins or sequences encoding the same (or homologous) protein of two or more different species. In some embodiments, the chimeric gene or the chimeric nucleic acid is a humanized gene or humanized nucleic acid.

As used herein, the term “chimeric protein” or “chimeric polypeptide” refers to a protein or a polypeptide, wherein two or more portions of the protein or the polypeptide are from different species, or at least one of the sequences of the protein or the polypeptide does not correspond to wild-type amino acid sequence in the animal. In some embodiments, the chimeric protein or the chimeric polypeptide has at least one portion of the sequence that is derived from two or more different sources, e.g., same (or homologous) proteins of different species. In some embodiments, the chimeric protein or the chimeric polypeptide is a humanized protein or a humanized polypeptide.

As used herein, the term “humanized protein” or “humanized polypeptide” refers to a protein or a polypeptide, wherein at least a portion of the protein or the polypeptide is from the human protein or human polypeptide. In some embodiments, the humanized protein or polypeptide is a human protein or polypeptide.

As used herein, the term “humanized nucleic acid” refers to a nucleic acid, wherein at least a portion of the nucleic acid is from the human. In some embodiments, the entire nucleic acid of the humanized nucleic acid is from human. In some embodiments, the humanized nucleic acid is a humanized exon. A humanized exon can be, e.g., a human exon or a chimeric exon.

In some embodiments, the chimeric gene or the chimeric nucleic acid is a humanized NKP46 gene or a humanized NKP46 nucleic acid. In some embodiments, at least one or more portions of the gene or the nucleic acid is from the human NKP46 gene, at least one or more portions of the gene or the nucleic acid is from a non-human NKP46 gene. In some embodiments, the gene or the nucleic acid comprises a sequence that encodes an NKP46 protein. The encoded NKP46 protein is functional or has at least one activity of the human NKP46 protein or the non-human NKP46 protein, e.g., activating NK cells.

In some embodiments, the humanized NKP46 gene includes a nucleotide sequence of 20 bp-32149 bp (contiguous or non-contiguous) that is identical to human NKP46 gene. In some embodiments, the nucleotide sequence is 20-6525 bp, or 20-756 bp, e.g., 20, 50, 100, 200, 300, 400, 500, 600, 700, 750, 756, 800, 900, 1000, 1100, 1200, 1300, 1400, 1500, 1600, 1700, 1800, 1900, 2000, 2500, 3000, 4000, 4500, 5000, 5500, 6000, 6500, 6525, 7000, 7500, 8000, 8500, 9000, 9500, 10000, 20000, or 30000 bp.

In some embodiments, the chimeric protein or the chimeric polypeptide is a humanized NKP46 protein or a humanized NKP46 polypeptide. In some embodiments, at least one or more portions of the amino acid sequence of the protein or the polypeptide is from a human NKP46 protein, and at least one or more portions of the amino acid sequence of the protein or the polypeptide is from a non-human NKP46 protein. The humanized NKP46 protein or the humanized NKP46 polypeptide is functional or has at least one activity of the human NKP46 protein or the non-human NKP46 protein.

In some embodiments, the humanized NKP46 protein includes a polypeptide sequence of 5-304 amino acids (contiguous or non-contiguous) that is identical to human NKP46 protein. In some embodiments, the polypeptide sequence is 10-253 or 10-252 amino acids in length, e.g., 5, 10, 20, 30, 40, 50, 60, 70, 80, 90, 100, 110, 120, 130, 140, 150, 160, 170, 180, 190, 200, 210, 220, 230, 240, 250, 252, 253, 260, 270, 280, 290, 300, or 304 amino acids.

In some embodiments, the extracellular region is human or humanized. In some embodiments, the signal peptide is human or humanized. In some embodiments, the cytoplasmic region is human or humanized. In some embodiments, the transmembrane region is human or humanized. In some embodiments, both the extracellular region and signal peptide are human or humanized. In some embodiments, both the transmembrane and cytoplasmic regions are endogenous.

The genetically modified non-human animal can be various animals, e.g., a mouse, rat, rabbit, pig, bovine (e.g., cow, bull, buffalo), deer, sheep, goat, chicken, cat, dog, ferret, primate (e.g., marmoset, rhesus monkey). For the non-human animals where suitable genetically modifiable embryonic stem (ES) cells are not readily available, other methods are employed to make a non-human animal comprising the genetic modification. Such methods include, e.g., modifying a non-ES cell genome (e.g., a fibroblast or an induced pluripotent cell) and employing nuclear transfer to transfer the modified genome to a suitable cell, e.g., an oocyte, and gestating the modified cell (e.g., the modified oocyte) in a non-human animal under suitable conditions to form an embryo. These methods are known in the art, and are described, e.g., in A. Nagy, et al., “Manipulating the Mouse Embryo: A Laboratory Manual (Third Edition),” Cold Spring Harbor Laboratory Press, 2003, which is incorporated by reference herein in its entirety.

In one aspect, the animal is a mammal, e.g., of the superfamily Dipodoidea or Muroidea. In some embodiments, the genetically modified animal is a rodent. The rodent can be selected from a mouse, a rat, and a hamster. In some embodiments, the genetically modified animal is from a family selected from Calomyscidae (e.g., mouse-like hamsters), Cricetidae (e.g., hamster, New World rats and mice, voles), Muridae (true mice and rats, gerbils, spiny mice, crested rats), Nesomyidae (climbing mice, rock mice, with-tailed rats, Malagasy rats and mice), Platacanthomyidae (e.g., spiny dormice), and Spalacidae (e.g., mole rates, bamboo rats, and zokors). In some embodiments, the genetically modified rodent is selected from a true mouse or rat (family Muridae), a gerbil, a spiny mouse, and a crested rat. In some embodiments, the non-human animal is a mouse.

In some embodiments, the animal is a mouse of a C57BL strain selected from C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, and C57BL/Ola. In some embodiments, the mouse is a 129 strain selected from the group consisting of a strain that is 129P1, 129P2, 129P3, 129X1, 129S1 (e.g., 129S1/SV, 129S1/SvIm), 129S2, 129S4, 129S5, 129S9/SvEvH, 129S6 (129/SvEvTac), 129S7, 129S8, 129T1, 129T2. These mice are described, e.g., in Festing et al., Revised nomenclature for strain 129 mice, Mammalian Genome 10:836 (1999); Auerbach et al., Establishment and Chimera Analysis of 129/SvEv- and C57BL/6-Derived Mouse Embryonic Stem Cell Lines (2000), both of which are incorporated herein by reference in the entirety. In some embodiments, the genetically modified mouse is a mix of the 129 strain and the C57BL/6 strain. In some embodiments, the mouse is a mix of the 129 strains, or a mix of the BL/6 strains. In some embodiments, the mouse is a BALB strain, e.g., BALB/c strain. In some embodiments, the mouse is a mix of a BALB strain and another strain. In some embodiments, the mouse is from a hybrid line (e.g., 50% BALB/c-50% 12954/Sv; or 50% C57BL/6-50% 129). In some embodiments, the non-human animal is a rodent. In some embodiments, the non-human animal is a mouse having a BALB/c, A, A/He, A/J, A/WySN, AKR, AKR/A, AKR/J, AKR/N, TA1, TA2, RF, SWR, C3H, C57BR, SJL, C57L, DBA/2, KM, NIH, ICR, CFW, FACA, C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL (C57BL/10Cr and C57BL/Ola), C58, CBA/Br, CBA/Ca, CBA/J, CBA/st, or CBA/H background.

In one aspect, the non-human animal is a mammal. In one aspect, the non-human animal is a small mammal, e.g., a jerboa. In one embodiment, the genetically humanized non-human animal is a rodent. In one embodiment, the rodent is selected from the group consisting of mice, rats and hamsters. In one embodiment, the rodent is selected from the murine family. In one embodiment, the genetically modified animal is selected from a group consisting of hamsteridae (e.g., mouse-like hamsters), hamsteridae (e.g., hamsters, New World rats and mice, voles), murine superfamily (e.g., true mouse and rats, gerbils, spiny rats, and crested rats), Falkomuridae (e.g., climbing mice, rock mice, tailed rats, Madagascar rats and mice), Dormocidae (e.g., spiny dormouse) and Moleidae (e.g., mole rats, bamboo rats, and zokors) families. In a specific embodiment, the genetically modified rodent is selected from the group consisting of true mice or rats (Muridae), gerbils, spiny rats and crested rats. In one embodiment, the genetically modified mouse is from a member of the family Muridae. In one embodiment, the animal is a rodent. In a specific embodiment, the rodent is selected from mice and rats. In one embodiment, the non-human animal is a mouse.

In some embodiments, the animal is a rat. The rat can be selected from a Wistar rat, an LEA strain, a Sprague Dawley strain, a Fischer strain, F344, F6, and Dark Agouti. In some embodiments, the rat strain is a mix of two or more strains selected from the group consisting of Wistar, LEA, Sprague Dawley, Fischer, F344, F6, and Dark Agouti.

The animal can have one or more other genetic modifications, and/or other modifications, that are suitable for the particular purpose for which the humanized NKP46 animal is made. For example, suitable mice for maintaining a xenograft (e.g., a human cancer or tumor), can have one or more modifications that compromise, inactivate, or destroy the immune system of the non-human animal in whole or in part. Compromise, inactivation, or destruction of the immune system of the non-human animal can include, for example, destruction of hematopoietic cells and/or immune cells by chemical means (e.g., administering a toxin), physical means (e.g., irradiating the animal), and/or genetic modification (e.g., knocking out one or more genes). Non-limiting examples of such mice include, e.g., NOD mice, SCID mice, NOD/SCID mice, IL2Rγ knockout mice, NOD/SCID/γc^{null} mice (Ito, M. et al., NOD/SCID/γc^{null} mouse: an excellent recipient mouse model for engraftment of human cells, Blood 100 (9): 3175-3182, 2002), nude mice, and Rag1 and/or Rag2 knockout mice. These mice can optionally be irradiated, or otherwise treated to destroy one or more immune cell type. Thus, in various embodiments, a genetically modified mouse is provided that can include a humanization of at least a portion of an endogenous non-human NKP46 locus, and further comprises a modification that compromises, inactivates, or destroys the immune system (or one or more cell types of the immune system) of the non-human animal in whole or in part. In some embodiments, modification is, e.g., selected from the group consisting of a modification that results in NOD mice, SCID mice, NOD/SCID mice, IL-2Ry knockout mice, NOD/SCID/γc^{null} mice, nude mice, Rag1 and/or Rag2 knockout mice, NOD-Prkdc^{scid} IL-2rγ^{null} mice, NOD-Rag1^{-/-}-IL2rg^{-/-} (NRG) mice, Rag2^{-/-}-IL2rg^{-/-} (RG) mice, and a combination thereof. These genetically modified animals are described, e.g., in US20150106961, which is incorporated herein by reference in its entirety. In some embodiments, the mouse can include a replacement of all or part of mature NKP46 coding sequence with human mature NKP46 coding sequence.

Genetically modified non-human animals can comprise a modification at an endogenous non-human NKP46 locus. In some embodiments, the modification can comprise a human nucleic acid sequence encoding at least a portion of a mature NKP46 protein (e.g., at least 10%, 20%, 30%, 40%, 50%, 60%, 70%, 80%, 90%, 95%, 96%, 97%, 98%, or 99% identical to the mature NKP46 protein sequence). Although genetically modified cells are also provided that can comprise the modifications described herein (e.g., ES cells, somatic cells), in many embodiments, the genetically modified non-human animals comprise the modification of the endogenous NKP46 locus in the germline of the animal.

Genetically modified animals can express a human NKP46 and/or a chimeric (e.g., humanized) NKP46 from endogenous mouse loci, wherein the endogenous mouse NKP46 gene has been replaced with a human NKP46 gene and/or a nucleotide sequence that encodes a region of human NKP46 sequence or an amino acid sequence that is at least 10%, 20%, 30%, 40%, 50%, 60%, 70&, 80%, 90%, 95%, 96%, 97%, 98%, or 99% identical to the human NKP46 sequence. In various embodiments, an endogenous non-human NKP46 locus is modified in whole or in part to comprise human nucleic acid sequence encoding at least one protein-coding sequence of a mature NKP46 protein.

In some embodiments, the genetically modified mice can express the human NKP46 and/or chimeric NKP46 (e.g., humanized NKP46) from endogenous loci that are under control of mouse promoters and/or mouse regulatory elements. The replacement(s) at the endogenous mouse loci provide non-human animals that express human NKP46 or chimeric NKP46 (e.g., humanized NKP46) in appropriate cell types and in a manner that does not result in the potential pathologies observed in some other transgenic mice known in the art. The human NKP46 or the chimeric NKP46 (e.g., humanized NKP46) expressed in animal can maintain one or more functions of the wild-type mouse or human NKP46 in the animal. For example, the expressed NKP46 can activate NK cells. Furthermore, in some embodiments, the animal does not express endogenous NKP46. In some embodiments, the animal expresses a decreased level of endogenous NKP46 as compared to NKP46 expression level in a wild-type animal. As used herein, the term “endogenous NKP46” refers to NKP46 protein that is expressed from an endogenous NKP46 nucleotide sequence of the non-human animal (e.g., mouse) before any genetic modification.

The genome of the animal can comprise a sequence encoding an amino acid sequence that is at least 70%, 75%, 80%, 85%, 90%, 95%, 99%, or 100% identical to human NKP46 (NP_004820.2; SEQ ID NO: 2). In some embodiments, the genome comprises a sequence encoding an amino acid sequence that is at least 70%, 75%, 80%, 85%, 90%, 95%, 99%, or 100% identical to SEQ ID NO: 7 or 8.

The genome of the genetically modified animal can comprise a replacement at an endogenous NKP46 gene locus of a sequence encoding a region of endogenous NKP46 with a sequence encoding a corresponding region of human NKP46. In some embodiments, the sequence that is replaced is any sequence within the endogenous NKP46 gene locus, e.g., exon 1, exon 2, exon 3, exon 4, exon 5, exon 6, exon 7, 5′-UTR, 3′-UTR, intron 1, intron 2, intron 3, intron 4, intron 5, intron 6, or any combination thereof. In some embodiments, the sequence that is replaced is within the regulatory region of the endogenous NKP46 gene. In some embodiments, the sequence that is replaced is a portion of exon 1, exons 2-6, and a portion of exon 7, of an endogenous mouse NKP46 gene locus.

The genetically modified animal can have one or more cells expressing a human or chimeric NKP46 (e.g., humanized NKP46) having, from N-terminus to C-terminus, a signal peptide, an extracellular region, a transmembrane region, and a cytoplasmic region. In some embodiments, the signal peptide comprises a sequence that is at least 50%, 60%, 70%, 80%, 90%, 95%, 99% identical to the signal peptide of human NKP46. In some embodiments, the signal peptide of the humanized NKP46 has a sequence that has at least 1, 2, 3, 4, 5, 6, 7, 8, 9, 10, 11, 12, 13, 14, 15, 16, 17, 18, 19, 20, or 21 amino acids (e.g., contiguously or non-contiguously) that are identical to the signal peptide of human NKP46. In some embodiments, the extracellular region comprises a sequence that is at least 50%, 60%, 70%, 80%, 90%, 95%, 99% identical to the extracellular region of human NKP46. In some embodiments, the extracellular region of the humanized NKP46 has a sequence that has at least 10, 20, 30, 40, 50, 60, 70, 80, 90, 100, 110, 120, 130, 140, 150, 160, 170, 180, 190, 200, 210, 220, 230, 231, 232, 233, 234, 235, 236, or 237 amino acids (e.g., contiguously or non-contiguously) that are identical to the extracellular region of human NKP46. In some embodiments, the extracellular region of the humanized NKP46 has a sequence that has at least 1, 2, 3, 4, or 5 amino acids (contiguously or non-contiguously) that are identical to the C-terminal 1-5 amino acids in the extracellular region of endogenous NKP46 (e.g., mouse NKP46). In some embodiments, the extracellular region described herein includes the signal peptide. In some embodiments, the extracellular region described herein does not include the signal peptide. Because human NKP46 and non-human NKP46 (e.g., mouse NKP46) sequences, in many cases, are different, antibodies that bind to human NKP46 will not necessarily have the same binding affinity with non-human NKP46 or have the same effects to non-human NKP46. Therefore, the genetically modified animal having a human or a humanized extracellular region can be used to better evaluate the effects of anti-human NKP46 antibodies in an animal model.

In some embodiments, the transmembrane comprises a sequence that is at least 50%, 60%, 70%, 80%, 90%, 95%, 99% identical to the transmembrane region of endogenous NKP46 (e.g., amino acids 256-273 of SEQ ID NO: 1). In some embodiments, the transmembrane region of the humanized NKP46 has a sequence that has at least 1, 2, 3, 4, 5, 6, 7, 8, 9, 10, 11, 12, 13, 14, 15, 16, 17, or 18 amino acids (contiguously or non-contiguously) that are identical to the transmembrane region of endogenous NKP46 (e.g., mouse NKP46). In some embodiments, the cytoplasmic comprises a sequence that is at least 50%, 60%, 70%, 80%, 90%, 95%, 99% identical to the cytoplasmic of endogenous NKP46 (e.g., amino acids 274-325 of SEQ ID NO: 1). In some embodiments, the cytoplasmic region of the humanized NKP46 has a sequence that has at least 10, 15, 20, 25, 30, 35, 40, 45, 50, 51, or 52 amino acids (contiguously or non-contiguously) that are identical to the cytoplasmic region of endogenous NKP46 (e.g., mouse NKP46).

In some embodiments, the entire transmembrane region and the entire cytoplasmic region of the humanized NKP46 described herein are derived from endogenous sequence.

In some embodiments, the genome of the genetically modified animal comprises a sequence encoding an amino acid sequence that corresponds to a portion or the entire sequence of exon 1, exon 2, exon 3, exon 4, exon 5, exon 6, and/or exon 7 of human NKP46; a portion or the entire sequence of the extracellular region, and/or a portion or the entire sequence of the signal peptide of human NKP46; or a portion or the entire sequence of amino acids 1-253, 2-253, or 22-253 of SEQ ID NO: 2.

In some embodiments, the genome of the genetically modified animal comprises a portion of exon 1, exons 2-6, and a portion of exon 7 of human NKP46 gene. In some embodiments, the portion of exon 1 includes at least 5, 10, 15, 20, 25, 28, 29, 30, 31, 32, 33, 34, 35, 40, 45, 50, 55, 60, 65, 70, 71, 72, 73, or 74 nucleotides. In some embodiments, the portion of exon 1 includes 31 nucleotides. In some embodiments, the portion of exon 1 includes a nucleotide of at least 20 bp. In some embodiments, the portion of exon 7 includes at least 1, 2, 3, 4, 5, 6, 7, 8, 9, 10, 11, 12, 13, 14, 15, 20, 25, 26, 27, 28, 29, 30, 40, 50, 55, 56, 60, 70, 80, 90, 100, 150, 200, 250, 300, 350, 360, 370, 380, or 384 nucleotides. In some embodiments, the portion of exon 7 includes 26 nucleotides. In some embodiments, the portion of exon 7 includes a nucleotide of at least 10 bp.

In some embodiments, the non-human animal can have, at an endogenous NKP46 gene locus, a nucleotide sequence encoding a chimeric human/non-human NKP46 polypeptide, wherein a human portion of the chimeric human/non-human NKP46 polypeptide comprises the entire human NKP46 signal peptide and all or a portion of the human NKP46 extracellular region, and wherein the animal expresses a functional NKP46 on a surface of a cell of the animal. The human portion of the chimeric human/non-human NKP46 polypeptide can comprise an amino acid sequence encoded by a portion of exon 1, exons 2-6, and/or a portion of exon 7 of human NKP46. In some embodiments, the human portion of the chimeric human/non-human NKP46 polypeptide can comprise a sequence that is at least 80%, 85%, 90%, 95%, or 99% identical to amino acids 2-253 of SEQ ID NO: 2. In some embodiments, the transmembrane region includes a sequence corresponding to the entire or part of amino acids 256-273 of SEQ ID NO: 1. In some embodiments, the cytoplasmic region includes a sequence corresponding to the entire or part of amino acids 274-325 of SEQ ID NO: 1. In some embodiments, the chimeric human/non-human NKP46 polypeptide comprises a signal peptide, which includes a sequence corresponding to the entire or part of amino acids 1-21 of SEQ ID NO: 2.

In some embodiments, the non-human portion of the chimeric human/non-human NKP46 polypeptide comprises the entire transmembrane region and/or the entire cytoplasmic region of an endogenous non-human NKP46 polypeptide.

Furthermore, the genetically modified animal can be heterozygous with respect to the replacement at the endogenous NKP46 locus, or homozygous with respect to the replacement at the endogenous NKP46 locus.

In some embodiments, the humanized NKP46 locus lacks a human NKP46 gene 5′-UTR. In some embodiment, the humanized NKP46 locus comprises an endogenous (e.g., mouse) 5′-UTR. In some embodiments, the humanization comprises an endogenous (e.g., mouse) 3′-UTR. In appropriate cases, it may be reasonable to presume that the mouse and human NKP46 genes appear to be similarly regulated based on the similarity of their 5′-flanking sequence. As shown in the present disclosure, humanized NKP46 mice that comprise a replacement at an endogenous mouse NKP46 locus, which retain mouse regulatory elements but comprise a humanization of NKP46 encoding sequence, do not exhibit pathologies. Both genetically modified mice that are heterozygous or homozygous for humanized NKP46 are grossly normal.

The present disclosure further relates to a non-human mammal generated through the method mentioned above. In some embodiments, the genome thereof contains human gene(s).

In some embodiments, the non-human mammal is a rodent, and preferably, the non-human mammal is a mouse.

In some embodiments, the non-human mammal expresses a protein encoded by a humanized NKP46 gene.

In addition, the present disclosure also relates to a tumor bearing non-human mammal model, characterized in that the non-human mammal model is obtained through the methods as described herein. In some embodiments, the non-human mammal is a rodent (e.g., a mouse).

The present disclosure further relates to a cell or cell line, or a primary cell culture thereof derived from the non-human mammal or an offspring thereof, or the tumor bearing non-human mammal; the tissue, organ or a culture thereof derived from the non-human mammal or an offspring thereof, or the tumor bearing non-human mammal; and the tumor tissue derived from the non-human mammal or an offspring thereof when it bears a tumor, or the tumor bearing non-human mammal.

The present disclosure also provides non-human mammals produced by any of the methods described herein. In some embodiments, a non-human mammal is provided; and the genetically modified animal contains the DNA encoding human or humanized NKP46 in the genome of the animal.

In some embodiments, the non-human mammal comprises the genetic construct as described herein (e.g., gene construct as shown in FIGS. 2, 3, 4, and 5). In some embodiments, a non-human mammal expressing human or humanized NKP46 is provided. In some embodiments, the tissue-specific expression of human or humanized NKP46 protein is provided.

In some embodiments, the expression of human or humanized NKP46 in a genetically modified animal is controllable, as by the addition of a specific inducer or repressor substance. In some embodiments, the specific inducer is selected from Tet-Off System/Tet-On System, or Tamoxifen System.

Non-human mammals can be any non-human animal known in the art and which can be used in the methods as described herein. Preferred non-human mammals are mammals, (e.g., rodents). In some embodiments, the non-human mammal is a mouse.

Genetic, molecular and behavioral analyses for the non-human mammals described above can be performed. The present disclosure also relates to the progeny produced by the non-human mammal provided by the present disclosure mated with the same or other genotypes.

The present disclosure also provides a cell line or primary cell culture derived from the non-human mammal or a progeny thereof. A model based on cell culture can be prepared, for example, by the following methods. Cell cultures can be obtained by way of isolation from a non-human mammal, alternatively cells can be obtained from the cell culture established using the same constructs and the standard cell transfection techniques. The integration of genetic constructs containing DNA sequences encoding human NKP46 protein can be detected by a variety of methods.

There are many analytical methods that can be used to detect exogenous DNA, including methods at the level of nucleic acid (including the mRNA quantification approaches using reverse transcriptase polymerase chain reaction (RT-PCR) or Southern blotting, and in situ hybridization) and methods at the protein level (including histochemistry, immunoblot analysis and in vitro binding studies). In addition, the expression level of the gene of interest can be quantified by ELISA techniques well known to those skilled in the art. Many standard analysis methods can be used to complete quantitative measurements. For example, transcription levels can be measured using RT-PCR and hybridization methods including RNase protection, Southern blot analysis, RNA dot analysis (RNAdot) analysis. Immunohistochemical staining, flow cytometry, Western blot analysis can also be used to assess the presence of human or humanized NKP46 protein.

In another aspect, the disclosure also provides a genetically-modified, non-human animal whose genome comprise a disruption in the animal's endogenous NKP46 gene, wherein the disruption of the endogenous NKP46 gene comprises deletion of exon 1, exon 2, exon 3, exon 4, exon 5, exon 6, and/or exon 7, or part thereof of the endogenous NKP46 gene.

In some embodiments, the disruption of the endogenous NKP46 gene comprises deletion of one or more exons or part of exons selected from the group consisting of exon 1, exon 2, exon 3, exon 4, exon 5, exon 6, and exon 7 of the endogenous NKP46 gene.

In some embodiments, the disruption of the endogenous NKP46 gene further comprises deletion of one or more introns or part of introns selected from the group consisting of intron 1, intron 2, intron 3, intron 4, intron 5, and intron 6 of the endogenous NKP46 gene.

In some embodiments, wherein the deletion can comprise deleting at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 300, 400, 500, 600, 700, 800, 900, 1000, 1100, 1200, 1300, 1400, 1500, 1600, 1700, 1800, 1900, 2000, 2500, 3000, 5000, 6000, 7000, or more nucleotides.

In some embodiments, the disruption of the endogenous NKP46 gene comprises the deletion of at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 10, 220, 230, 240, 250, 260, 270, 280, 290, or 300 nucleotides of exon 1, exon 2, exon 3, exon 4, exon 5, exon 6, and/or exon 7 (e.g., deletion of at least 10 nucleotides from exon 1, exons 2-6, and at least 5 nucleotides from exon 7).

### Vectors

The present disclosure relates to a targeting vector, comprising: a) a DNA fragment homologous to the 5′ end of a region to be altered (5′ arm), which is selected from the NKP46 gene genomic DNAs in the length of 100 to 10,000 nucleotides; b) a desired/donor DNA sequence encoding a donor region; and c) a second DNA fragment homologous to the 3′ end of the region to be altered (3′ arm), which is selected from the NKP46 gene genomic DNAs in the length of 100 to 10,000 nucleotides.

In some embodiments, a) the DNA fragment homologous to the 5′ end of a conversion region to be altered (5′ arm) is selected from the nucleotide sequences that have at least 90% homology to the NCBI accession number NC_000073.7; c) the DNA fragment homologous to the 3′ end of the region to be altered (3′ arm) is selected from the nucleotide sequences that have at least 90% homology to the NCBI accession number NC_000073.7.

In some embodiments, a) the DNA fragment homologous to the 5′ end of a region to be altered (5′ arm) is selected from the nucleotides from the position 4336716 to the position 4340750 of the NCBI accession number NC 000073.7; c) the DNA fragment homologous to the 3′ end of the region to be altered (3′ arm) is selected from the nucleotides from the position 4348522 to the position 4352569 of the NCBI accession number NC 000073.7.

In some embodiments, a) the DNA fragment homologous to the 5′ end of a region to be altered (5′ arm) is selected from the nucleotides from the position 4339331 to the position 4340750 of the NCBI accession number NC_000073.7; c) the DNA fragment homologous to the 3′ end of the region to be altered (3′ arm) is selected from the nucleotides from the position 4347485 to the position 4348999 of the NCBI accession number NC_000073.7.

In some embodiments, the length of the selected genomic nucleotide sequence in the targeting vector can be more than about 3 kb, about 3.5 kb, about 4 kb, about 4.5 kb, about 5 kb, about 5.5 kb, about 6 kb, about 6.5 kb, about 7 kb, about 7.5 kb, or about 8 kb.

In some embodiments, the region to be altered is exon 1, exon 2, exon 3, exon 4, exon 5, exon 6, and/or exon 7 of NKP46 gene (e.g., a portion of exon 1, exons 2-6, and a portion of exon 7 of mouse NKP46 gene).

The targeting vector can further include one or more selectable markers, e.g., positive or negative selectable markers. In some embodiments, the positive selectable marker is a Neo gene or Neo cassette. In some embodiments, the negative selectable marker is a DTA gene.

In some embodiments, the sequence of the 5′ arm is shown in SEQ ID NO: 3; and the sequence of the 3′ arm is shown in SEQ ID NO: 4. In some embodiments, the sequence of the 5′ arm is shown in SEQ ID NO: 5; and the sequence of the 3′ arm is shown in SEQ ID NO: 6.

In some embodiments, the sequence is derived from human (e.g., 54906191-54912715 of NC 000019.10; or 44-799 of NM 004829.7). For example, the target region in the targeting vector is a part or entirety of the nucleotide sequence of a human NKP46 gene, preferably exon 1, exon 2, exon 3, exon 4, exon 5, exon 6, and/or exon 7 of the human NKP46 gene. In some embodiments, the nucleotide sequence of the humanized NKP46 gene encodes the entire or the part of human NKP46 protein with the NCBI accession number NP_004820.2 (SEQ ID NO: 2).

The disclosure also provides vectors for constructing a humanized animal model or a knock-out model. In some embodiments, the vectors comprise a sgRNA sequence, wherein the sgRNA sequence targets NKP46 gene, and the sgRNA is unique on the target sequence of the gene to be altered, and meets the sequence arrangement rule of 5′-NNN (20)-NGG3′ or 5′-CCN—N(20)-3′; and in some embodiments, the targeting site of the sgRNA in the mouse NKP46 gene is located on the exon 1, exon 2, exon 3, exon 4, exon 5, exon 6, exon 7, intron 1, intron 2, intron 3, intron 4, intron 5, intron 6, upstream of exon 1, or downstream of exon 7 of the mouse NKP46 gene.

In some embodiments, the targeting sequences are shown as SEQ ID NOs: 14, 15, 16, 17, 18, 19, 20, 21, 22, and 23. Thus, the disclosure provides sgRNA sequences for constructing a genetic modified animal model. In some embodiments, the oligonucleotide sgRNA sequences are set forth in SEQ ID NOs: 14 and 15. In some embodiments, the oligonucleotide sgRNA sequences are set forth in SEQ ID NOs: 16 and 18. In some embodiments, the oligonucleotide sgRNA sequences are set forth in SEQ ID NOs: 17 and 19. In some embodiments, the oligonucleotide sgRNA sequences are set forth in SEQ ID NOs: 20 and 22. In some embodiments, the oligonucleotide sgRNA sequences are set forth in SEQ ID NOs: 21 and 23.

In some embodiments, the disclosure relates to a plasmid construct (e.g., pT7-sgRNA) including the sgRNA sequence, and/or a cell including the construct.

The disclosure also relates to a cell comprising the targeting vectors as described above.

In addition, the present disclosure further relates to a non-human mammalian cell, having any one of the foregoing targeting vectors, and one or more in vitro transcripts of the construct as described herein. In some embodiments, the cell includes Cas9 mRNA or an in vitro transcript thereof.

In some embodiments, the genes in the cell are heterozygous. In some embodiments, the genes in the cell are homozygous.

In some embodiments, the non-human mammalian cell is a mouse cell. In some embodiments, the cell is a fertilized egg cell. In some embodiments, the cell is an embryonic stem cell.

### Methods of Making Genetically Modified Animals

Genetically modified animals can be made by several techniques that are known in the art, including, e.g., nonhomologous end-joining (NHEJ), homologous recombination (HR), zinc finger nucleases (ZFNs), transcription activator-like effector-based nucleases (TALEN), and the clustered regularly interspaced short palindromic repeats (CRISPR)-Cas system. In some embodiments, homologous recombination is used. In some embodiments, CRISPR-Cas9 genome editing is used to generate genetically modified animals. Many of these genome editing techniques are known in the art, and is described, e.g., in Yin et al., “Delivery technologies for genome editing,” Nature Reviews Drug Discovery 16.6 (2017): 387-399, which is incorporated by reference in its entirety. Many other methods are also provided and can be used in genome editing, e.g., micro-injecting a genetically modified nucleus into an enucleated oocyte, and fusing an enucleated oocyte with another genetically modified cell.

Thus, in some embodiments, the disclosure provides replacing in at least one cell of the animal, at an endogenous NKP46 gene locus, a sequence encoding a region of an endogenous NKP46 with a sequence encoding a corresponding region of human or chimeric NKP46. In some embodiments, the replacement occurs in a germ cell, a somatic cell, a blastocyst, or a fibroblast, etc. The nucleus of a somatic cell or the fibroblast can be inserted into an enucleated oocyte.

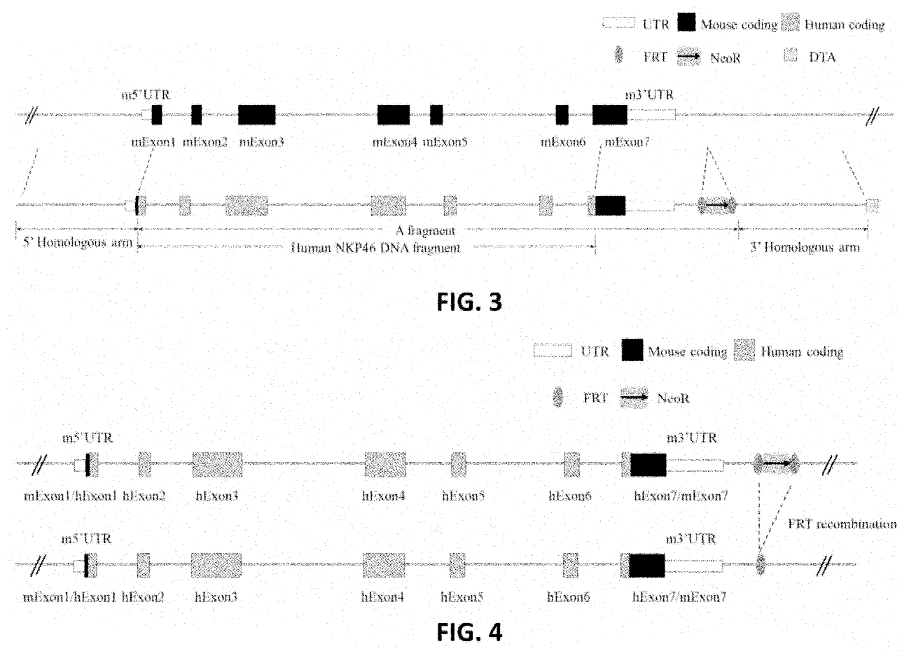

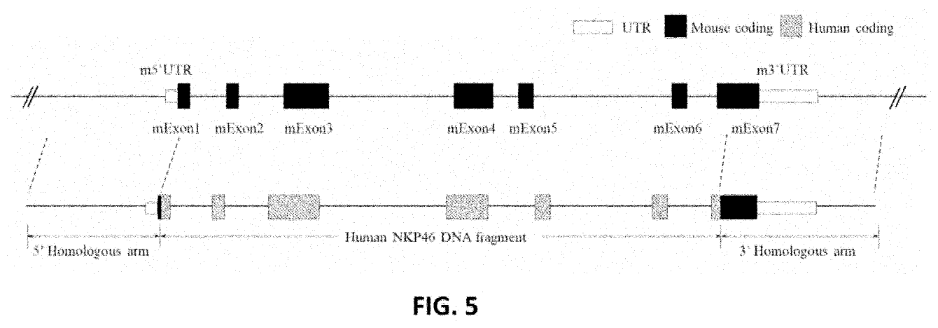

FIG. 3 and FIG. 5 show humanization strategies for a mouse NKP46 locus. The targeting strategies involve a vector comprising a 5′ homologous arm, a human NKP46 gene fragment, and a 3′ homologous arm. The process can involve replacing endogenous NKP46 sequence with human sequence by homologous recombination. In some embodiments, the cleavage at the upstream and the downstream of the target site (e.g., by zinc finger nucleases, TALEN or CRISPR) can result in DNA double strands break, and the homologous recombination is used to replace endogenous NKP46 sequence with human NKP46 sequence.

Thus, in some embodiments, the methods for making a genetically modified, humanized animal, can include the step of replacing at an endogenous NKP46 locus (or site), a nucleic acid sequence encoding a region of endogenous NKP46 with a sequence encoding a corresponding region of human NKP46. The sequence can include a region (e.g., a part or the entire region) of exon 1, exon 2, exon 3, exon 4, exon 5, exon 6, and/or exon 7 of a human NKP46 gene. In some embodiments, the sequence includes a portion of exon 1, exons 2-6, and a portion of exon 7 of a human NKP46 gene (e.g., nucleic acids 44-799 of NM 004829.7). In some embodiments, the region includes the signal peptide of human NKP46 (e.g., amino acids 1-21 or 2-21 of SEQ ID NO: 2), and/or the extracellular region of human NKP46 (e.g., amino acids 22-253 or 22-258 of SEQ ID NO: 2). In some embodiments, the endogenous NKP46 locus is exon 1, exon 2, exon 3, exon 4, exon 5, exon 6, and/or exon 7 of mouse NKP46. In some embodiments, the sequence includes a portion of exon 1 and a portion of exon 7 of mouse NKP46 gene (e.g., nucleic acids 1-28 and 785-1463 of NM_010746.3).

In some embodiments, the methods of modifying a NKP46 locus of a mouse to express a chimeric human/mouse NKP46 peptide can include the steps of replacing at the endogenous mouse NKP46 locus a nucleotide sequence encoding a mouse NKP46 with a nucleotide sequence encoding a human NKP46, thereby generating a sequence encoding a chimeric human/mouse NKP46.

In some embodiments, the nucleotide sequence encoding the chimeric human/mouse NKP46 can include a first nucleotide sequence encoding the signal peptide and all or a portion of the extracellular region of human NKP46; and a second nucleotide sequence encoding the transmembrane region and the cytoplasmic region of mouse NKP46, optionally the C-terminal 1, 2, 3, 4, or 5 amino acids in the extracellular region of mouse NKP46.

In some embodiments, the nucleotide sequences as described herein do not overlap with each other (e.g., the first nucleotide sequence, the second nucleotide sequence, and/or the third nucleotide sequence do not overlap). In some embodiments, the amino acid sequences as described herein do not overlap with each other.

The present disclosure further provides a method for establishing a NKP46 gene humanized animal model, involving the following steps:
(a) providing the cell (e.g. a fertilized egg cell) based on the methods described herein;
(b) culturing the cell in a liquid culture medium;
(c) transplanting the cultured cell to the fallopian tube or uterus of the recipient female non-human mammal, allowing the cell to develop in the uterus of the female non-human mammal;
(d) identifying the germline transmission in the offspring genetically modified humanized non-human mammal of the pregnant female in step (c).

In some embodiments, the non-human mammal in the foregoing method is a mouse (e.g., a C57BL/6 mouse).

In some embodiments, the non-human mammal in step (c) is a female with pseudopregnancy (or false pregnancy).

In some embodiments, the fertilized eggs for the methods described above are C57BL/6 fertilized eggs. Other fertilized eggs that can also be used in the methods as described herein include, but are not limited to, FVB/N fertilized eggs, BALB/c fertilized eggs, DBA/1 fertilized eggs and DBA/2 fertilized eggs.

Fertilized eggs can come from any non-human animal, e.g., any non-human animal as described herein. In some embodiments, the fertilized egg cells are derived from rodents. The genetic construct can be introduced into a fertilized egg by microinjection of DNA. For example, by way of culturing a fertilized egg after microinjection, a cultured fertilized egg can be transferred to a false pregnant non-human animal, which then gives birth of a non-human mammal, so as to generate the non-human mammal mentioned in the methods described above.

In some embodiments, methods of making the genetically modified animal comprises modifying the coding frame of the non-human animal's NKP46 gene, e.g., by inserting a nucleotide sequence (e.g., DNA or cDNA sequence) encoding human or humanized NKP46 protein, e.g., immediately after the endogenous regulatory element of the non-human animal's NKP46 gene. For example, one or more functional region sequences of the non-human animal's NKP46 gene can be knocked out, or inserted with a sequence, such that the non-human animal cannot express or expresses a decreased level of endogenous NKP46 protein. In some embodiments, the coding frame of the modified non-human animal's NKP46 gene can be all or part of the nucleotide sequence from exon 1 to exon 7 of the non-human animal's NKP46 gene.

In some embodiments, methods of making the genetically modified animal comprises inserting a nucleotide sequence encoding human or humanized NKP46 protein and/or an auxiliary sequence after the endogenous regulatory element of the non-human animal's NKP46 gene. In some embodiments, the auxiliary sequence can be a stop codon, such that the NKP46 gene humanized animal model can express human or humanized NKP46 protein in vivo, but does not express non-human animal's NKP46 protein. In some embodiments, the auxiliary sequence includes WPRE (WHP Posttranscriptional Response Element), loxP, and/or polyA.

In some embodiments, the insertion refers to placing a target fragment directly between two adjacent bases without deleting nucleotides. For example, the target fragment can be a human NKP46 gene, a humanized NKP46 gene, a nucleotide sequence encoding a human or humanized NKP46 protein, or a nucleotide sequence obtained by splicing human NKP46 and non-human NKP46 genes. In some embodiments, the target fragment can also be a partial nucleotide sequence of the human NKP46 gene. Preferably, exon x+1 to exon 7 of the human NKP46 gene can be inserted adjacent to exon x of the NKP46 gene of non-human animals. For example, exon 2 to exon 7 of the human NKP46 gene can be inserted adjacent to exon 1 of the NKP46 gene of non-human animals; exon 3 to exon 7 of the human NKP46 gene can be inserted adjacent to exon 2 of the NKP46 gene of non-human animals; exon 4 to exon 7 of the human NKP46 gene can be inserted adjacent to exon 3 of the NKP46 gene of non-human animals; or exon 5 to exon 7 of human NKP46 gene can be inserted adjacent to exon 4 of the NKP46 gene of non-human animals.

In some embodiments, the method for making the genetically modified animal comprises:
(1) providing a plasmid comprising a human NKP46 gene fragment, flanked by a 5′ homologous arm and a 3′ homologous arm, wherein the 5′ and 3′ homologous arms target an endogenous NKP46 gene;
(2) providing one or more small guide RNAs (sgRNAs) that target the endogenous NKP46 gene;
(3) modifying genome of a fertilized egg or an embryonic stem cell by using the plasmid of step (1), the sgRNAs of step (2), and Cas9;
(4) transplanting the fertilized egg obtained in step (3) into the oviduct of a pseudopregnant female mouse or transplanting the embryonic stem cell obtained in step (3) into a blastocyst which is then transplanted into the oviduct of a pseudopregnant female mouse to produce a child mouse that functionally expresses a humanized NKP46 protein; and
(5) mating the child mouse obtained in step (2) to obtain a homozygote mouse,

In some embodiments, the fertilized egg is modified by CRISPR with sgRNAs that target a 5′-terminal targeting site and a 3′-terminal targeting site.

In some embodiments, the sequence encoding the humanized NKP46 protein is operably linked to an endogenous regulatory element at the endogenous NKP46 gene locus.

In some embodiments, the genetically-modified animal does not express an endogenous NKP46 protein.

In some embodiments, the method for making the genetically modified animal comprises:
(1) providing a plasmid comprising a human or chimeric NKP46 gene fragment, flanked by a 5′ homologous arm and a 3′ homologous arm, wherein the 5′ and 3′ homologous arms target an endogenous NKP46 gene;
(2) providing one or more small guide RNAs (sgRNAs) that target the endogenous NKP46 gene; and
(3) modifying genome of a fertilized egg or an embryonic stem cell by inserting the human or chimeric NKP46 gene fragment into the genome.

### Methods of Using Genetically Modified Animals

Replacement of non-human genes in a non-human animal with homologous or orthologous human genes or human sequences, at the endogenous non-human locus and under control of endogenous promoters and/or regulatory elements, can result in a non-human animal with qualities and characteristics that may be substantially different from a typical knockout-plus-transgene animal. In the typical knockout-plus-transgene animal, an endogenous locus is removed or damaged and a fully human transgene is inserted into the animal's genome and presumably integrates at random into the genome. Typically, the location of the integrated transgene is unknown; expression of the human protein is measured by transcription of the human gene and/or protein assay and/or functional assay. Inclusion in the human transgene of upstream and/or downstream human sequences are apparently presumed to be sufficient to provide suitable support for expression and/or regulation of the transgene.

In some cases, the transgene with human regulatory elements expresses in a manner that is unphysiological or otherwise unsatisfactory, and can be actually detrimental to the animal. The disclosure demonstrates that a replacement with human sequence at an endogenous locus under control of endogenous regulatory elements provides a physiologically appropriate expression pattern and level that results in a useful humanized animal whose physiology with respect to the replaced gene are meaningful and appropriate in the context of the humanized animal's physiology.

Genetically modified animals that express human or humanized NKP46 protein, e.g., in a physiologically appropriate manner, provide a variety of uses that include, but are not limited to, developing therapeutics for human diseases and disorders, and assessing the toxicity and/or the efficacy of these human therapeutics in the animal models.

In various aspects, genetically modified animals are provided that express human or humanized NKP46, which are useful for testing therapeutic agents that can decrease or block the interaction between the interaction between NKP46 and anti-human NKP46 antibodies, testing whether an therapeutic agent can increase or decrease the immune response, and/or determining whether an agent is an NKP46 agonist or antagonist. The genetically modified animals can be, e.g., an animal model of a human disease, e.g., the disease is induced genetically (a knock-in or knockout). In various embodiments, the genetically modified non-human animals further comprise an impaired immune system, e.g., a non-human animal genetically modified to sustain or maintain a human xenograft, e.g., a human solid tumor (e.g., lung cancer) or a blood cell tumor (e.g., a lymphocyte tumor, a B or T cell tumor).

In some embodiments, the therapeutic agent (e.g., an anti-NKP46 antibody) described herein can bind to NKP46 expressed on the surface of NK cells and induce activation of the NK cells, thereby treating cancer. In some embodiments, the therapeutic agent (e.g., an anti-NKP46 antibody) described herein can target NKP46 expressed on the surface of NK cells, to inhibit viral infection.

In some embodiments, the genetically modified animals can be used for determining effectiveness of a therapeutic agent (e.g., an anti-NKP46 antibody or a NKP46-targeting drug) for the treatment of cancer. In some embodiments, the methods involve administering the therapeutic agent (e.g., an anti-human NKP46 antibody or a NKP46-targeting drug) to the animal as described herein, wherein the animal has a cancer or tumor; and determining inhibitory effects of the therapeutic agent to the cancer or tumor. The inhibitory effects that can be determined include, e.g., a decrease of tumor size or tumor volume, a decrease of tumor growth, a reduction of the increase rate of tumor volume in a subject (e.g., as compared to the rate of increase in tumor volume in the same subject prior to treatment or in another subject without such treatment), a decrease in the risk of developing a metastasis or the risk of developing one or more additional metastasis, an increase of survival rate, and an increase of life expectancy, etc. The tumor volume in a subject can be determined by various methods, e.g., as determined by direct measurement, MRI or CT. In some embodiments, the anti-NKP46 antibody can directly target NK cells expressing NKP46, e.g., by inducing activation of NK cells to kill the cancer cells.

In some embodiments, the tumor comprises one or more cancer cells (e.g., human or mouse cancer cells) that are injected into the animal. In some embodiments, the anti-NKP46 antibody activates NKP46 signaling pathways. In some embodiments, the anti-NKP46 antibody does not activate NKP46 signaling pathways. In some embodiments, the anti-NKP46 antibody inhibits NKP46 signaling pathways.

In some embodiments, the genetically modified animals can be used for determining whether an anti-NKP46 antibody is a NKP46 agonist or antagonist. In some embodiments, the methods as described herein are also designed to determine the effects of the therapeutic agent (e.g., anti-NKP46 antibodies) on NKP46, e.g., whether the agent can activate NK cells, whether the agent can upregulate the immune response or downregulate immune response, and/or whether the agent can induce complement mediated cytotoxicity (CMC) or antibody dependent cellular cytotoxicity (ADCC). In some embodiments, the genetically modified animals can be used for determining the effective dosage of a therapeutic agent for treating a disease in the subject, e.g., cancer.

The inhibitory effects on tumors can also be determined by methods known in the art, e.g., measuring the tumor volume in the animal, and/or determining tumor (volume) inhibition rate (TGI_{TV}). The tumor growth inhibition rate can be calculated using the formula TGI_{TV} (%)-(1-TVt/TVc)×100, where TVt and TVc are the mean tumor volume (or weight) of treated and control groups.

In some embodiments, the therapeutic agent (e.g., an anti-NKP46 antibody or a NKP46-targeting drug) is designed for treating various cancers. As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells. Cancers that can be treated or diagnosed using the methods described herein include malignancies of the various organ systems, such as affecting lung, breast, thyroid, lymphoid, gastrointestinal, and genitourinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. In some embodiments, the agents described herein are designed for treating or diagnosing a carcinoma in a subject. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the cancer is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

In some embodiments, the cancer described herein is lymphoma, non-small cell lung cancer, cervical cancer, leukemia, ovarian cancer, nasopharyngeal cancer, breast cancer, endometrial cancer, colon cancer, rectal cancer, gastric cancer, bladder cancer, glioma, lung cancer, bronchial cancer, bone cancer, prostate cancer, pancreatic cancer, liver and bile duct cancer, esophageal cancer, kidney cancer, thyroid cancer, head and neck cancer, testicular cancer, glioblastoma, astrocytoma, melanoma, myeloproliferation abnormal syndromes, and sarcomas. In some embodiments, the leukemia is selected from acute lymphocytic (lymphoblastic) leukemia, acute myeloid leukemia, myeloid leukemia, chronic lymphocytic leukemia, multiple myeloma, plasma cell leukemia, and chronic myelogenous leukemia. In some embodiments, the lymphoma is selected from Hodgkin's lymphoma and non-Hodgkin's lymphoma, including B-cell lymphoma, diffuse large B-cell lymphoma, follicular lymphoma, mantle cell lymphoma, marginal zone B-cell lymphoma, T-cell lymphoma, and Waldenstrom macroglobulinemia. In some embodiments, the sarcoma is selected from the group consisting of osteosarcoma, Ewing sarcoma, leiomyosarcoma, synovial sarcoma, soft tissue sarcoma, angiosarcoma, liposarcoma, fibrosarcoma, rhabdomyosarcoma, and chondrosarcoma. In a specific embodiment, the tumor is non-small cell lung cancer, metastatic colorectal cancer, cervical cancer, ovarian cancer, nasopharyngeal cancer, gastric cancer, glioma.

In some embodiments, the cancer described herein is lung cancer, leukemia, colon cancer, head and neck cancer, kidney cancer, pancreatic cancer, or gastric cancer.

In some embodiments, the therapeutic agent (e.g., an anti-NKP46 antibody or a NKP46-targeting drug) is designed for treating various autoimmune diseases, including rheumatoid arthritis, Crohn's disease, systemic lupus erythematosus, ankylosing spondylitis, inflammatory bowel diseases (IBD), ulcerative colitis, or scleroderma. In some embodiments, the anti-NKP46 antibody is designed for treating various immune disorders, including allergy, asthma, and/or atopic dermatitis. Thus, the methods as described herein can be used to determine the effectiveness of an therapeutic agent (e.g., an anti-NKP46 antibody or a NKP46-targeting drug) in inhibiting immune response. In some embodiments, the immune disorders described herein is graft versus host disease (GVHD), psoriasis, allergy, asthma, myocarditis, nephritis, hepatitis, systemic lupus erythematosus, rheumatoid arthritis, scleroderma, hyperthyroidism, idiopathic thrombocytopenia purpura, autoimmune hemolytic anemia, ulcerative colitis, autoimmune liver disease, diabetes, pain or neurological disorders, etc. In some embodiments, the therapeutic agent (e.g., an anti-NKP46 antibody or a NKP46-targeting drug) is designed for treating various inflammations, e.g., viral inflammation. In some embodiments, the inflammation described herein includes both acute inflammation and chronic inflammation. Specifically, the inflammation includes but not limited to degenerative inflammation, exudative inflammation (e.g., serous inflammation, fibrinous inflammation, suppurative inflammation, hemorrhagic inflammation, necrotic inflammation, catarrhal inflammation), proliferative inflammation, specific inflammation (e.g., tuberculosis, syphilis, leprosy, or lymphogranuloma).

The present disclosure also provides methods of determining toxicity of an antibody (e.g., anti-NKP46 antibody). The methods involve administering the antibody to the animal as described herein. The animal is then evaluated for its weight change, red blood cell count, hematocrit, and/or hemoglobin. In some embodiments, the antibody can decrease the red blood cells (RBC), hematocrit, or hemoglobin by more than 20%, 30%, 40%, or 50%. In some embodiments, the animals can have a weight that is at least 5%, 10%, 20%, 30%, or 40% smaller than the weight of the control group (e.g., average weight of the animals that are not treated with the antibody).

The present disclosure also relates to the use of the animal model generated through the methods as described herein in the development of a product related to an immunization processes of human cells, the manufacturing of a human antibody, or the model system for a research in pharmacology, immunology, microbiology and medicine.

In some embodiments, the disclosure provides the use of the animal model generated through the methods as described herein in the production and utilization of an animal experimental disease model of an immunization processes involving human cells, the study on a pathogen, or the development of a new diagnostic strategy and/or a therapeutic strategy.

The disclosure also relates to the use of the animal model generated through the methods as described herein in the screening, verifying, evaluating or studying the NKP46 gene function, human NKP46 antibodies, drugs for human NKP46 targeting sites, the drugs or efficacies for human NKP46 targeting sites, the drugs for immune-related diseases and antitumor drugs.

In some embodiments, the disclosure provides a method to verify in vivo efficacy of TCR-T, CAR-T, and/or other immunotherapies (e.g., T-cell adoptive transfer therapies). For example, the methods include transplanting human tumor cells into the animal described herein, and applying human CAR-T to the animal with human tumor cells. Effectiveness of the CAR-T therapy can be determined and evaluated. In some embodiments, the animal is selected from the NKP46 gene humanized non-human animal prepared by the methods described herein, the NKP46 gene humanized non-human animal described herein, the double- or multi-humanized non-human animal generated by the methods described herein (or progeny thereof), a non-human animal expressing the human or humanized NKP46 protein, or the tumor-bearing or inflammatory animal models described herein. In some embodiments, the TCR-T, CAR-T, and/or other immunotherapies can treat the NKP46-associated diseases described herein. In some embodiments, the TCA-T, CAR-T, and/or other immunotherapies provides an evaluation method for treating the NKP46-associated diseases described herein.

Genetically Modified Animal Model with Two or More Human or Chimeric Genes

The present disclosure further relates to methods for generating genetically modified animal model with two or more human or chimeric genes. The animal can comprise a human or chimeric NKP46 gene and a sequence encoding an additional human or chimeric protein.

In some embodiments, the additional human or chimeric protein can be natural killer group 2D (NKG2D), epidermal growth factor receptor (EGFR), receptor tyrosine-protein kinase erbB-2 (HER2), cluster of differentiation 276 (B7H3), B-cell maturation antigen (BCMA), fibroblast activation protein alpha (FAP), C—X—C chemokine receptor type 4 (CXCR4), colony stimulating factor 2 (CSF2), tumor necrosis factor receptor 2 (TNFR2), interleukin 2 (IL-2), interleukin 15 (IL-15), interleukin 15 receptor, alpha subunit (IL-15RA), interleukin 10 (IL-10), programmed cell death protein 1 (PD-1), programmed death-ligand 1 (PD-L1), T cell immunoreceptor with Ig and ITIM domains (TIGIT), CD16A, CD2 and/or CD38.

The methods of generating genetically modified animal model with two or more human or chimeric genes (e.g., humanized genes) can include the following steps:
(a) using the methods of introducing human NKP46 gene or chimeric NKP46 gene as described herein to obtain a genetically modified non-human animal;
(b) mating the genetically modified non-human animal with another genetically modified non-human animal, and then screening the progeny to obtain a genetically modified non-human animal with two or more human or chimeric genes.

In some embodiments, in step (b) of the method, the genetically modified animal can be mated with a genetically modified non-human animal with human or chimeric NKG2D, EGFR, HER2, B7H3, BCMA, FAP, CXCR4, CSF2, TNFR2, IL-2, IL-15, IL-15RA, IL-10, PD-1, PD-L1, TIGIT, CD16A, CD2 and/or CD38 gene. Some of these genetically modified non-human animal are described, e.g., in PCT/CN2017/090320, PCT/CN2017/099574, PCT/CN2017/099576, PCT/CN2022/113594, PCT/CN2021/095273, PCT/CN2022/096667, PCT/CN2020/113618, PCT/CN2019/128358, PCT/CN2020/128201, PCT/CN2022/131092, and PCT/CN2021/085053; each of which is incorporated herein by reference in its entirety.

In some embodiments, the NKP46 humanization is directly performed on a genetically modified animal having a human or chimeric NKG2D, EGFR, HER2, B7H3, BCMA, FAP, CXCR4, CSF2, TNFR2, IL-2, IL-15, IL-15RA, IL-10, PD-1, PD-L1, TIGIT, CD16A, CD2 and/or CD38 gene.

As these proteins may involve different mechanisms, a combination therapy that targets two or more of these proteins thereof may be a more effective treatment. In fact, many related clinical trials are in progress and have shown a good effect. The genetically modified animal model with two or more human or humanized genes can be used for determining effectiveness of a combination therapy that targets two or more of these proteins, e.g., an anti-NKP46 antibody and an additional therapeutic agent for the treatment of cancer. The methods include administering the anti-NKP46 antibody and the additional therapeutic agent to the animal, wherein the animal has a tumor; and determining the inhibitory effects of the combined treatment to the tumor. In some embodiments, the additional therapeutic agent is an antibody that specifically binds to NKG2D, EGFR, HER2, B7H3, BCMA, FAP, CXCR4, CSF2, TNFR2, IL-2, IL-15, IL-15RA, IL-10, PD-1, PD-L1, TIGIT, CD16A, CD2 and/or CD38. In some embodiments, the additional therapeutic agent is an anti-CTLA4 antibody (e.g., ipilimumab), an anti-PD-1 antibody (e.g., nivolumab), or an anti-PD-L1 antibody.

In some embodiments, the animal further comprises a sequence encoding a human or humanized PD-1, a sequence encoding a human or humanized PD-L1, or a sequence encoding a human or humanized CTLA-4. In some embodiments, the additional therapeutic agent is an anti-PD-1 antibody (e.g., nivolumab, pembrolizumab), an anti-PD-L1 antibody, or an anti-CTLA-4 antibody. In some embodiments, the tumor comprises one or more tumor cells that express CD80, CD86, PD-L1, and/or PD-L2.

In some embodiments, the combination treatment is designed for treating various cancers as described herein, e.g., a solid tumor, gynecologic cancer, breast cancer, colorectal cancer, gastric adenocarcinoma, lung adenocarcinoma, pancreatic cancer, or head and neck cancer.

In some embodiments, the methods described herein can be used to evaluate the combination treatment with some other methods. The methods of treating a cancer that can be used alone or in combination with methods described herein, include, e.g., treating the subject with chemotherapy, e.g., camptothecin, doxorubicin, cisplatin, carboplatin, procarbazine, mechlorethamine, cyclophosphamide, adriamycin, ifosfamide, melphalan, chlorambucil, bisulfan, nitrosurea, dactinomycin, daunorubicin, bleomycin, plicomycin, mitomycin, etoposide, verpamil, podophyllotoxin, tamoxifen, taxol, transplatinum, 5-flurouracil, vincristine, vinblastin, and/or methotrexate. Alternatively or in addition, the methods can include performing surgery on the subject to remove at least a portion of the cancer, e.g., to remove a portion of or all of a tumor(s), from the patient.

### EXAMPLES

The invention is further described in the following examples, which do not limit the scope of the invention described in the claims.

### Materials and Methods

The following materials were used in the following examples.

C57BL/6 mice and Flp transgenic mice were purchased from the China Food and Drugs Research Institute National Rodent Experimental Animal Center.

BALB/c mice were purchased from Beijing Vital River Laboratory Animal Technology Co., Ltd.

BspHI, Stul, BbsI, EcoRI, and BamHI restriction enzymes were purchased from NEB (Catalog numbers: R0517S, R0187S, R0539L, R0101M, and R0136M, respectively).

Brilliant Violet 510™ anti-mouse CD45 Antibody was purchased from BioLegend (Catalog number: 103138).

PerCP/Cyanine5.5 anti-mouse TCRB chain Antibody was purchased from BioLegend (Catalog number: 109228).

PE/Cy™ 7 Mouse anti-mouse NK1.1 was purchased from BioLegend (Catalog number: 552878).

PE anti-human CD335 (NKP46) Antibody was purchased from BioLegend (Catalog number: 331907).

APC anti-mouse CD335 (NKP46) Antibody was purchased from BioLegend (Catalog number: 137607).

Zombie NIR™ Fixable Viability Kit was purchased from BioLegend (Catalog number: 423106).

Purified anti-mouse CD16/32 Antibody was purchased from BioLegend (Catalog number: 101302).

PrimeScript™ RT reagent Kit with gDNA Eraser was purchased from Takara Bio (Catalog number: 6110A).

APC Rat IgG2a, λ Isotype Ctrl Antibody was purchased from BioLegend (Catalog number: 402306).

PE Mouse IgG1, K Isotype Ctrl Antibody was purchased from BioLegend (Catalog number: 400112).

PerCP anti-mouse Ly-6G/Ly-6C (Gr-1) Antibody was purchased from BioLegend (Catalog number: 108426).

Brilliant Violet 421™ anti-mouse CD4 Antibody was purchased from BioLegend (Catalog number: 100438).

FITC anti-mouse F4/80 Antibody was purchased from BioLegend (Catalog number: 123108).

PE anti-mouse CD8a Antibody was purchased from BioLegend (Catalog number: 100708).

Brilliant Violet 605™ anti-mouse CD19 Antibody was purchased from BioLegend (Catalog number: 115540).

Brilliant Violet 605™ anti-mouse CD11c Antibody was purchased from BioLegend (Catalog number: 117334).

PE anti-mouse/human CD11b Antibody was purchased from BioLegend (Catalog number: 101208).

FITC Rat Anti-Mouse CD3 Molecular Complex was purchased from BD Pharmingen (Catalog number: 561798).

APC Hamster Anti-Mouse TCR β Chain was purchased from BD Pharmingen (Catalog number: 553174).

APC anti-mouse/rat Foxp3 was purchased from eBioscience (Catalog number: 17-5773-82).

### Example 1: Mice with Humanized NKP46 Gene

In this example, a non-human animal (e.g., a mouse) was modified to include a nucleotide sequence encoding human NKP46 protein, and the obtained genetically-modified non-human animal can express a human or humanized NKP46 protein in vivo. The mouse NKP46 gene (NCBI Gene ID: 17086, Primary source: MGI: 1336212, UniProt ID: Q8C567) is located at 4340714 to 4348183 of chromosome 7 (NC_000073.7), and the human NKP46 gene (NCBI Gene ID: 9437, Primary source: HGNC: 6731, UniProt ID: 076036) is located at 54906063 to 54938211 of chromosome 19 (NC_000019.10). The mouse NKP46 transcript is NM_010746.3, and the corresponding protein sequence NP_034876.2 is set forth in SEQ ID NO: 1. The human NKP46 transcript is NM_004829.7, and the corresponding protein sequence NP 004820.2 is set forth in SEQ ID NO: 2. Mouse and human NKP46 gene loci are shown in FIG. 1.

All or part of nucleotide sequences encoding human NKP46 protein can be introduced into the mouse endogenous NKP46 locus, so that the mouse expresses human or humanized NKP46 protein. Specifically, using gene-editing techniques, under control of mouse NKP46 gene regulatory elements, a sequence (about 6.7 kb) starting from within exon 1 and ending within exon 7 of mouse NKP46 gene was replaced with a corresponding sequence (about 6.5 kb) starting from within exon 1 and ending within exon 7 of human NKP46 gene, to obtain a humanized NKP46 gene locus as shown in FIG. 2, thereby humanizing mouse NKP46 gene.

As shown in the schematic diagram of the targeting strategy in FIG. 3, the targeting vector contains homologous arm sequences upstream and downstream of the mouse NKP46 gene, and an “A Fragment” containing DNA sequences of human NKP46 gene. Specifically, sequence of the upstream homologous arm (5′ homologous arm, SEQ ID NO: 3) is identical to nucleotide sequence of 4336716-4340750 of NCBI accession number NC_000073.7, and sequence of the downstream homologous arm (3′ homologous arm, SEQ ID NO: 4) is identical to nucleotide sequence of 4348522-4352569 of NCBI accession number NC_000073.7. The genomic DNA sequence from human NKP46 gene (SEQ ID NO: 7) is 99% identical to nucleotide sequence of 54906191-54912715 of NCBI accession number NC_000019.10 (position 54906696 was mutated from “C” to “A”).

The targeting vector also includes an antibiotic resistance gene for positive clone screening (neomycin phosphotransferase gene, or Neo), and two Frt recombination sites flanking the anti biotic resistance gene, that formed a Neo cassette. The connection between the 5′ end of the Neo cassette and the mouse sequence was designed as:

(SEQ ID NO: 10)
5′-CTCCTCCACCCTTCACTTTCTTCTCTTCAAACACTCCCGGTACCCT
CGAGGTCGACGGTATCGATAAGCTTGATATCGAATTCCGAAGTTCCTAT
TC-3′,

wherein the last “C” in sequence “TCCC” is the las t nucleotide of the mouse sequence, and the first “G” in sequence “GGTA” is the first nucleotide of the Neo cassette. The connection between the 3′ end of the Neo cassette and the mouse sequence was designed as:

(SEQ ID NO: 11)
5′-TATAGGAACTTCATCAGTCAGGTACATAATGGTGGATCCATATACC
CCTCTTACTCTCTTTCAAATTCATGGCCTCTAGTT-3′,

wherein the last “C” in sequence “ATCC” is the last nucleotide of the Neo cassette, and the first “A” in sequence “ATAT” is the first nucleotide of the mouse sequence. In addition, a coding gene with a negative selectable marker (a gene encoding diphtheria toxin A subunit (DTA)) was also constructed downstream of the 3′ homologous arm of the targeting vector. The mRNA sequence of the engineered mouse NKP46 after humanization and its encoded protein sequence are shown in SEQ ID NO: 8 and SEQ ID NO: 9, respectively.

Given that human NKP46 has multiple isoforms or transcripts, the methods described herein can be applied to other isoforms or transcripts.

The targeting vector was constructed, e.g., by restriction enzyme digestion and ligation. The constructed targeting vector sequences were preliminarily confirmed by restriction enzyme digestion, and then verified by sequencing. Embryonic stem cells of C57BL/6 mice were transfected with the correct targeting vector by electroporation. The positive selectable marker genes were used to screen the cells, and the integration of exogenous genes was confirmed by PCR and Southern Blot. The clones identified as positive by PCR (primers shown in the table below) were then verified by Southern Blot. The clones that were positive in Southern Blot detection were further sequenced. Clones verified to have no random insertion were subjected for subsequent experiments.

TABLE 3
PCR primer sequences and target fragment sizes

| | | Target |
|---|---|---|
| Primer | Primer sequence (5′-3′) | fragment size |
| L-GT-F | ATCACCATGCCTGACGTTAAGAAGGA | Mut: 5423 bp |
| | (SEQ ID NO: 12) | |
| L-GT-R | TTCTTCCTCACATGACCAACCAGGC | |
| | (SEQ ID NO: 13) | |

The positive clones that had been screened (black mice) were introduced into isolated blastocysts (white mice), and the resulted chimeric blastocysts were transferred to a culture medium for short-term culture and then transplanted to the fallopian tubes of the recipient mother (white mice) to produce the F0 chimeric mice (black and white). The F2 generation homozygous mice were obtained by backcrossing the F0 generation chimeric mice with wild-type mice to obtain the F1 generation mice, and then breeding the F1 generation heterozygous mice with each other. The positive mice were also bred with the Flp transgenic mice to remove the positive selectable marker genes (schematic diagram shown in FIG. 4), and then the humanized homozygous mice with a humanized NKP46 gene were obtained by breeding the heterozygous mice with each other.

In addition, the CRISPR/Cas system can also be used for gene editing, and the targeting strategy shown in FIG. 5 was designed. The targeting vector contains homologous arm sequences upstream and downstream of the mouse NKP46 gene, and a human NKP46 gene fragment. Specifically, sequence of the upstream homologous arm (5′ homologous arm, SEQ ID NO: 5) is identical to nucleotide sequence of 4339331-4340750 of NCBI accession number NC_000073.7, and sequence of the downstream homologous arm (3′ homologous arm, SEQ ID NO: 6) is 99% identical to nucleotide sequence of 4347485-4348999 of NCBI accession number NC 000073.7. The human NKP46 gene fragment (SEQ ID NO: 7) is 99% identical to nucleotide sequence of 54906191-54912715 of NCBI accession number NC_000019.10 (position 54906696 was mutated from “C” to “A”). The mRNA sequence of the engineered mouse NKP46 after humanization and its encoded protein sequence are shown in SEQ ID NO: 8 and SEQ ID NO: 9, respectively.

The targeting vector was constructed, e.g., by restriction enzyme digestion, ligation, or direct synthesis. The constructed targeting vector sequences were preliminarily confirmed by restriction enzyme digestion, and then verified by sequencing. Targeting vectors with verified sequences were used for subsequent experiments.

Specific sgRNA sequences were designed and synthesized that recognize the targeting site. The targeting site sequences of the sgRNAs on the NKP46 gene locus are as follows:

sgRNA1 targeting site (SEQ ID NO: 14):
5′-TTGAATCAAGAGCAGATTGGGGG-3′;
sgRNA2 targeting site (SEQ ID NO: 15):
5′-GAATCTCATTCGAATTGGTCTGG-3′.

UCA kit was used to detect the activity of the sgRNAs. After confirming that the sgRNAs can induce efficient Cas9 cleavage, restriction enzyme cleavage sites were added to its 5′ end and a complementary strand to obtain a forward oligonucleotide and a reverse oligonucleotide, as shown in the table below. After annealing, the products were ligated to the pT7-sgRNA plasmid (the plasmid was first linearized with BbsI), to obtain expression vector pT7-NKP46-1 and pT7-NKP46-2.

TABLE 4
sgRNA1 and sgRNA2 sequence list
sgRNA1 sequence

| | |
|---|---|
| SEQ ID NO: 16 | Upstream: 5′-TTGAATCAAGAGCAGATTGG-3′ |
| SEQ ID NO: 17 | Upstream: 5′-TAGGTTGAATCAAGAGCAGATTGG-3′ |
| (forward oligonucleotide) | |
| SEQ ID NO: 18 | Downstream: 5′-CCAATCTGCTCTTGATTCAA-3′ |
| SEQ ID NO: 19 | Downstream: 5′-AAACCCAATCTGCTCTTGATTCAA-3′ |
| (reverse oligonucleotide) | |

sgRNA2 sequence

| | |
|---|---|
| SEQ ID NO: 20 | Upstream: 5′-GAATCTCATTCGAATTGGTC-3′ |
| SEQ ID NO: 21 | Upstream: 5′-TAGGGAATCTCATTCGAATTGGTC-3′ |
| (forward oligonucleotide) | |
| SEQ ID NO: 22 | Downstream: 5′-GACCAATTCGAATGAGATTC-3′ |
| SEQ ID NO: 23 | Downstream: 5′-AAACGACCAATTCGAATGAGATTC-3′ |
| (reverse oligonucleotide) | |

The pT7-sgRNA vector was synthesized, which included a DNA fragment containing the T7 promoter and sgRNA scaffold (SEQ ID NO: 24), and was ligated to the backbone vector (Takara, Catalog number: 3299) after restriction enzyme digestion (EcoRI and BamHI). The resulting plasmid was confirmed by sequencing.

The pre-mixed Cas9 mRNA, the targeting vector, and in vitro transcription products of the pT7-NKP46-1 and pT7-NKP46-2 plasmids (using Ambion™ in vitro transcription kit to carry out the transcription according to the method provided in the product instruction) were injected into the cytoplasm or nucleus of fertilized eggs of C57BL/6 or BALB/c mice with a microinjection instrument. The embryo microinjection was carried out according to the method described, e.g., in A. Nagy, et al., “Manipulating the Mouse Embryo: A Laboratory Manual (Third Edition),” Cold Spring Harbor Laboratory Press, 2006. The injected fertilized eggs were then transferred to a culture medium to culture for a short time and then was transplanted into the oviduct of the recipient mouse to produce the genetically modified mice (F0 generation). The mouse population was further expanded by cross-breeding and self-breeding to establish stable homozygous mouse lines.

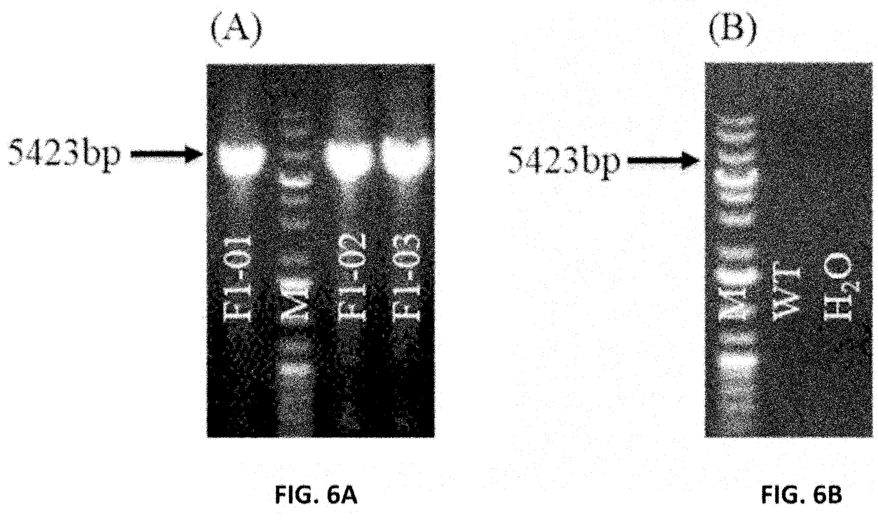

The genotype of the somatic cells of the F1 generation mice can be identified by PCR analysis. The PCR primers are shown in Table 3. The identification results of some F1 generation mice are shown in FIGS. 6A-6B. The results indicate that mice numbered F1-01, F1-02, and F1-03 were positive heterozygous mice.

The F1 generation mice identified as positive by PCR were further verified by Southern Blot to confirm whether there was random insertion. Specifically, genomic DNA from the mouse tail was extracted, which was digested with BspHI or Stul restriction enzyme. The digested genomic DNA was then transferred to a membrane and hybridized with respective probes. The restriction enzymes, probes, and the size of target fragment are shown in the table below.

TABLE 5
Enzymes and probes used in Southern Blot

| Restriction | | Wild-type | Recombinant |
|---|---|---|---|
| enzyme | Probe | fragment size | fragment size |
| BspHI | A Probe | — | 8.6 kb |
| StuI | 3′Probe | 18.1 kb | 14.6 kb |

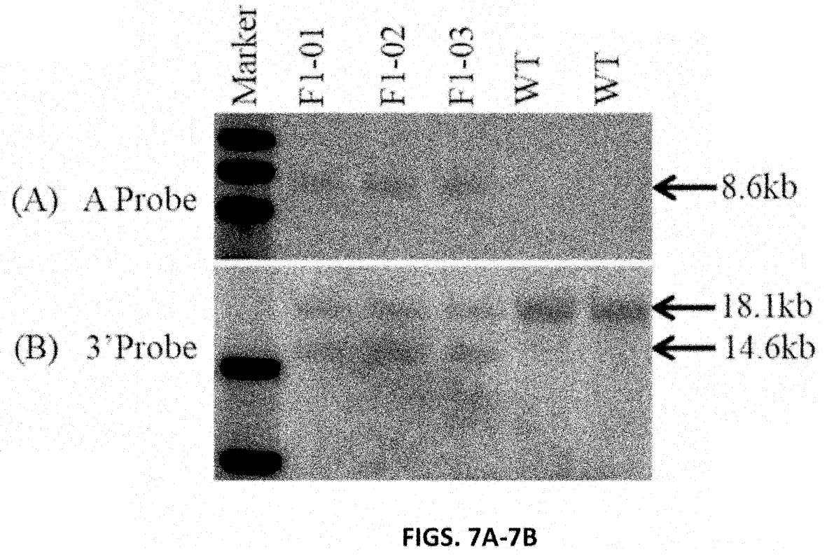

The Southern Blot detection results are shown in FIGS. 7A-7B. The results showed that mice numbered F1-01, F1-02, and F1-03 were verified as positive heterozygous mice. The results indicate that the NKP46 gene humanized mice constructed using the methods described herein can be stably passaged without random insertions.

The following primers were used for probe synthesis in Southern Blot assays:

A Probe-F (SEQ ID NO: 25):
5′-GCAGTGTTTGTGTCCCTGGGTACTT-3′,
A Probe-R (SEQ ID NO: 26):
5′-TACCCAACAGCTCATTGAGAACGGG-3′;
3′Probe-F (SEQ ID NO: 27):
5′-TCTACCGCCATGACCATAGCACCTA-3′,
3′Probe-R (SEQ ID NO: 28):
5′-TCTGCTCTCTTCCATGTTGGTTCCT-3′.

The expression of human or humanized NKP46 protein in positive mice can also be confirmed, e.g., by flow cytometry. Specifically, one 6-week-old female C57BL/6 wild-type mouse and one 6-week-old female NKP46 gene humanized heterozygous mouse were selected. Peripheral blood and spleen tissues were collected after euthanasia by cervical dislocation. Cells were stained with: Brilliant Violet 510™ anti-mouse CD45 (mCD45; an anti-mouse CD45 antibody), PerCP/Cyanine5.5 anti-mouse TCRB chain Antibody (mTCRβ; an anti-mouse TCRB antibody), PE/Cy™ 7 Mouse anti-mouse NK1.1 (mNK1.1; an anti-mouse NK1.1 antibody), PE anti-human CD335 (NKP46) Antibody (hNKP46; an anti-human NKP46 antibody), APC anti-mouse CD335 (NKP46) Antibody (mNKP46; an anti-mouse NKP46 antibody), Zombie NIR™ Fixable Viability Kit, and/or Purified anti-mouse CD16/32 Antibody (an anti-mouse CD16/32 antibody), and then subjected to flow cytometry analysis. The results are shown in the table below.

TABLE 6
NKP46 protein detection results by flow cytometry

| | NKP46 positive | | hNKP46 |
|---|---|---|---|
| Cell type | cell characteristics | C57BL/6 | (heterozygous) |
| Spleen NK cells | mCD45 + mNK1.1 + hNKP46+ | 0.14% | 4.08% |
| | mCD45 + mNK1.1 + mNKP46+ | 4.21% | 3.44% |
| Peripheral blood | mCD45 + mNK1.1 + hNKP46+ | 0.056% | 4.95% |
| NK cells | mCD45 + mNK1.1 + mNKP46+ | 3.96% | 4.33% |

The above table shows that only mouse NKP46 protein, but not human or humanized NKP46 protein, was detected in the wild-type C57BL/6 mouse; whereas humanized NKP46 protein was only detected in the NKP46 gene humanized heterozygous mouse.

Similarly, the expression of humanized NKP46 protein in NKP46 gene humanized homozygous mice can also be confirmed, e.g., by flow cytometry. Specifically, one 5-week-old female C57BL/6 wild-type mouse and one 5-6 weeks old female NKP46 gene humanized homozygous mouse were selected. Peripheral blood and spleen tissues were collected after euthanasia by cervical dislocation. Cells were stained using the same antibodies/kits as described above, together with APC Rat IgG2a, λ Isotype Ctrl Antibody, and PE Mouse IgG1, κ Isotype Ctrl Antibody. The stained cells were then subject to flow cytometry analysis. The results are shown in the table below.

TABLE 7
NKP46 protein detection results by flow cytometry

| | NKP46 positive | | hNKP46 |
|---|---|---|---|
| Cell type | cell characteristics | C57BL/6 | (homozygous) |
| Spleen NK cells | mCD45 + mNK1.1 + hNKP46+ | 0.11% | 2.79% |
| | mCD45 + mNK1.1 + mNKP46+ | 2.25% | 0.035% |
| Peripheral blood | mCD45 + mNK1.1 + hNKP46+ | 0.034% | 3.55% |
| NK cells | mCD45 + mNK1.1 + mNKP46+ | 2.81% | 0.009% |

The above table shows that only mouse NKP46 protein, but not human or humanized NKP46 protein, was detected in the wild-type C57BL/6 mouse; whereas only humanized NKP46 protein, but not mouse NKP46 protein, was detected in the NKP46 gene humanized homozygous mouse. The results indicate that humanized NKP46 protein can be normally expressed in NKP46 gene humanized homozygous mice.

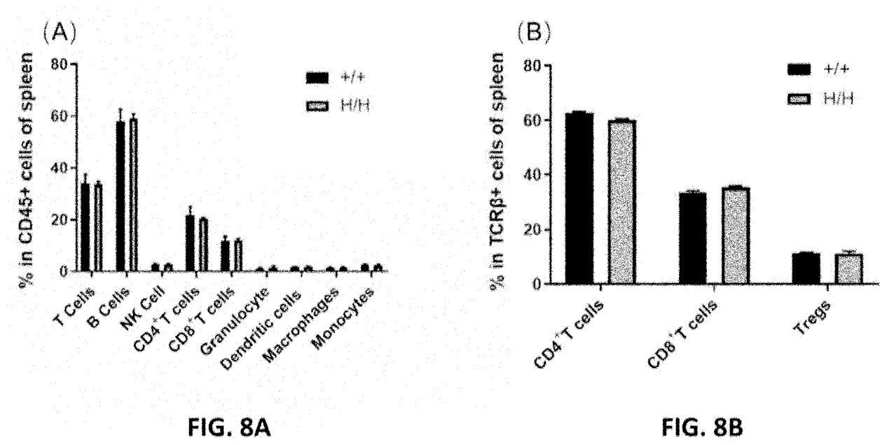

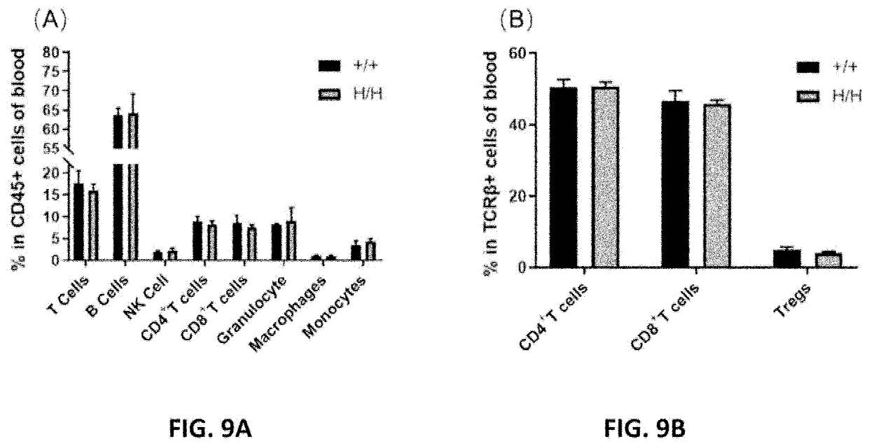

Further, the spleen, lymph nodes, and peripheral blood from C57BL/6 wild-type mice (+/+) and NKP46 gene humanized homozygous mice (H/H) were collected for immuno-phenotyping detection by flow cytometry. Specifically, three 6-week-old female C57BL/6 wild-type mice and three 6-week-old female NKP46 gene humanized homozygous mice were selected, and the spleen, lymph nodes, and peripheral blood were collected after euthanasia by cervical dislocation. Cells in these tissues were stained with: Purified anti-mouse CD16/32 Antibody, Zombie NIR™ Fixable Viability Kit, Brilliant Violet 510™ anti-mouse CD45 Antibody, PerCP anti-mouse Ly-6G/Ly-6C (Gr-1) Antibody, Brilliant Violet 421™ anti-mouse CD4 Antibody, FITC anti-mouse F4/80 Antibody, PE anti-mouse CD8a Antibody, PE/Cy™ 7 Mouse anti-mouse NK1.1, FITC Rat Anti-Mouse CD3 Molecular Complex, APC Hamster Anti-Mouse TCRB Chain, APC anti-mouse/rat Foxp3, Brilliant Violet 605™ anti-mouse CD19 Antibody, Brilliant Violet 605™ anti-mouse CD11c Antibody, and/or PE anti-mouse/human CD11b Antibody, and then subject to immuno-phenotyping detection. The detection results of leukocyte subtypes and T cell subtypes in the spleen and peripheral blood are shown in FIGS. 8A-8B and FIGS. 9A-9B, respectively. The results showed that the percentages of B cells, T cells, NK cells, CD4⁺ T cells, CD8⁺ T cells, granulocytes, macrophages, monocytes, and other leukocyte subtypes in the spleen and peripheral blood of NKP46 gene humanized homozygous mice were basically the same as those in C57BL/6 wild-type mice (FIG. 8A and FIG. 9A). In addition, the percentages of CD4⁺ T cells, CD8⁺ T cells, and Treg cells (Tregs) were basically the same as those in C57BL/6 wild-type mice (FIG. 8B and FIG. 9B).

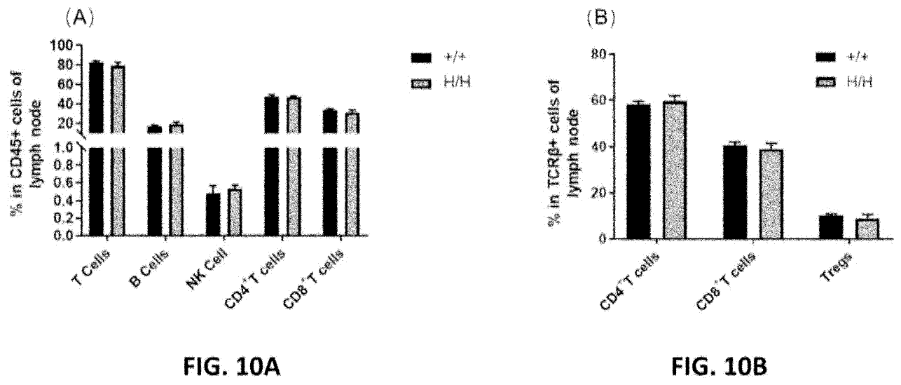

The detection results of leukocyte subtypes and T cell subtypes in lymph nodes are shown in FIG. 10A and FIG. 10B, respectively. The results showed that the leukocyte subtypes, e.g., T cells, B cells, NK cells, CD4⁺ T cells, CD8⁺ T cells, and other leukocyte subtypes in the lymph nodes of NKP46 gene humanized homozygous mice were basically the same as those of C57BL/6 wild-type mice (FIG. 10A). In addition, the percentages of T cell subtypes, e.g., CD4⁺ T cells, CD8⁺ T cells and Tregs cells were basically the same as those of C57BL/6 wild-type mice (FIG. 10B).

The results indicate that the humanization of NKP46 gene did not affect the differentiation, development and distribution of leukocytes and T cells in the spleen, lymph nodes and peripheral blood of mice.

### Example 2: In Vivo Efficacy Verification

The NKP46 gene humanized mice prepared by the methods described herein can be used to evaluate the efficacy of antibodies targeting human NKP46. For example, the NKP46 gene humanized homozygous mice can be subcutaneously inoculated with tumor cells (e.g., mouse colon cancer cells MC38). When the tumor volume reaches about 100 mm³, the mice can be placed into a control group and one or more treatment groups according to the tumor volume. The treatment group mice can be administered with randomly selected drugs targeting human NKP46 (e.g., anti-human NKP46 antibodies), and the control group mice can be injected with an equal volume of PBS. The tumor volume and body weight of the mice can be measured regularly, and the in vivo safety and efficacy of the drugs can be effectively assessed by comparing the changes in the body weight of the mice and the tumor size.

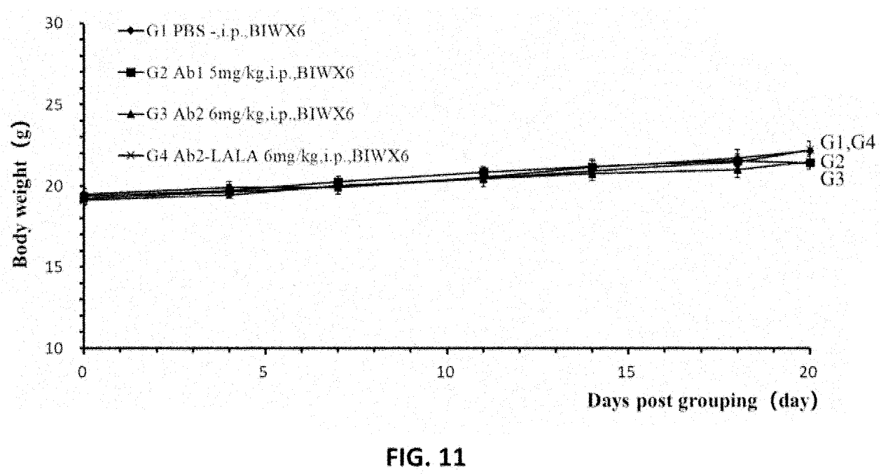

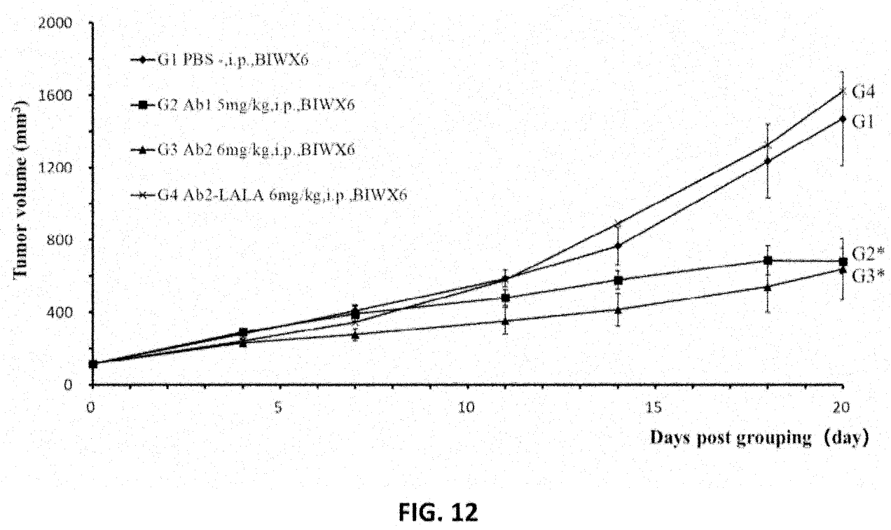

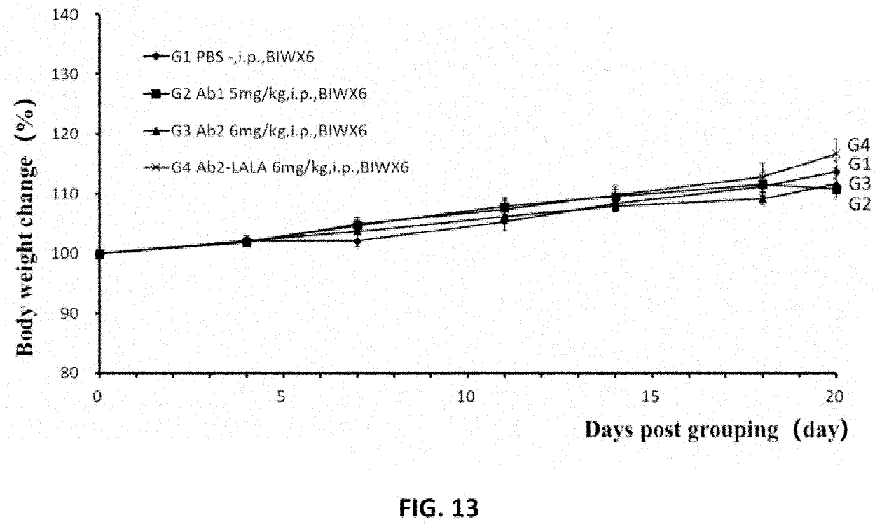

Specifically, 7-8 weeks old NKP46 gene humanized homozygous mice were subcutaneously inoculated with colon cancer cells MC38 (5×10⁵). After the tumor volume grew to about 100 mm³, the mice were placed into a control group and three treatment groups (7 mice per group). Mice in the treatment groups G2 and G3 were injected with IgG1 antibody drugs Ab1 and Ab2 targeting human NKP46, and the treatment group mice (G4) were injected with Ab2-LALA (Ab2 with LALA mutations). The control group mice (G1) were injected with an equal volume of PBS. All mice were administered twice a week (6 times in total). The tumor volume and body weight of the mice were measured twice a week until the end of the experiment on Day 20 (20 days after grouping). Euthanasia was performed when the tumor volume of a single mouse reached 3000 mm³ after inoculation. The specific grouping, administration, dosage and frequency are shown in the table below. The body weight, tumor volume, and body weight change of the mice during the experiment are shown in FIG. 11, FIG. 12, and FIG. 13, respectively.

TABLE 8
Specific grouping, administration, dosage, and frequency

| Group | Drug | Dosage | Frequency |
|---|---|---|---|
| G1 | PBS | — | Twice a week (BIW), 6 |
| | | | administrations in total |
| G2 | Ab1 | 5 mg/kg | Twice a week (BIW), 6 |
| | | | administrations in total |
| G3 | Ab2 | 6 mg/kg | Twice a week (BIW), 6 |
| | | | administrations in total |
| G4 | Ab2-LALA | 6 mg/kg | Twice a week (BIW), 6 |
| | | | administrations in total |

Overall, the animals in each group were in good health during the experiment. At the end of the experiment (20 days after grouping), the weight of mice in all treatment groups (G2, G3, and G4) and control group (G1) increased, and there was no significant difference in body weight and body weight change throughout the experimental period (FIG. 11 and FIG. 13). Based on the tumor volume measurement results (FIG. 12), the tumors of the control group mice continued to grow during the experimental period. However, the tumor volume growth of the treatment group mice (G2 and G3) showed different degrees of inhibition and/or shrinkage. The results indicate that anti-human NKP46 antibodies (G2 and G3) had different tumor-inhibiting effects in mice, while the subtype-modified antibody drug (G4) had no tumor-inhibiting effect.

TABLE 9
The results of tumor volume and survival of mice in each group

| | | | | Survived | Tumor-free | | P value | |
|---|---|---|---|---|---|---|---|---|
| | Tumor volume (mm³) | | | mice on | mice on | | Body | Tumor |
| Group | Day 0 | Day 11 | Day 20 | Day 20 | Day 20 | TGI_{TV} % | weight | volume |
| G1 | 117 ± 2 | 588 ± 45 | 1470 ± 260 | 7/7 | 0/7 | N/A | N/A | N/A |
| G2 | 117 ± 2 | 482 ± 45 | 685 ± 70 | 7/7 | 0/7 | 58.1 | 0.301 | 0.013 |
| G3 | 117 ± 3 | 354 ± 74 | 639 ± 170 | 7/7 | 0/7 | 61.4 | 0.361 | 0.020 |
| G4 | 117 ± 2 | 581 ± 80 | 1624 ± 369 | 7/7 | 0/7 | −11.3 | 0.938 | 0.740 |

The above table lists the main data and analysis results of each experiment, specifically including the tumor volume, the number of survived mice on Day 20, the number of tumor-free mice on Day 20, Tumor Growth Inhibition value based on tumor volume (TGI_{TV}), and the statistical difference (P value) of the mouse body weight and tumor volume between the treatment group and the control group, at the time of grouping (Day 0), 11 days after grouping (Day 11), and the end of the experiment (20 days after grouping or Day 20).

According to the above table, at the end of the experiment (Day 20), the average tumor volume of the control group mice (G1) was 1470=260 mm³, while the average tumor volumes of G2, G3, and G4 group mice were 685±70 mm³, 639±170 mm³, and 1624±369 mm³, respectively. The tumor volume of the mice in the G2 and G3 groups was significantly smaller than that of the control group (G1), and there was a significant difference compared with the tumor volume of the control group (p<0.05). The TGI_{TV} of the treatment groups G2 and G3 were 58.1% and 61.4%, respectively, indicating that Ab1 and Ab2 can treat tumors and inhibit tumor growth in NKP46 gene humanized homozygous mice, while the subtype-modified Ab2-LALA had no inhibitory effect on tumor.

In summary, the NKP46 gene humanized animal model can be used as an in vivo model for drug efficacy research, and used for the screening, evaluation and treatment experiments of NKP46 signaling pathway regulators. The animal model can also be used to evaluate the efficacy of antibodies targeting human NKP46 in vivo, and to evaluate the therapeutic effect of NKP46-targeting drugs.

### Example 3: Generation of Double- or Multi-Gene Humanized Mice

The NKP46 gene humanized mice generated using the methods described herein can also be used to generate double- or multi-gene humanized mouse models. For example, in Example 1, the embryonic stem (ES) cells for blastocyst microinjection can be selected from mice comprising other genetic modifications such as modified (e.g., human or humanized) NKG2D, EGFR, HER2, B7H3, BCMA, FAP, CXCR4, CSF2, TNFR2, IL-2, IL-15, IL-15RA, IL-10, PD-1, PD-L1, TIGIT, CD16A, CD2 and/or CD38 genes. Alternatively, embryonic stem cells from humanized NKP46 mice described herein can be isolated, and gene recombination targeting technology can be used to obtain double-gene or multi-gene-modified mouse models of NKP46 and other gene modifications. In addition, it is also possible to breed the homozygous or heterozygous NKP46 gene humanized mice obtained by the methods described herein with other genetically modified homozygous or heterozygous mice, and the offspring can be screened. According to Mendel's law, it is possible to generate double-gene or multi-gene modified heterozygous mice comprising modified (e.g., human or humanized) NKP46 gene and other genetic modifications. Then the heterozygous mice can be bred with each other to obtain homozygous double-gene or multi-gene modified mice. These double-gene or multi-gene modified mice can be used for in vivo validation of gene regulators targeting human NKP46 and other genes.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A genetically-modified, non-human animal whose genome comprises at least one chromosome comprising a sequence encoding a human or chimeric natural cytotoxicity triggering receptor 1 (NKP46).

2. The animal of claim 1, wherein the sequence encoding the human or chimeric NKP46 is operably linked to an endogenous regulatory element at the endogenous NKP46 gene locus in the at least one chromosome.

3. The animal of claim 1 or 2, wherein the sequence encoding a human or chimeric NKP46 comprises a sequence encoding an amino acid sequence that is at least 70%, 75%, 80%, 85%, 90%, 95%, 99%, or 100% identical to human NKP46 (NP_004820.2 (SEQ ID NO: 2)).

4. The animal of claim 1 or 2, wherein the sequence encoding a human or chimeric NKP46 comprises a sequence encoding an amino acid sequence that is at least 70%, 75%, 80%, 85%, 90%, 95%, 99%, or 100% identical to SEQ ID NO: 9.

5. The animal of claim 1 or 2, wherein the sequence encoding a human or chimeric NKP46 comprises a sequence encoding an amino acid sequence that is at least 70%, 75%, 80%, 85%, 90%, 95%, 99%, or 100% identical to amino acids 1-253 or 2-253 of SEQ ID NO: 2.

6. The animal of any one of claims 1-5, wherein the animal is a mammal, e.g., a monkey, a rodent, a mouse, or a rat.

7. The animal of any one of claims 1-6, wherein the animal is a mouse.

8. The animal of any one of claims 1-7, wherein the animal does not express endogenous NKP46 or expresses a decreased level of endogenous NKP46 as compared to NKP46 expression level in a wild-type animal.

9. The animal of any one of claims 1-8, wherein the animal has one or more cells expressing human or chimeric NKP46.

10. The animal of any one of claims 1-9, wherein the animal has one or more NK cells expressing human or chimeric NKP46, and the expressed human or chimeric NKP46 can induce the anti-tumor activity of the NK cells and/or bind to viral hemagglutinin.

11. A genetically-modified, non-human animal, wherein the genome of the animal comprises a replacement of a sequence encoding a region of endogenous NKP46 with a sequence encoding a corresponding region of human NKP46 at an endogenous NKP46 gene locus.

12. The animal of claim 11, wherein the sequence encoding the corresponding region of human NKP46 is operably linked to an endogenous regulatory element at the endogenous NKP46 locus, and one or more cells of the animal expresses a human or chimeric NKP46.

13. The animal of claim 11 or 12, wherein the animal does not express endogenous NKP46 or expresses a decreased level of endogenous NKP46 as compared to NKP46 expression level in a wild-type animal.

14. The animal of any one of claims 11-13, wherein the replaced sequence encodes all or a portion of the extracellular region of NKP46, optionally including the signal peptide.

15. The animal of any one of claims 11-14, wherein the animal has one or more cells expressing a chimeric NKP46 having a signal peptide, an extracellular region, a transmembrane region, and a cytoplasmic region, wherein the extracellular region comprises a sequence that is at least 50%, 60%, 70%, 80%, 90%, 95%, or 99% identical to the extracellular region of human NKP46 (NP_004820.2 (SEQ ID NO: 2)).

16. The animal of claim 15, wherein the extracellular region of the chimeric NKP46 has a sequence that has at least 10, 20, 30, 40, 50, 60, 70, 80, 90, 100, 110, 120, 130, 140, 150, 160, 170, 180, 190, 200, 210, 220, 230, 231, 232, 233, 234, 235, 236, or 237 contiguous amino acids that are identical to a contiguous sequence present in the extracellular region of human NKP46 (e.g., amino acids 22-253 or 22-258 of SEQ ID NO: 2).

17. The animal of claim 15 or 16, wherein the signal peptide of the chimeric NKP46 has a sequence that is at least 1, 2, 3, 4, 5, 6, 7, 8, 9, 10, 11, 12, 13, 14, 15, 16, 17, 18, 19, 20, or 21 contiguous amino acids that are identical to a contiguous sequence present in the signal peptide of human NKP46 (e.g., amino acids 1-21 or 2-21 of SEQ ID NO: 2).

18. The animal of any one of claims 11-17, wherein the sequence encoding a region of endogenous NKP46 comprises exon 1, exon 2, exon 3, exon 4, exon 5, exon 6, and/or exon 7, or a part thereof, of the endogenous NKP46 gene.

19. The animal of claim 18, wherein the animal is a mouse.

20. The animal of any one of claims 11-19, wherein the animal is heterozygous with respect to the replacement at the endogenous NKP46 gene locus.

21. The animal of any one of claims 11-19, wherein the animal is homozygous with respect to the replacement at the endogenous NKP46 gene locus.

22. A method for making a genetically-modified, non-human animal, comprising: replacing in at least one cell of the animal, at an endogenous NKP46 gene locus, a sequence encoding a region of endogenous NKP46 with a sequence encoding a corresponding region of human NKP46.

23. The method of claim 22, wherein the sequence encoding the corresponding region of human NKP46 comprises exon 1, exon 2, exon 3, exon 4, exon 5, exon 6, and/or exon 7, or a part thereof, of a human NKP46 gene.

24. The method of claim 22 or 23, wherein the sequence encoding the corresponding region of human NKP46 comprises a portion of exon 1, exon 2, exon 3, exon 4, exon 5, exon 6, and a portion of exon 7, of a human NKP46 gene.

25. The method of any one of claims 22-24, wherein the sequence encoding the corresponding region of human NKP46 encodes amino acids 1-253 or 2-253 of SEQ ID NO: 2.

26. The method of any one of claims 22-25, wherein the region comprises all or a portion of the extracellular region, optionally the signal peptide, of NKP46.

27. The method of any one of claims 22-26, wherein the sequence encoding a region of endogenous NKP46 comprises exon 1, exon 2, exon 3, exon 4, exon 5, exon 6, and/or exon 7, or a part thereof, of the endogenous NKP46 gene.

28. The method of any one of claims 22-27, wherein the animal is a mouse, and the sequence encoding a region of endogenous NKP46 comprises a portion of exon 1, exon 2, exon 3, exon 4, exon 5, exon 6, and a portion of exon 7 of the endogenous NKP46 gene.

29. A non-human animal comprising at least one cell comprising a nucleotide sequence encoding a humanized NKP46 polypeptide, wherein the humanized NKP46 polypeptide comprises at least 50 contiguous amino acid residues that are identical to the corresponding contiguous amino acid sequence of a human NKP46, wherein the animal expresses the humanized NKP46 polypeptide.

30. The animal of claim 29, wherein the humanized NKP46 polypeptide has at least 50, 60, 70, 80, 90, 100, 110, 120, 130, 140, 150, 160, 170, 180, 190, 200, 210, 220, 230, 231, 232, 233, 234, 235, 236, or 237 contiguous amino acid residues that are identical to the corresponding contiguous amino acid sequence of human NKP46 extracellular region (e.g., amino acids 22-253 or 22-258 of SEQ ID NO: 2).

31. The animal of claim 29 or 30, wherein the humanized NKP46 polypeptide has at least 1, 2, 3, 4, 5, 6, 7, 8, 9, 10, 11, 12, 13, 14, 15, 16, 17, 18, 19, 20, or 21 contiguous amino acid residues that are identical to the corresponding contiguous amino acid sequence of human NKP46 signal peptide (e.g., amino acids 1-21 or 2-21 of SEQ ID NO: 2).

32. The animal of any one of claims 29-31, wherein the humanized NKP46 polypeptide comprises a sequence that is at least 90%, 95%, or 99% identical to amino acids 1-253 or 2-253 of SEQ ID NO: 2.

33. The animal of any one of claims 29-32, wherein the nucleotide sequence is operably linked to an endogenous NKP46 regulatory element of the animal.

34. The animal of any one of claims 29-33, wherein the chimeric NKP46 polypeptide comprises an endogenous NKP46 transmembrane region and/or an endogenous NKP46 cytoplasmic region.

35. The animal of any one of claims 29-34, wherein the nucleotide sequence is integrated to an endogenous NKP46 gene locus of the animal.

36. The animal of any one of claims 29-35, wherein the humanized NKP46 polypeptide has at least one mouse NKP46 activity and/or at least one human NKP46 activity.

37. A method of making a genetically-modified animal cell that expresses a chimeric NKP46, the method comprising:
replacing at an endogenous NKP46 gene locus, a nucleotide sequence encoding a region of endogenous NKP46 with a nucleotide sequence encoding a corresponding region of human NKP46, thereby generating a genetically-modified animal cell that includes a nucleotide sequence that encodes the chimeric NKP46, wherein the animal cell expresses the chimeric NKP46.

38. The method of claim 37, wherein the animal is a mouse.

39. The method of claim 37 or 38, wherein the chimeric NKP46 comprises a human or humanized NKP46 extracellular region; and a transmembrane and/or a cytoplasmic region of mouse NKP46.

40. The method of claim 39, wherein the chimeric NKP46 further comprises a human or humanized NKP46 signal peptide.

41. The method of any one of 37-40, wherein the nucleotide sequence encoding the chimeric NKP46 is operably linked to an endogenous NKP46 regulatory region, e.g., promoter.

42. The animal of any one of claims 1-21 and 29-36, wherein the animal further comprises a sequence encoding an additional human or chimeric protein.

43. The animal of claim 42, wherein the additional human or chimeric protein is natural killer group 2D (NKG2D), epidermal growth factor receptor (EGFR), receptor tyrosine-protein kinase erbB-2 (HER2), cluster of differentiation 276 (B7H3), B-cell maturation antigen (BCMA), fibroblast activation protein alpha (FAP), C—X—C chemokine receptor type 4 (CXCR4), colony stimulating factor 2 (CSF2), tumor necrosis factor receptor 2 (TNFR2), interleukin 2 (IL-2), interleukin 15 (IL-15), interleukin 15 receptor, alpha subunit (IL-15RA), interleukin 10 (IL-10), programmed cell death protein 1 (PD-1), programmed death-ligand 1 (PD-L1), T cell immunoreceptor with Ig and ITIM domains (TIGIT), CD16A, CD2 and/or CD38.

44. The method of any one of claims 22-28 and 37-41, wherein the animal or mouse further comprises a sequence encoding an additional human or chimeric protein.

45. The method of claim 44, wherein the additional human or chimeric protein is NKG2D, EGFR, HER2, B7H3, BCMA, FAP, CXCR4, CSF2, TNFR2, IL-2, IL-15, IL-15RA, IL-10, PD-1, PD-L1, TIGIT, CD16A, CD2 and/or CD38.

46. A method of determining effectiveness of a therapeutic agent for the treatment of cancer, comprising:
a) administering the therapeutic agent to the animal of any one of claims 1-21, 29-36, 42, and 43, wherein the animal has a tumor; and
b) determining inhibitory effects of the therapeutic agent to the tumor.

47. The method of claim 46, wherein the therapeutic agent is an anti-NKP46 antibody (e.g., an anti-human NKP46 antibody).

48. The method of claim 46 or 47, wherein the tumor comprises one or more cancer cells that are injected into the animal.

49. The method of any one of claims 46-48, wherein determining inhibitory effects of the anti-NKP46 antibody to the tumor involves measuring the tumor volume in the animal.

50. The method of any one of claims 46-49, wherein the cancer is lung cancer, leukemia, colon cancer, head and neck cancer, kidney cancer, pancreatic cancer, or gastric cancer.

51. A method of determining effectiveness of an anti-NKP46 antibody and an additional therapeutic agent for the treatment of cancer, comprising
a) administering the anti-NKP46 antibody and the additional therapeutic agent to the animal of any one of claims 1-21, 29-36, 42, and 43, wherein the animal has a tumor; and
b) determining inhibitory effects on the tumor.

52. The method of claim 51, wherein the animal further comprises a sequence encoding a human or chimeric PD-1, a human or chimeric PD-L1, and/or a human or chimeric CTLA4.

53. The method of claim 51 or 52, wherein the additional therapeutic agent is an anti-PD-1 antibody, an anti-PD-L1 antibody, or an anti-CTLA4 antibody.

54. The method of any one of claims 51-53, wherein the tumor comprises one or more tumor cells that express PD-L1.

55. The method of any one of claims 51-54, wherein the tumor comprises one or more cancer cells that are injected into the animal.

56. The method of any one of claims 51-55, wherein determining inhibitory effects of the treatment involves measuring the tumor volume in the animal.

57. The method of any one of claims 51-56, wherein the animal has lung cancer, leukemia, colon cancer, head and neck cancer, kidney cancer, pancreatic cancer, or gastric cancer.

58. A method of determining effectiveness of a therapeutic agent for treatment an immune disorder (e.g., an autoimmune disease), comprising:
a) administering the therapeutic agent to the animal of any one of claims 1-21, 29-36, 42, and 43, wherein the animal has the immune disorder; and
b) determining effects of the therapeutic agent to the immune disorder.

59. A method of determining effectiveness of a therapeutic agent for reducing an inflammation, comprising:
a) administering the therapeutic agent to the animal of any one of claims 1-21, 29-36, 42, and 43, wherein the animal has the inflammation; and
b) determining effects of the therapeutic agent to the inflammation.

60. The method of claim 59, wherein the inflammation is caused by a virus (e.g., influenza).

61. A method of determining toxicity of a therapeutic agent comprising:
a) administering the therapeutic agent to the animal of any one of claims 1-21, 29-36, 42, and 43; and
b) determining effects of the therapeutic agent to the animal.

62. The method of claim 61, wherein the therapeutic agent is an anti-NKP46 antibody.

63. The method of claim 61 or 62, wherein determining effects of the therapeutic agent to the animal involves measuring the body weight, red blood cell count, hematocrit, and/or hemoglobin of the animal.

64. A protein comprising an amino acid sequence, wherein the amino acid sequence is one of the following:
(a) an amino acid sequence set forth in SEQ ID NO: 1, 2, or 9;
(b) an amino acid sequence that is at least 90% identical to SEQ ID NO: 1, 2, or 9;
(c) an amino acid sequence that is at least 91%, 92%, 93%, 94%, 95%, 96%, 97%, 98%, or 99% identical to SEQ ID NO: 1, 2, or 9;
(d) an amino acid sequence that is different from the amino acid sequence set forth in SEQ ID NO: 1, 2, or 9 by no more than 10, 9, 8, 7, 6, 5, 4, 3, 2 or 1 amino acid; and
(e) an amino acid sequence that comprises a substitution, a deletion and/or insertion of one, two, three, four, five or more amino acids to the amino acid sequence set forth in SEQ ID NO: 1, 2, or 9.

65. A nucleic acid comprising a nucleotide sequence, wherein the nucleotide sequence is one of the following:
(a) a sequence that encodes the protein of claim 64;
(b) SEQ ID NO: 3, 4, 5, 6, 7, 8, 10, or 11;
(c) a sequence that is at least 90% identical to SEQ ID NO: 3, 4, 5, 6, 7, 8, 10, or 11; and
(d) a sequence that is at least 91%, 92%, 93%, 94%, 95%, 96%, 97%, 98%, or 99% identical to SEQ ID NO: 3, 4, 5, 6, 7, 8, 10, or 11.

66. A cell comprising the protein of claim 64 and/or the nucleic acid of claim 65.

67. An animal comprising the protein of claim 64 and/or the nucleic acid of claim 65.


SEQUENCE LISTING (29 sequences)

SEQ ID NO: 1 - protein, 325 aa, Mus musculus
MLPTLTALLCLGLCLSQRINTEKETLPKPIIWAKPSIMVTNGNSVNIWCQGAQSASEYQLYFEGSFFALERPKPSRSMNKVRFFISQMTSHTAGIYTCFYQSGELWSKSSNPLKLVVTGLYDTPNLWVYPQPEVTLGENVTFFCQLKTATSKFFLLKERGSNHIQNKYGNIQAEFPMGPVTRAHRGTYRCFGSYNDYAWSFPSEPVTLLITGGVENSSLAPTDPTSSLDYWEFDLSTNESGLQKDSAFWDHTTQNLIRIGLACIILITLVWLLTEDWLSKRKDHEEANRLTNWECRRRWRMQHYFEEEQRNAISMMELKATPGAL

SEQ ID NO: 2 - protein, 304 aa, Homo sapiens
MSSTLPALLCVGLCLSQRISAQQQTLPKPFIWAEPHFMVPKEKQVTICCQGNYGAVEYQLHFEGSLFAVDRPKPPERINKVQFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLRMGLAFLVLVALVWFLVEDWLSRKRTRERASRASTWEGRRRLNTQTL

SEQ ID NO: 3 - DNA, 4035 bp, synthetic construct
  source 1..4035: mol_type = other DNA
ggcatagtgacagacaagtagaccaatggaatagatttgaagacccagaaatgaacctacacacctatggtcacttgatcttcgacaaggaagctaaaaccatccagtggaagaaagacagcattttcatcaaatggtgctggcacaactggcagttatcatgtagaagaatgtgattcgatccattcctatctccttgtagtaaggtcaaatctaagtggatcaaggaactccacataaaaccagagacactgaaacttatagaggagaaagtggggaaaagcctcgaagatatgggcacagggaaaaaattcctgaacagaacagcaatggcttgtgctgtaagattgagaattgacaaatgggacctcatgaaactgcaaagcttctgcaaggcaaaagacaccttcagtaagacaaaaaggctaccaacatattgggaaaggatctttacctatcctaaatcagataggggactaatatccaatatataaaaagagctcaagaaggtggactccagaaaatcaaataaccccattaaaaaatggggctcagagctaaacaaagaattctcacctgaggaataccgaatggctgagaagcacctgaaaaaatgttcagcatccttaatcatcagagaaatgcaaatcaaaacaaccctgagattctgtctcacaccagtcagaatggctaagatcaaaaattcaggtgacagcagatgctggcgaggatgtggaggaagaggaacactcctccattgtttgtgggattgcaagcttgtacaaccactctggaagtcagtctgtgggttcctcagaaaattggacatagtactaccggaggatcctgcaatacctcttctgggcatatatccagaagatgttccaactggcaagaaggacacatgctccactatgttcatagcagccttatttataatagccagaagctggaaagaacccagatgcccctcagcagaggaatggatacaaaaaatgtggtacatttacacaatggagtactactcagctattaaaaggaatgaatttatgaaattcctggcaaatggttggacctagagggcatcatcctgagtgaggtaacacaatcacaaaagaactcaaatgatacatactcaccgataagtggatattagcccaaaagcttagtatacccgagatataagatacaatttgcaaaacacatgaaactgaagaagaacgaagaccaaagtgtggacactttgtcccttcttagaattgggaacaatcacccatggaaggagttacagagacaaagtttgaagctgagacaaaaggatggaccatctagagactgccatatccagggatccatcccataatcagcctccaaatgatgacaccattgcatacactagcaagagtttactgaaaggaccctgatatagctgtctcttgtgagactagaccggggcctagcaaacacataagtggatgctcacagtcagctattggatggatcacagggcccccaatggaagagctagagaaagtatccaaggatctaaagagatctgcaggtgcaacaacattatgaactaaccagtatcccggagctcttgactctagctgcatatgtatcaaaagatggcctaatcggccatcactggaatgagaggcccattggacatgcaaactttatatgccccagtacaggggaacaccagggccaaaaagtgggaatgggtgggtaggggagtgtgggggagggtatgggggacttttgggatagcattggaaatgtaattgaagaaaatatgtaataaaaaaattaaaaaaaaaaaagaacttcaagtctctggagaaagaaattgcagatctcagaagatggaaagatctcccatgctcatgggttagcagtattaatatagtaagaatggctgtcttgctgaaagcaatctacagatttaatgcaatcaccatcaaaattccaactcaattcttcactgagatagaaagggcaatttgcaaattcatctggaataataaaaaacctaggatagcaaaaaatattctcaacaataaaagaacctctggtggaatcaccatgcctgacgttaagaaggacacatgctccactatgttcatagcagccttatttataatagccagaagctggaaataacccagatgtccctcaacagaagaaaggacacagaaaatgtggtacatttacacaatggagtactactcaactattaaaaacaatgaatttatgaaattcttggacaaatggatgtatctggaagatatcatccttagtgaggtaacccaatcacaaaaaagtcattagatatgcactcactgattactggatattagcccagaaacatagaacactcaagatacaatttgcaaaatacaagaaaatcaaaaagagggatgaccactgggtggatacttcattcctccttagtatagggaacaaaatacccatgaaaggagttacagagacaaagtttggagctaagacgaaaggatgggctatccagagactaccccacatggggatccatcccataatcagccaccaaacccagatactattgcatatgccagcaagattttgatggagggaccctggtatagctgtctcatatgaggctatgccagtgcctggcaaatacagaagtggatgctcatagtcatctattggatggaatacagggcgcccaatggagaaactagagaaagcacccaaggagctgaaggggtgggcaaccctataggtggaacaacaatatcaactaaccagtacccccagagctcgtttctctagctgcatatacagcagaagttgaccatcactgggaagagaagcctcttggtcttgcaaactttatatgccccagtacaggggaatgccacagccaagaagtgggagtgggtgggtaggggagcagggcagggggatggtatagggaactttcgggatagcatttgaaatttgaaatgtatataaagaaagtatctaataaaaaggaaatctttaagacctaagagctaagtgtggcagcacacgcttatgatatctgtgctgggagacagagataggtggttactagggctcataggtcagccaaccttgcatacttggcaagatcctggctcttatgggacattgtaccaaaaaataagtggacatgtgtgcaaatacacataaacctaataagggttggggagacagttcagtgattaaaatgcttactatggtagtgtgaaggccagagtttatctccctagaacccacataaaatgtcaggtggatatggaatcctacttataattccagcctgggaatacagagacaagagatcccaagaacaagttggctagggagattcatcatattggtgagctctgggtttgattgagagaccctgcctcagtgaataagatagaagagtaaccaaggatgagccaagacatcaacttaggcttccacatgcacacatatatacaagcaaatatacatacttatgcatgccacacatgcacatgaagattaaaaacagtagaataatagcattttactgagtcaaaatagaaaaattattaatatagtattactcttggtttcctgttaaactaatgatcaaatccaaacacgtgcatgttacacatgggaatgcaaagactaaaatccccatattggggagtcctcagaccttttgctctgtgaatctcccccatgccttcccctgaatgaatttaaatgttaacccagaaatgggttgcagactgagcagttgatggtcagaataatcttttagatccaaaatgtggttaaatgtgttatgtgaaccaagtttttgcctgaagtcctaggtcaatggtaaagggagagctgtgaacctttctattaaaaaataaaaatgtgtgaaagtcccatggtgactagtgtcaggcctcacccaccacatcctgtgcatctccctcacccactggactcaatccacacacatagttcagcactggtctggccactggtatg

SEQ ID NO: 4 - DNA, 4048 bp, synthetic construct
  source 1..4048: mol_type = other DNA
atatacccctcttactctctttcaaattcatggcctctagtttgtgacaagaaaaatgtcatcttaagcaaaaacactctctctttctctctctctctcttgctcttgctctctctggtctatggatctagatataaagtctgtcttgtttctgccatgatgctcatagggtagttctctaaagctgtgaacaagcccccaattaaatactttcttttataagagttactttggtcatagactttcgtctcgtcactagaacagtaactaaggcagactgtcttagttagggttttattgttatgaaaagacataatgaccaatgaaactcttataaaggaaaacatttaattggggctgacttacagttcaggggtttagtcctttatcatcacggcaggaagcatggtggcatgctagaagacaaggtattagagaagaagctaagaatcctataccttgatccacaagcaacaggagactgtttgccacaatgggcataggtggagcataggagacctcaaagcctgcccccacagtgacacacttcctccaacaaggccacacctactccaacaaggtcacacttcagaatagtgccactccctatgggccaagcattcaaatacattagtctatgggggctattaatattcaaaccaccactcagaatacttctcgccctctttcattcatggatctggacctcagtcctccatgatattgcccaacacaactatgttatgtgctgactccccaaagccctgcagattgtcttctttccaccattgcttcttctttaactgctattgtaataatcactaataagctcccaataacacacaatgagatctgcacctcttgttggttctagctaacactttgcccacttcttgaatcacttctctagtttctcgctgcttatctccaaatggttatgatactgttatgttttctgtcaggaaccatctaggggcagcctaccttttttgcatggcctcagatgagaggctctgagaggcaaagtcccaaatacatttcacctcaggatttcttcttgcagctgatatgcttttttcctatcacgatcacatagcctgacacttcctgggcatcagcacaccctattgggcagatttcctgaagatcaacatgaagatgaactttcatgaattaatgctatttagaagaattgattattttatagagttccagatttgattcaaataatctcagaaagaacatttcaagagctgttttttgttgtttgctagaaagatataataaagttactatcaaggatagaatgcacccactccccataataataagtaaggctgcataataagaaatacaatgagctttcatattgcactaaaaggagatataggcttgggaaaaatttgtgttcaaggaaaagcatccaggtacaaggatgaagccccaagtgtgcactctgagaaaacaaggccatgtaaaactgttgctttgaacttagaatgagcatatagaatgaatcactgctttgaacttagcatcaacatctttttgtaactcccattttgtctaacattttatctctgcggtaactttctaaaatcctttctctataagaaaataaataaaaatgatggaatggcttggctctatgccagatacatctgtccatccatctgtgcatctgtctgtccatccatccatccattcacctgtctgtccatccatccatatgtagtgatgtagccattttctgcttcttctggtttgggtgtgtgttctttctgtgtgaatgactctttctttcctttctggttcacaaagagttaaccagcctgacaagtgaggggctcttgggctactagaaagcatgctagcaataaatcctttacttaatcccttgctgttaagcaacaggtgttaattcttcagaagccattggcttctggccccagaataagaaagaaccaaagagaatagatatcaatactaagtaatctttatatttgcaaaaccactctccaccagccatcatctgccttccggagaggactcagccagggtaagctccagccagttttcaagcttcttcaactccacttctatttaatatacttttattagttttattttttatgtgaagagtgtgtatgtgtgcgcacacatgctcacatgcctgtgaaggcagacgcctgtgaagaacagaagatggcctcagatctactatctccttcctggtccactaagctgtgaacaccatctaccataacagaaacacctgcttgtatgccttcctgtccgtgatggactgaatactttcttttgtgttctttaaaagataaggagagacagaatatgtttggtgggtgtgtagtcaggtacaggggaaaagggtgcctccgggggtgcaatctgaggcatcccttccccctaagggaccagcctcatgaaggtatagtatagaatagagtttattcagggcatggagagggatattgagagggtaatagagccaaaagggggggggggggggaagaggagtacaggccagccatgagcacgtggagagggagggggaggggaatggggaggggaggacaggcaggaggacagagtaggagtaagaaggcaagaggggggggggcgggaagcagccccttttatagtgagtcaagctgttgccaagtaactgtggggcagagcttagacaaaatgttaacagcaataagaaaagcagctgatagagtaagctaattgagtctttcagtccaatgccagatctctttagcattttgtgatactatgcaatattatagatgcaaacctgggttcatgccagccaagtgtattgcttcaactttcctaagactgatgtccaaatctatttctctgaaccctcaaacttttacccttagaacttcagataatattgtcagcttattccatcaaacttgggttcataatttcttcatttttttatttgtgtgtgtgcacacatacacgtgtgcaactgctcatggagttgtgaatcagatttgctggagctggacacacacagacagatacacaggcactcatagacacatacatacacagacacacacacacagacatacaacacacgcttagacacacagatatacacatagacacacatagcaccacaagaggttactctctttctctaccgccatgaccatagcacctattgaaatgcaggtctcatttcccaaacaatagaatagcaaggattggagactggtctgccctgtttccagctacagctgcagctcactgctaatacttgagagaacttttttgtgcgatggaaatgttctaagaatgagctactgtggtgctagcatccacaatctgcacttaacaggagggagtttcattgcatataaactacattcaataaaactttttataagagtacaactatcacacacacataccatatcaaggcttaggaaccaacatggaagagagcagagaaattgtaaaacactgctctcatgaacacaatagaactggatcactgtactcaggaaaccagaacagctgtggttgcctgtccatgacgaagtactgcagccattccatactgcaaggtgggcagagagatttccactcgtaaacaaggaatttctgaggaaattctggtgaacctcttcagattccgtatcaccaatgttgtgagtctcagctaaggtcacccttattgattcttgggtttttcccctatcccaggtcctctacatcctagagatgccaccctcctcccaacccctgccacttgcagatttccattcattctcatggtcatctggccatctatcccacacacccagcagttgactcagaaagatgcagacacccacagctgccacggcactccggtcaagtcagcttgtcaggcagtgatgcctaaaagtcactcatgatgtaaagagtttcaagaaagctctcctcctggagtctggcattctctctaacccatacattaatttttcattcccgagttagtctagtgtatggatccacatgc

SEQ ID NO: 5 - DNA, 1420 bp, synthetic construct
  source 1..1420: mol_type = other DNA
gggaccctggtatagctgtctcatatgaggctatgccagtgcctggcaaatacagaagtggatgctcatagtcatctattggatggaatacagggcgcccaatggagaaactagagaaagcacccaaggagctgaaggggtgggcaaccctataggtggaacaacaatatcaactaaccagtacccccagagctcgtttctctagctgcatatacagcagaagttgaccatcactgggaagagaagcctcttggtcttgcaaactttatatgccccagtacaggggaatgccacagccaagaagtgggagtgggtgggtaggggagcagggcagggggatggtatagggaactttcgggatagcatttgaaatttgaaatgtatataaagaaagtatctaataaaaaggaaatctttaagacctaagagctaagtgtggcagcacacgcttatgatatctgtgctgggagacagagataggtggttactagggctcataggtcagccaaccttgcatacttggcaagatcctggctcttatgggacattgtaccaaaaaataagtggacatgtgtgcaaatacacataaacctaataagggttggggagacagttcagtgattaaaatgcttactatggtagtgtgaaggccagagtttatctccctagaacccacataaaatgtcaggtggatatggaatcctacttataattccagcctgggaatacagagacaagagatcccaagaacaagttggctagggagattcatcatattggtgagctctgggtttgattgagagaccctgcctcagtgaataagatagaagagtaaccaaggatgagccaagacatcaacttaggcttccacatgcacacatatatacaagcaaatatacatacttatgcatgccacacatgcacatgaagattaaaaacagtagaataatagcattttactgagtcaaaatagaaaaattattaatatagtattactcttggtttcctgttaaactaatgatcaaatccaaacacgtgcatgttacacatgggaatgcaaagactaaaatccccatattggggagtcctcagaccttttgctctgtgaatctcccccatgccttcccctgaatgaatttaaatgttaacccagaaatgggttgcagactgagcagttgatggtcagaataatcttttagatccaaaatgtggttaaatgtgttatgtgaaccaagtttttgcctgaagtcctaggtcaatggtaaagggagagctgtgaacctttctattaaaaaataaaaatgtgtgaaagtcccatggtgactagtgtcaggcctcacccaccacatcctgtgcatctccctcacccactggactcaatccacacacatagttcagcactggtctggccactggtatg

SEQ ID NO: 6 - DNA, 1515 bp, synthetic construct
  source 1..1515: mol_type = other DNA
cagaatctcattcgaatcggactggcatgcataatcctgataactctagtatggcttttgactgaagactggctcagcaagaggaaagatcatgaagaggccaacagattaacaaattgggaatgcaggagaagatggagaatgcagcattactttgaagaggaacaaagaaatgcaatatctatgatggaactgaaggcaactcctggggccttgtgagctctatgctgtaccctggataaagaaagagatcttttcagaaatgaaggggtaggagtgttaccaagtttggcaaagcattctaaaggacactgtgagaggaagcctctaacagacctctctgtctctgctaagctttaagtactttaacgcttatttactgcaagttatacttttgatcactctggtctttcctagaaataacttcgttccaatgtgatggccatgggaatatttccttcctactttaggtcttatcgtcatttctttttctaattttatttattttatgttgccgggaattgaactcaagttgtctggaggagcaacaagtgctcttaactacaaaccatatctccagctccatcctcaatattttatattgtgtgtgcatgtctgtaggatgcatgtgtgtgcacacatatgtacatatattctatgccattaaaatgaaggtcagagaacacctctgtgaagtcagttttctctttctttttttcttttcttttttttttttttgttgtttttttttttgtttttttttttgtttttttgaggcagggtttctctgtgtagccctggctgtcctggaactcactctgtagaccaggctggcctcgaactcagaaatccgactgcctctgcctcccgagtgctgggattaaaggcgtgcaccaccacgcccggctgtgaagtcagttttctatttccactttctaacaattcttttatttttgagaatataatttaaacatctgtgttttttttttaaagttatatctcctccacccttcactttcttctcttcaaacactcccatatacccctcttactctctttcaaattcatggcctctagtttgtgacaagaaaaatgtcatcttaagcaaaaacactctctctttctctctctctctcttgctcttgctctctctggtctatggatctagatataaagtctgtcttgtttctgccatgatgctcatagggtagttctctaaagctgtgaacaagcccccaattaaatactttcttttataagagttactttggtcatagactttcgtctcgtcactagaacagtaactaaggcagactgtcttagttagggttttattgttatgaaaagacataatgaccaatgaaactcttataaaggaaaacatttaattggggctgacttacagttcaggggtttagtcctttatcatcacggcaggaagcatggtggcatgctagaagacaaggtattagagaagaagctaagaatcctataccttgatccacaagcaacagg

SEQ ID NO: 7 - DNA, 6525 bp, synthetic construct
  source 1..6525: mol_type = other DNA
tcttccacactccctgccctgctctgcgtcggtgagttctggcgtggaaggggaatgggatcacggtgtgcctgggaggcaacaggtctcattactcccgtcttccagggctgtgtctgagtcagaggatcagcgcccagcagcgtgagtccttccttcaaagcccagggtcactcttccggattcaggccaagctccttccacccaagcacggctggggtgaggggacagggtgctggcttcccaggagagcttggggccagcagctgggtggagcctaaggttggggggagggggctccgctggaactccagcctctgattcccttccagagactctcccaaaaccgttcatctgggccgagccccatttcatggttccaaaggaaaagcaagtgaccatctgttgccagggaaattatggggctgttgaataccagctgcactttgaaggaagcctttttgccgtggacagaccaaaaccccctgagcggattaacaaagtcaaattctacatcccggacatgaactcccgcatggcagggcaatacagctgcatctatcgggttggggagctctggtcagagcccagcaacttgctggatctggtggtaacaggtaactgtccggttctctaactggagagtgatctcagtctgcatccgggatgcagcatcatctatgaactcttccaagccccactcagacactgcttgtctcggtaggaggctggaaggaggggtgatccccatcacaatccttgcctacaaggggttgtctgcagaccgtgtctctacgtcctaggagcagatgtgtcctcagtcagtttctccatgacacagattctgagatagatatttgtatgcaggggtatgactgaggaatgtcctcaaaaacaatgcctgtgggccaggcgcagtggcttacactttgcttccctcacccatcacaggtggtgggttttttttttttttatctgttttgagacggagtttcgctcttgtcacccaggctggagtgcagtggtgcaatctccagtcactgcaacctccacctcctgggttcaagtgattctccagcctcagcttcccaagtagctgggatcacaggcacccaccactacgccacattttgtatttttagtagagatggggtttcaccatgttggccagggtggtgtcgaactcctgacctcagatgatccgcccgcctcaccctcccaaagtgctgggattacaggtgtgagccatcacacccagccaggtggtggttttctaaaaaaaaaaaaaaattagcttttttttttttttaacaatatggttgtttattattattatcaagtattatacatagttacatatacatacataattgtatgtgctatacaattaggtttgtttataccagcaacaccaaaaacacatgagcaatactttgtgctaggaaggctatgatgtcatcaggcaataggaatttttcagtttcattataatcttatgggaccaccatcatatatgtggtacattgttggccaaaatgtcattatgcagctcacaacagtatttcatgtccattcaaatatcttcttttgtgaaatgtctatttaaatcttttgcctatttttaaattgggttgcttatattttgattgattaggaaaagttatttctatattctgtgtcatatacttgtgttgaaatatatatattttttgtctgtgccttttcatttgctcagggtctttggaccttgtttggaggttctggcaggggaacacagctactcatttattcttttttttttaatttttttagtatttattgatcattcttgggtgtttctcggagagggggatttggcagggtcataggacaatagtggagagaaggtcagcagataaacatgtgaacaaaggtctctggctttcctaggcagaggtccctgcggccttccgcagtgtttgtgtccctgggtacttgagattagggagtggtgatgactcttaaggagcatgctgccttcaagcatctgtttaacaaagcacatcttgcaccgcccttaatccatttaaccctgagtggacacagcacatgtttcagagagcacggggttgggggtaaggtcatagattaacagcatcccaaagcagaagaatttgtcttagtacagaacaaaatggagtctcctatgtctacttctttctacacagacacagtaacaatctgatctctctttcttttccccacatttcccctttttctattcgacaaaaccgccatcgtcatcatggcccgttctcaatgagctgttgggtacacctcccagacggggtggcggccgggcagaggggctcctcacttcccagacggggcggccgggcagaggcgcccccccacctcccagacggggcagtggccgggcgggggctgccccccaacctcccggacggggcggctggccggggcttttttttttttttttgagacagtctcgctgcagtgcagtggtacaatctcagctcactgcaacctctgcctcagcctcaattctcctgcctcagcctcccaagtagttgagattacaggcatgtgccaccacacccggctaatttttgcatttttagtagagacggggtttcaccatgttgaccaggctggtctcaaactcctgacccaggaggtcgaggcttcagtaagcaaagatagtgccacggcgctccagcctgggaaacagagcaagaccctgtatcatttttaaaaatggttttagacggtaaatcttctattgtgtgtatttgaccaaaataataattaaaaaaaaaaaaaaaagctggctgccaggcatggtggcaggcccctgtagtcccagctacttgggagggtgaggcaggagaaacgcttgaacccgggaggcggaggttgcagtgagccaagatcgtgtcactgcactccagcctgggcgacagagagagactccatctctaaagaaagaaaaaaaaaaatagctggctgctcatcactgagtttctggtgtggtggccccaccttctctcatagaaatgtatgacacacccaccctctcggttcatcctggacccgaagtgatctcgggagagaaggtgaccttctactgccgtctagacactgcaacaagcatgttcttactgctcaaggagggaagatccagccacgtacagcgcggatacgggaaggtccaggcggagttccccctgggccctgtgaccacagcccacagagggacataccgatgttttggctcctataacaaccatgcctggtctttccccagtgagccagtgaagctcctggtcacaggtgaggaaatgctcaattccccacacccttcgccgccatgtgctacctggagccctgagggatccccagagagtgatggggagggtgtccaagggacgtccacttcctgggtgcctggttggtcatgtgaggaagaacaccagaagcaggaaggaggagggaacagagaaaggaatggtaaggcgggtggatcacaaggtcaggagttcgagaccagcctggccaagacggtgaaaccccgtctctactaaaaatacagaaattagccagacgcagtggcggacacctgtagtcccagctactcaggaggctgaggcaggagaatcgcttgaacccgggaggcgggggttgtagtgaaccgagatcataccaccgcactgcaacctgggcgacagagcaagactccatctcaaaaaaaaaaaaaaaaaaaaaagaatggcaagaccggaggaaaccaaaaacccttacttttttttctttatctccttttccaggcgacattgagaacaccagccttgcacctgaagaccccacctttcctggtgagtaactggtccttctaagctcagacgagcgatcagagcctcccagtgacactaaaaacgtggcattcattcaaaatattcatcgaggccaggcgtggtggctcacgcctgtaatcccagcactttgggaggccgagatggtgcatcatttgaggtcaggagtttgagaccagcctggccaacatggcgaaaccctgtctctactaaaaatacaaaacttaggctgggcatcatggctcacacctgtaatcccaacacttcgggaggccaaggtggttggatcacaaggtcaggaattcgagaccagcctgaccaacatggtgaaaccccatctctactaaaaatacaaaaattagccgggcctggtggtgctcgcctgtaatcccagctactcaggaggctgaggcaggagaattgttgaacctgggatgcagaggttgcagtgagctgagatcgcgccactgcattccactccactgcacgacacagcgagactccatctcacagaaaaacaaaaacaaaactattatatatatatattcatcaagtgcatagtatacacagtgaactacactgtaacagtcagccaggcagatatcttgactgtgcagcacttagattctagcaggaggagacacaccatcggtcaacgtcaggatagcacacaggagggaatgatgctatggaaggaaaagacaaagtagaacagacttacagtgattgaaatggcagctagcaatattaaataggtttgtccagatggacctcacagagaaagaaggcatctgagcaaatgcgttcagacttgagttaatcatgtggctgtcaggagaaaggaggctctggagagaatgaaatggcatctgcctgtgccctggggcaggaagataactggggtaatacaataataactatgaggccaggagggttgaaaatgatgtttggaagatgacggtgggatgggcctggggcgcacggctaggattacaggagtgaggcccggcgcggtggctcacgcctgtaatcccagcactttgggaaaccgaggcaggtggatcatgaggtcaggagatcaagaccatcctggctaacacggtgaaaccctgtctctactaaaaaaaaatacaaaaattatccgggcgtggtggcgggcgcctgtagtcccagctacgcaagaggctgaggcaggagaatggcgtgaacccgggagacggagcttgcagtgagctgagatcgcgccactgcactccagcctgggcgacagagtgagactccgtctcaaaaaaaaagaaaaagaaaaagaaaaagaaaaaaaaatagtgagactttgaatttcactatgtgtgaggagaaagaggtaatgatgacttaatgaggaaaatgaggcttaaatagaagacgggctgggccgggtggctcctgcatgtaatcccagcactttggaaggcaggggcggctggatcacttgaggtcaggagttcaagaccagcctggccaacacagtgaaaccccatctctactaaaaatacaaacatgagttgggtgtggtggcgcacgccagtaattacagctactcggggctgaagcaagaggattgcttgaactcgggaggcggaggttgcagtgagctgagatcacaccactgtactccagcctcagaggcctgccatcccagccctttgggaggccgaagcaggcaggtcatctgaggttgggagttcaagaccagcctggccaacatggcaaaaccccgtttctactaaaaatatgaaaaaaattacctgggtatgtggtgtgtgcctgtagtcccagctactccagaggctggaacacagtgagactctatctcaaaaaaaaaaaaaatagaagacatgactggtgcaaagacacatgctcacaagtgctagaatggaattcctcgtcaggttcgtccatctgtggacccttccactttacctgctggatgaagctcctgggacccgcagggtgaggtgggaccttgtaaagctgcagaacgtcatggggtagacccaagggaaggagtgctggggtggaggaggtcaaaaccatcctcttttcttcacttcccttatcatcagcagacacttggggcacctaccttttaaccacagagacgggactccagaaaggtaagtagacagctggggccataggctctgaaggaaggggctgggcatagagtagacctaggaagggaatctaaatgggaacaagagggtgtccttggccaggcgcagtagctcacacctgtaatctcagccctttgggaggccgaggcgggcagatcatctgaggtcgggagttcaagaccagtctggccaacatggcgaaatcccatctctactaaaaatacaaaaaaattagccaggcgtggtggcgtgtgcctgtagtcccagctacttgggaggctgagacaggagaatagcttgaacccaggaagtggaggttgcagtgagccgagatcgtgccattgcactccagcctgggcgacaagactgaggctctgtctcaaaaaaaaaaaaaaaaaaaaaaaaaaaaaaaaaaaaaaagagggtgtccttacatccctgtcagcgatcaccctgttctcctgcctacagaccatgccctctgggatcacactgcc

SEQ ID NO: 8 - RNA, 1463 nt, synthetic construct
  source 1..1463: mol_type = other RNA
gttcagcactggtctggccactggtatgtcttccacactccctgccctgctctgcgtcgggctgtgtctgagtcagaggatcagcgcccagcagcagactctcccaaaaccgttcatctgggccgagccccatttcatggttccaaaggaaaagcaagtgaccatctgttgccagggaaattatggggctgttgaataccagctgcactttgaaggaagcctttttgccgtggacagaccaaaaccccctgagcggattaacaaagtcaaattctacatcccggacatgaactcccgcatggcagggcaatacagctgcatctatcgggttggggagctctggtcagagcccagcaacttgctggatctggtggtaacagaaatgtatgacacacccaccctctcggttcatcctggacccgaagtgatctcgggagagaaggtgaccttctactgccgtctagacactgcaacaagcatgttcttactgctcaaggagggaagatccagccacgtacagcgcggatacgggaaggtccaggcggagttccccctgggccctgtgaccacagcccacagagggacataccgatgttttggctcctataacaaccatgcctggtctttccccagtgagccagtgaagctcctggtcacaggcgacattgagaacaccagccttgcacctgaagaccccacctttcctgcagacacttggggcacctaccttttaaccacagagacgggactccagaaagaccatgccctctgggatcacactgcccagaatctcattcgaatcggactggcatgcataatcctgataactctagtatggcttttgactgaagactggctcagcaagaggaaagatcatgaagaggccaacagattaacaaattgggaatgcaggagaagatggagaatgcagcattactttgaagaggaacaaagaaatgcaatatctatgatggaactgaaggcaactcctggggccttgtgagctctatgctgtaccctggataaagaaagagatcttttcagaaatgaaggggtaggagtgttaccaagtttggcaaagcattctaaaggacactgtgagaggaagcctctaacagacctctctgtctctgctaagctttaagtactttaacgcttatttactgcaagttatacttttgatcactctggtctttcctagaaataacttcgttccaatgtgatggccatgggaatatttccttcctactttaggtcttatcgtcatttctttttctaattttatttattttatgttgccgggaattgaactcaagttgtctggaggagcaacaagtgctcttaactacaaaccatatctccagctccatcctcaatattttatattgtgtgtgcatgtctgtaggatgcatgtgtgtgcacacatatgtacatatattctatgccattaaaatgaaggtcag

SEQ ID NO: 9 - protein, 325 aa, synthetic construct
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLIRIGLACIILITLVWLLTEDWLSKRKDHEEANRLTNWECRRRWRMQHYFEEEQRNAISMMELKATPGAL

SEQ ID NO: 10 - DNA, 97 bp, synthetic construct
  source 1..97: mol_type = other DNA
ctcctccacccttcactttcttctcttcaaacactcccggtaccctcgaggtcgacggtatcgataagcttgatatcgaattccgaagttcctattc

SEQ ID NO: 11 - DNA, 81 bp, synthetic construct
  source 1..81: mol_type = other DNA
tataggaacttcatcagtcaggtacataatggtggatccatatacccctcttactctctttcaaattcatggcctctagtt

SEQ ID NO: 12 - DNA, 26 bp, synthetic construct
  source 1..26: mol_type = other DNA
atcaccatgcctgacgttaagaagga

SEQ ID NO: 13 - DNA, 25 bp, synthetic construct
  source 1..25: mol_type = other DNA
ttcttcctcacatgaccaaccaggc

SEQ ID NO: 14 - DNA, 23 bp, synthetic construct
  source 1..23: mol_type = other DNA
ttgaatcaagagcagattggggg

SEQ ID NO: 15 - DNA, 23 bp, synthetic construct
  source 1..23: mol_type = other DNA
gaatctcattcgaattggtctgg

SEQ ID NO: 16 - DNA, 20 bp, synthetic construct
  source 1..20: mol_type = other DNA
ttgaatcaagagcagattgg

SEQ ID NO: 17 - DNA, 24 bp, synthetic construct
  source 1..24: mol_type = other DNA
taggttgaatcaagagcagattgg

SEQ ID NO: 18 - DNA, 20 bp, synthetic construct
  source 1..20: mol_type = other DNA
ccaatctgctcttgattcaa

SEQ ID NO: 19 - DNA, 24 bp, synthetic construct
  source 1..24: mol_type = other DNA
aaacccaatctgctcttgattcaa

SEQ ID NO: 20 - DNA, 20 bp, synthetic construct
  source 1..20: mol_type = other DNA
gaatctcattcgaattggtc

SEQ ID NO: 21 - DNA, 24 bp, synthetic construct
  source 1..24: mol_type = other DNA
tagggaatctcattcgaattggtc

SEQ ID NO: 22 - DNA, 20 bp, synthetic construct
  source 1..20: mol_type = other DNA
gaccaattcgaatgagattc

SEQ ID NO: 23 - DNA, 24 bp, synthetic construct
  source 1..24: mol_type = other DNA
aaacgaccaattcgaatgagattc

SEQ ID NO: 24 - DNA, 132 bp, synthetic construct
  source 1..132: mol_type = other DNA
gaattctaatacgactcactatagggggtcttcgagaagacctgttttagagctagaaatagcaagttaaaataaggctagtccgttatcaacttgaaaaagtggcaccgagtcggtgcttttaaaggatcc

SEQ ID NO: 25 - DNA, 25 bp, synthetic construct
  source 1..25: mol_type = other DNA
gcagtgtttgtgtccctgggtactt

SEQ ID NO: 26 - DNA, 25 bp, synthetic construct
  source 1..25: mol_type = other DNA
tacccaacagctcattgagaacggg

SEQ ID NO: 27 - DNA, 25 bp, synthetic construct
  source 1..25: mol_type = other DNA
tctaccgccatgaccatagcaccta

SEQ ID NO: 28 - DNA, 25 bp, synthetic construct
  source 1..25: mol_type = other DNA
tctgctctcttccatgttggttcct

SEQ ID NO: 29 - protein, 325 aa, Rattus norvegicus
MLPTLTALLCLGLCLSQRINTEKQTLPKPIIWAKPSIMVTKGNSVNIWCQGAQSASEYQLYFEGSFFALERPKSSRSMNKVKFFISQMTSHTAGIYTCFYQSGELWSESSNPLKLVVTGLYDTPTLWVHPGPEVTLGENVTFSCHLKTATSKFFLLKERESNHIQHKYGNIQAEFPMGPVTRAHRGTYRCFGSYNDYAWSFPSEPVTLLITGEVENTSLAPTDPVSSLDYWEFDLSTKESGLQKDSAFWDHTAQNLIRIGLACIIVMALVWLLAEDWLSRRKDHEKLNRLTSWECRGRRRMHRYHEEEQRDAISMRELKATPGDM